SEQUENCE LISTING (150 sequences)

SEQ ID NO: 1 - RNA, 21 nt, Homo sapiens
uggaauguaaagaaguaugua

SEQ ID NO: 2 - RNA, 23 nt, Homo sapiens
ucuuugguuaucuagcuguauga

SEQ ID NO: 3 - RNA, 23 nt, Homo sapiens
uacccuguagauccgaauuugug

SEQ ID NO: 4 - RNA, 22 nt, Homo sapiens
uacccuguagaaccgaauuugu

SEQ ID NO: 5 - RNA, 22 nt, Homo sapiens
uagcagcacauaaugguuugug

SEQ ID NO: 6 - RNA, 22 nt, Homo sapiens
uagcagcacaucaugguuuaca

SEQ ID NO: 7 - RNA, 22 nt, Homo sapiens
uagcagcacguaaauauuggcg

SEQ ID NO: 8 - RNA, 23 nt, Homo sapiens
caaagugcuuacagugcagguag

SEQ ID NO: 9 - RNA, 23 nt, Homo sapiens
uaaagugcuuauagugcagguag

SEQ ID NO: 10 - RNA, 22 nt, Homo sapiens
uagcuuaucagacugauguuga

SEQ ID NO: 11 - RNA, 22 nt, Homo sapiens
aagcugccaguugaagaacugu

SEQ ID NO: 12 - RNA, 21 nt, Homo sapiens
aucacauugccagggauuucc

SEQ ID NO: 13 - RNA, 21 nt, Homo sapiens
aucacauugccagggauuacc

SEQ ID NO: 14 - RNA, 23 nt, Homo sapiens
gugccuacugagcugauaucagu

SEQ ID NO: 15 - RNA, 22 nt, Homo sapiens
uucaaguaauccaggauaggcu

SEQ ID NO: 16 - RNA, 21 nt, Homo sapiens
uucaaguaauucaggauaggu

SEQ ID NO: 17 - RNA, 21 nt, Homo sapiens
uucacaguggcuaaguuccgc

SEQ ID NO: 18 - RNA, 21 nt, Homo sapiens
uucacaguggcuaaguucugc

SEQ ID NO: 19 - RNA, 21 nt, Homo sapiens
uagcaccaucugaaaucgguu

SEQ ID NO: 20 - RNA, 22 nt, Homo sapiens
uagcaccauuugaaaucgguua

SEQ ID NO: 21 - RNA, 22 nt, Homo sapiens
uguaaacauccucgacuggaag

SEQ ID NO: 22 - RNA, 22 nt, Homo sapiens
cuuucagucggauguuugcagc

SEQ ID NO: 23 - RNA, 22 nt, Homo sapiens
uguaaacauccuacacucagcu

SEQ ID NO: 24 - RNA, 23 nt, Homo sapiens
uguaaacauccuacacucucagc

SEQ ID NO: 25 - RNA, 22 nt, Homo sapiens
uguaaacauccccgacuggaag

SEQ ID NO: 26 - RNA, 22 nt, Homo sapiens
cuuucagucggauguuuacagc

SEQ ID NO: 27 - RNA, 20 nt, Homo sapiens
uguaaacauccuugacugga

SEQ ID NO: 28 - RNA, 21 nt, Homo sapiens
ggcaagaugcuggcauagcug

SEQ ID NO: 29 - RNA, 22 nt, Homo sapiens
uauugcacuugucccggccugu

SEQ ID NO: 30 - RNA, 23 nt, Homo sapiens
caaagugcuguucgugcagguag

SEQ ID NO: 31 - RNA, 22 nt, Homo sapiens
uucaacggguauuuauugagca

SEQ ID NO: 32 - RNA, 22 nt, Homo sapiens
aacccguagauccgaucuugug

SEQ ID NO: 33 - RNA, 22 nt, Homo sapiens
cacccguagaaccgaccuugcg

SEQ ID NO: 34 - RNA, 22 nt, Homo sapiens
aacccguagauccgaacuugug

SEQ ID NO: 35 - RNA, 22 nt, Homo sapiens
uacaguacugugauaacugaag

SEQ ID NO: 36 - RNA, 24 nt, Homo sapiens
aaaagugcuuacagugcagguagc

SEQ ID NO: 37 - RNA, 21 nt, Homo sapiens
uaaagugcugacagugcagau

SEQ ID NO: 38 - RNA, 23 nt, Homo sapiens
agcagcauuguacagggcuauca

SEQ ID NO: 39 - RNA, 23 nt, Homo sapiens
uggagugugacaaugguguuugu

SEQ ID NO: 40 - RNA, 24 nt, Homo sapiens
ucccugagacccuuuaaccuguga

SEQ ID NO: 41 - RNA, 22 nt, Homo sapiens
acaggugagguucuugggagcc

SEQ ID NO: 42 - RNA, 22 nt, Homo sapiens
ucccugagacccuaacuuguga

SEQ ID NO: 43 - RNA, 22 nt, Homo sapiens
ucguaccgugaguaauaaugcg

SEQ ID NO: 44 - RNA, 22 nt, Homo sapiens
ucggauccgucugagcuuggcu

SEQ ID NO: 45 - RNA, 22 nt, Homo sapiens
cugaagcucagagggcucugau

SEQ ID NO: 46 - RNA, 22 nt, Homo sapiens
cagugcaauguuaaaagggcau

SEQ ID NO: 47 - RNA, 22 nt, Homo sapiens
cagugcaaugaugaaagggcau

SEQ ID NO: 48 - RNA, 22 nt, Homo sapiens
uaacagucuacagccauggucg

SEQ ID NO: 49 - RNA, 22 nt, Homo sapiens
uugguccccuucaaccagcugu

SEQ ID NO: 50 - RNA, 21 nt, Homo sapiens
uugguccccuucaaccagcua

SEQ ID NO: 51 - RNA, 23 nt, Homo sapiens
uauggcuuuucauuccuauguga

SEQ ID NO: 52 - RNA, 22 nt, Homo sapiens
cagugguuuuacccuaugguag

SEQ ID NO: 53 - RNA, 21 nt, Homo sapiens
uaccacaggguagaaccacgg

SEQ ID NO: 54 - RNA, 22 nt, Homo sapiens
uaacacugucugguaaagaugg

SEQ ID NO: 55 - RNA, 23 nt, Homo sapiens
uguaguguuuccuacuuuaugga

SEQ ID NO: 56 - RNA, 21 nt, Homo sapiens
cauaaaguagaaagcacuacu

SEQ ID NO: 57 - RNA, 22 nt, Homo sapiens
ugagaugaagcacuguagcuca

SEQ ID NO: 58 - RNA, 23 nt, Homo sapiens
guccaguuuucccaggaaucccu

SEQ ID NO: 59 - RNA, 22 nt, Homo sapiens
ugagaacugaauuccauggguu

SEQ ID NO: 60 - RNA, 22 nt, Homo sapiens
ugagaacugaauuccauaggcu

SEQ ID NO: 61 - RNA, 22 nt, Homo sapiens
ucagugcacuacagaacuuugu

SEQ ID NO: 62 - RNA, 22 nt, Homo sapiens
ucagugcaucacagaacuuugu

SEQ ID NO: 63 - RNA, 23 nt, Homo sapiens
ucuggcuccgugucuucacuccc

SEQ ID NO: 64 - RNA, 21 nt, Homo sapiens
ucagugcaugacagaacuugg

SEQ ID NO: 65 - RNA, 22 nt, Homo sapiens
uuaaugcuaaucgugauagggg

SEQ ID NO: 66 - RNA, 23 nt, Homo sapiens
aacauucauuguugucggugggu

SEQ ID NO: 67 - RNA, 24 nt, Homo sapiens
uuuggcaaugguagaacucacacu

SEQ ID NO: 68 - RNA, 22 nt, Homo sapiens
caaagaauucuccuuuugggcu

SEQ ID NO: 69 - RNA, 22 nt, Homo sapiens
ucgugucuuguguugcagccgg

SEQ ID NO: 70 - RNA, 23 nt, Homo sapiens
caacggaaucccaaaagcagcug

SEQ ID NO: 71 - RNA, 21 nt, Homo sapiens
aacuggccuacaaagucccag

SEQ ID NO: 72 - RNA, 22 nt, Homo sapiens
aacuggcccucaaagucccgcu

SEQ ID NO: 73 - RNA, 22 nt, Homo sapiens
uguaacagcaacuccaugugga

SEQ ID NO: 74 - RNA, 21 nt, Homo sapiens
uagcagcacagaaauauuggc

SEQ ID NO: 75 - RNA, 22 nt, Homo sapiens
uagguaguuucauguuguuggg

SEQ ID NO: 76 - RNA, 22 nt, Homo sapiens
uagguaguuuccuguuguuggg

SEQ ID NO: 77 - RNA, 22 nt, Homo sapiens
uucaccaccuucuccacccagc

SEQ ID NO: 78 - RNA, 23 nt, Homo sapiens
cccaguguucagacuaccuguuc

SEQ ID NO: 79 - RNA, 23 nt, Homo sapiens
cccaguguuuagacuaucuguuc

SEQ ID NO: 80 - RNA, 22 nt, Homo sapiens
uaacacugucugguaacgaugu

SEQ ID NO: 81 - RNA, 23 nt, Homo sapiens
uaauacugccugguaaugaugac

SEQ ID NO: 82 - RNA, 22 nt, Homo sapiens
uaauacugccggguaaugaugg

SEQ ID NO: 83 - RNA, 22 nt, Homo sapiens
gugaaauguuuaggaccacuag

SEQ ID NO: 84 - RNA, 22 nt, Homo sapiens
uucccuuugucauccuaugccu

SEQ ID NO: 85 - RNA, 22 nt, Homo sapiens
uccuucauuccaccggagucug

SEQ ID NO: 86 - RNA, 22 nt, Homo sapiens
acagcaggcacagacaggcagu

SEQ ID NO: 87 - RNA, 21 nt, Homo sapiens
augaccuaugaauugacagac

SEQ ID NO: 88 - RNA, 21 nt, Homo sapiens
uugugcuugaucuaaccaugu

SEQ ID NO: 89 - RNA, 24 nt, Homo sapiens
agcuacaucuggcuacugggucuc

SEQ ID NO: 90 - RNA, 21 nt, Homo sapiens
ugucaguuugucaaauacccc

SEQ ID NO: 91 - RNA, 21 nt, Homo sapiens
caagucacuagugguuccguu

SEQ ID NO: 92 - RNA, 23 nt, Homo sapiens
cagugcaauaguauugucaaagc

SEQ ID NO: 93 - RNA, 23 nt, Homo sapiens
cagugcaaugauauugucaaagc

SEQ ID NO: 94 - RNA, 22 nt, Homo sapiens
aaaagcuggguugagagggcga

SEQ ID NO: 95 - RNA, 22 nt, Homo sapiens
aaaagcuggguugagagggcaa

SEQ ID NO: 96 - RNA, 20 nt, Homo sapiens
aaaagcuggguugagagggu

SEQ ID NO: 97 - RNA, 19 nt, Homo sapiens
aaaagcuggguugagagga

SEQ ID NO: 98 - RNA, 23 nt, Homo sapiens
cgcauccccuagggcauuggugu

SEQ ID NO: 99 - RNA, 20 nt, Homo sapiens
acugccccaggugcugcugg

SEQ ID NO: 100 - RNA, 20 nt, Homo sapiens
ccucugggcccuuccuccag

SEQ ID NO: 101 - RNA, 22 nt, Homo sapiens
cuggcccucucugcccuuccgu

SEQ ID NO: 102 - RNA, 21 nt, Homo sapiens
gccccugggccuauccuagaa

SEQ ID NO: 103 - RNA, 22 nt, Homo sapiens
cuagguauggucccagggaucc

SEQ ID NO: 104 - RNA, 23 nt, Homo sapiens
ucaagagcaauaacgaaaaaugu

SEQ ID NO: 105 - RNA, 22 nt, Homo sapiens
uuaucagaaucuccagggguac

SEQ ID NO: 106 - RNA, 24 nt, Homo sapiens
aauccuuggaaccuaggugugagu

SEQ ID NO: 107 - RNA, 22 nt, Homo sapiens
aacacaccuauucaaggauuca

SEQ ID NO: 108 - RNA, 22 nt, Homo sapiens
uaaugccccuaaaaauccuuau

SEQ ID NO: 109 - RNA, 22 nt, Homo sapiens
uuuguucguucggcucgcguga

SEQ ID NO: 110 - RNA, 22 nt, Homo sapiens
uauacaagggcaagcucucugu

SEQ ID NO: 111 - RNA, 22 nt, Homo sapiens
agaucagaaggugauuguggcu

SEQ ID NO: 112 - RNA, 21 nt, Homo sapiens
uaguagaccguauagcguacg

SEQ ID NO: 113 - RNA, 22 nt, Homo sapiens
cuggacuuggagucagaaggcc

SEQ ID NO: 114 - RNA, 23 nt, Homo sapiens
aaugacacgaucacucccguuga

SEQ ID NO: 115 - RNA, 23 nt, Homo sapiens
ucuuggaguaggucauugggugg

SEQ ID NO: 116 - RNA, 23 nt, Homo sapiens
aaaccguuaccauuacugaguuu

SEQ ID NO: 117 - RNA, 22 nt, Homo sapiens
aacuguuugcagaggaaacuga

SEQ ID NO: 118 - RNA, 23 nt, Homo sapiens
ucacuccucuccucccgucuucu

SEQ ID NO: 119 - RNA, 22 nt, Homo sapiens
uccuguacugagcugccccgag

SEQ ID NO: 120 - RNA, 21 nt, Homo sapiens
cggggcagcucaguacaggau

SEQ ID NO: 121 - RNA, 22 nt, Homo sapiens
aaucguacagggucauccacuu

SEQ ID NO: 122 - RNA, 23 nt, Homo sapiens
aggaccugcgggacaagauucuu

SEQ ID NO: 123 - RNA, 21 nt, Homo sapiens
cagcagcacacugugguuugu

SEQ ID NO: 124 - RNA, 22 nt, Homo sapiens
aauccuuugucccugggugaga

SEQ ID NO: 125 - RNA, 22 nt, Homo sapiens
aaugcacccgggcaaggauucu

SEQ ID NO: 126 - RNA, 24 nt, Homo sapiens
uucuccaaaagaaagcacuuucug

SEQ ID NO: 127 - RNA, 23 nt, Homo sapiens
caucuggagguaagaagcacuuu

SEQ ID NO: 128 - RNA, 22 nt, Homo sapiens
caaagcgcuccccuuuagaggu

SEQ ID NO: 129 - RNA, 23 nt, Homo sapiens
caaagugccucccuuuagagugu

SEQ ID NO: 130 - RNA, 21 nt, Homo sapiens
gaaggcgcuucccuuuggagu

SEQ ID NO: 131 - RNA, 24 nt, Homo sapiens
cucuugagggaagcacuuucuguu

SEQ ID NO: 132 - RNA, 22 nt, Homo sapiens
caugccuugaguguaggaccgu

SEQ ID NO: 133 - RNA, 22 nt, Homo sapiens
ccucccacacccaaggcuugca

SEQ ID NO: 134 - RNA, 22 nt, Homo sapiens
ggggguccccggugcucggauc

SEQ ID NO: 135 - RNA, 22 nt, Homo sapiens
uccgagccugggucucccucuu

SEQ ID NO: 136 - RNA, 22 nt, Homo sapiens
uacccauugcauaucggaguug

SEQ ID NO: 137 - RNA, 22 nt, Homo sapiens
ugagguaguagguuguauaguu

SEQ ID NO: 138 - RNA, 22 nt, Homo sapiens
ugagguaguagguugugugguu

SEQ ID NO: 139 - RNA, 22 nt, Homo sapiens
ugagguaguagguuguaugguu

SEQ ID NO: 140 - RNA, 21 nt, Homo sapiens
agagguaguagguugcauagu

SEQ ID NO: 141 - RNA, 21 nt, Homo sapiens
ugagguaggagguuguauagu

SEQ ID NO: 142 - RNA, 22 nt, Homo sapiens
ugagguaguagauuguauaguu

SEQ ID NO: 143 - RNA, 22 nt, Homo sapiens
ugagguaguaguuuguacaguu

SEQ ID NO: 144 - RNA, 21 nt, Homo sapiens
ugagguaguaguuugugcugu

SEQ ID NO: 145 - RNA, 64 nt, Homo sapiens
uuauauuuauauucagaaaagaaacauuucaguaauuuauaauaaagagcacuauuuuuuaaug

SEQ ID NO: 146 - RNA, 57 nt, Mus musculus
auuuauauuuauaaaacauuucaguaauuuauaauaaaagagcacuauuuuuuaaug

SEQ ID NO: 147 - RNA, 57 nt, Rattus norvegicus
uauuuauauuuauaaaacauuucaguaauuuauaauaaagagcacuauuuuuuaaug

SEQ ID NO: 148 - RNA, 54 nt, Canis familiaris
uuauauuuauaaaacauuucaguaauuuauaauaaagagcacuauuuuuuaaug

SEQ ID NO: 149 - RNA, 21 nt, Homo sapiens
ugaaauguuuaggaccacuag

SEQ ID NO: 150 - RNA, 12 nt, Homo sapiens
gaaacauuucag

(19) United States
(12) Patent Application Publication
(10) Pub. No.: US 2010/0202973 A1
(43) Pub. Date: Aug. 12, 2010

(54) MICRORNA MOLECULES ASSOCIATED WITH INFLAMMATORY SKIN DISORDERS

(75) Inventors: Andor Pivarcsi, Stockholm (SE); Eniko Sonkoly, Stockholm (SE)

Correspondence Address: KLARQUIST SPARKMAN, LLP, 121 SW SALMON STREET, SUITE 1600, PORTLAND, OR, 97204, US

(73) Assignee: Karolinska Institutet Innovations AB

(21) Appl. No.: 12/600,744

(22) PCT Filed: May 13, 2008
(86) PCT No.: PCT/IB2008/001877

§ 371 Date: Nov. 18, 2009

Related U.S. Application Data
(60) Provisional application No. 60/930,702, filed on May 18, 2007.

(51) Int. Cl. A61K 49/00; C12Q 1/68; A61K 31/7105; G01N 33/53; A61P 17/06

(52) U.S. Cl. 424/9.2; 435/6; 514/44 A; 435/7.21

(57) ABSTRACT

This invention relates to the finding that inflammatory skin disorders, such as psoriasis and atopic eczema, are characterized by changes in the expression of specific microRNA molecules (miRNAs). These miRNAs may therefore be useful as biomarkers for inflammatory skin disorders as well as therapeutic targets. Methods of diagnosis and treatment of inflammatory skin disorders are provided, as well as methods of screening for therapeutic compounds.

This invention relates to microRNA molecules (miRNAs) which are associated with inflammatory skin disorders, such as psoriasis and atopic eczema.

Psoriasis and atopic eczema are the two most common chronic inflammatory skin diseases. Each disease affects 1-3% of the adult population worldwide and have a great negative impact on the patients' quality of life (Lebwohl 2003; Leung, Boguniewicz et al. 2004). A complex interplay of genetic and environmental factors together with immunoregulatory abnormalities is thought to play a critical role in the pathogenesis of both diseases. Although psoriasis and atopic eczema share several common features, the clinical characteristics and the so far identified molecular abnormalities are different. Keratinocytes and infiltrating immune cells play a cooperative role in these diseases; however the exact molecular mechanisms and the complex interactions among resident skin cells and infiltrating immunocytes are still not completely understood. Investigations analyzing the molecular background of psoriasis and atopic eczema have identified many disease-associated genes and proteins with aberrant expression (Nomura, Gao et al. 2003; Zhou, Krueger et al. 2003; Carlen, Sanchez et al. 2005), however, the underlying regulatory networks responsible for these gene expression alterations have not been fully characterized.

The present inventors have discovered that lesional skin from patients with inflammatory skin disorders such as psoriasis and atopic eczema is characterized by specific, non-random miRNA expression profiles which differ from the miRNA expression profile of healthy skin. This indicates that miRNAs represent a previously unreported, epigenetic mechanism in the pathogenesis of chronic skin inflammation and may be useful in the diagnosis and therapy of inflammatory skin disorders.

One aspect of the invention provides a method of diagnosing an inflammatory skin disorder in an individual comprising; determining the expression of one or more of the miRNAs in a sample of skin cells obtained from the individual,
wherein a change in expression of the one or more miRNAs in the sample relative to controls is indicative that the individual has an inflammatory skin disorder.

A method of diagnosing an inflammatory skin disorder in an individual may for example, comprise;
determining the expression of one or more of the miRNAs selected from the group consisting of let-7a, let-7b, let-7c, let-7d, let-7e, let-7f, let-7g, let-71, miR-1, miR-9, miR-10a, miR-10b, miR-15a, miR-15b, miR-16, miR-17-5p, miR-20a, miR-21, miR-22, miR-23a, miR-23b, miR-24, miR-26a, miR-26b, miR-27a, miR-27b, miR-29a, miR-29c, miR-30a-5p, miR-30a-3p, miR-30b, miR-30c, miR-30d, miR-30e-5p, miR-30e-3p, miR-31, miR-92, miR-93, miR-95, miR-99a. miR-99b, miR-100, miR-101, miR-106a, miR-106b, miR-107, miR-122a, miR-125a, miR-125b, miR-126, miR-127, miR-130a, miR-130b, miR-132, miR-133a, miR-133b, miR-135b, miR-140, miR-141, miR-142-3p, miR-142-5p, miR-143, miR-145, miR-146a, miR-146b, miR-148a, miR-148b, miR-149, miR-152, miR-155, miR-181d, miR-182, miR-186, miR-187, miR-191, miR-193a, miR-193b, miR-194, miR-195, miR-196a, miR-196b, miR-197, miR-199a, miR-199b, miR-200a, miR-200b, miR-200c, miR-203, miR-204, miR-205, miR-214, miR-218, miR-222, miR-223, miR-224, miR-301, miR-320, miR-324-5p, miR-326, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524* and miR-526b, miR-532, miR-615, miR-660, in a sample of skin cells obtained from the individual,
wherein a change in expression of the one or more miRNAs in the sample relative to controls is indicative that the individual has an inflammatory skin disorder.

In some embodiments, the expression of one or more of the miRNAs selected from the group consisting of let-7c, let-7d, let-7e, let-7i, miR-1, miR-10a, miR-10b, miR-15a, miR-15b, miR-16, miR-17-5p, miR-20a, miR-21, miR-22, miR-24, miR-27a, miR-27b, miR-29a, miR-30c, miR-30e-5p, miR-31, miR-99b, miR-100, miR-101, miR-106a, miR-106b, miR-107, miR-122a, miR-125b, miR-130a, miR-133a-133b, miR-133b, miR-141, miR-142-3p, miR-143, miR-146a, miR-146b, miR-155, miR-193a, miR-194, miR-197, miR-199a, miR-199b, miR-200a, miR-200b, miR-200c, miR-203, miR-205, miR-222, miR-223, miR-326, miR-335, miR-361, miR-365, miR-381, miR-422b, miR-451, miR-483, miR-487b, miR-492, miR-515-5p, miR-516-5p, miR-518b, miR-519d, miR-524* and miR-526b may be determined.

A microRNA (miRNA) is a ribonucleic acid molecule of about 19 to 23 nucleotides, usually 21 to 22 nucleotides. miRNA molecules are naturally produced by higher eukaryotic cells and reduce the expression of specific protein-coding genes by targeting cognate messenger RNA for translational repression. miRNAs are transcribed from non-protein-coding genes in the form of long precursor miRNA molecules. These precursors are processed by a dsRNA-specific nuclease in the cell nucleus into hairpin RNA molecules of 70-100 nucleotides. These hairpin RNA molecules are further processed in the cytosol by a second dsRNA specific nuclease to produce the mature 19 to 23 nucleotide miRNA (Ambros, 2003; Bartel and Chen, 2004; Czech 2006).

The sequences of preferred mature miRNAs described herein are set out in Table 2. The sequences of miRNA genes, precursors and mature miRNAs are also described in Lim L P, et al Science. 299:1540 (2003) and are publicly available from the miRNA Registry (miRBase) which is maintained by the Wellcome Trust Sanger Institute, Hinxton, UK. The miRBase database is described in Griffiths-Jones S. NAR, 2004, 32, D109-D111 and Griffiths-Jones S et al NAR, 2006, 34, D140-D144) and is available online at http://microrna.sanger.ac.uk/. To date, 342 human miRNAs have been registered in mirBase 8.0.

In the art, miRNAs are generally referred to by name. An assigned miRNA name refers unambiguously to a miRNA of a specific sequence. The annotation of miRNAs is described in Ambros V et al RNA, 2003, 9(3), 277-279.

Methods of diagnosing an inflammatory skin disorder as described herein may be useful in the diagnosis or prognosis of an inflammatory skin disorder in an individual; predicting the susceptibility, onset or likely severity of an inflammatory skin disorder in an individual; or in predicting the responsiveness of an individual to therapy. For example, a change in expression of a miRNA described herein relative to controls may be indicative of the onset of the inflammatory skin disorder or may be indicative that the individual is susceptible to or has a high risk of suffering from an inflammatory skin disorder relative to control members of the population.

The expression of one or more miRNAs selected from the group consisting of miR-20a, miR-146a, miR-20a, miR-31, miR-21, miR-17-5p, miR-193a, miR-146b, miR-146a, miR-222, miR-142-3p, let-71, miR-106a, miR-200a, miR-30e-5p, miR-203, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155 may be determined and/or the expression of one or more miRNAs selected from the group consisting of miR-93, miR-130b, miR-132, miR-135b, miR-142-5p, miR-155, miR-187, miR-205, miR-223, miR-224, miR-301, miR-324-5p, miR-362, miR-432, miR-425 and miR-501 may be determined. An increase in expression of one or more of these miRNAs in a sample relative to controls is indicative that the individual has an inflammatory skin disorder, for example psoriasis or atopic eczema.

In some preferred embodiments, the expression of one or more miRNAs selected from the group consisting of miR-20a, miR-17-5p, miR-21, miR-106a, let-71, miR-106b, miR-222, miR-422b, miR-130a, miR-193a, miR-106b, miR-199b, miR-199a, and miR-27b may be determined. An increase in expression of one or more of these miRNAs in a sample relative to controls is indicative that the individual has an inflammatory skin disorder, for example psoriasis or atopic eczema. More preferably, the expression of miR-21 may be determined.

The expression of one or more miRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c may be determined in the sample. A decrease in expression of one or more of these miRNAs relative to controls is indicative that the individual has an inflammatory skin disorder.

In some preferred embodiments, the expression of one or more miRNAs selected from the group consisting of miR-122a, miR-133a, miR-133b, miR-326, miR-125b, and miR-215 may be determined in the sample. A decrease in expression of one or more of these miRNAs relative to controls is indicative that the individual has an inflammatory skin disorder. More preferably, the expression of miR-125b may be determined.

Inflammatory skin disorders include conditions associated with chronic skin inflammation, such as atopic eczema, lupus erythematosus, lichen ruber, prurigo nodularis, psoriasiform disorders such as psoriasis, psoriasiform sarcoidosis, psoriasiform keratosis, psoriasiform-lichenoid dermatitis, pityriasis rubra pilaris, and glucagonoma syndrome.

In some embodiments, a change in the expression of one or more miRNAs is specifically indicative of psoriasis. Psoriasis is a chronic skin disorder which typically presents as well-demarcated erythematous scaling plaques most often symmetrically involving the elbows, knees, lower back, and buttocks. More than 90% of patients who present with psoriasis have symmetrical discrete plaques, but clinical manifestations can vary greatly (R S Stern, Psoriasis, Lancet 350 (1997), pp. 349-353).

An increase in expression of one or more miRNAs set out herein may be indicative of psoriasis. A method of diagnosing psoriasis in an individual may comprise the step of determining the expression of one or more miRNAs selected from the group consisting of miR-20a, miR-146a, miR-146b, miR-31, miR-146a, miR-20a, miR-200a, miR-17-5p, miR-30e-5p, miR-141, miR-203, miR-142-3p, miR-21, miR-106a, miR-487b, miR-15a, let-71, miR-222, miR-422b, miR-130a, miR-193a, miR-106b, miR-199b, miR-199a* and miR-27b and/or one or more miRNAs selected from the group consisting of miR-135b, miR-205, miR-155, miR-223, miR-93, miR-132, miR-425, miR-362, miR-324-5p, miR-224, miR-432 and miR-301 in a sample of skin cells obtained from the individual. An increase in expression of the one or more miRNAs in the sample cells relative to controls is indicative that the individual has psoriasis.

In some preferred embodiments, the expression of one or more miRNAs selected from the group consisting of miR-146a, miR-146b, miR-31, miR-20a, miR-200a, miR-30e-5p, miR-141, miR-203, miR-142-3p miR-487b, miR-15a may be determined. More preferably, the expression of miR-203 and/or miR-146a may be determined. An increase in expression of the one or more miRNAs in the sample cells relative to controls is indicative that the individual has psoriasis.

A decrease in the expression of one or more miRNAs set out herein may be indicative of psoriasis. A method of diagnosing psoriasis in an individual may comprise determining the expression of one or more miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194 and miR-526b and/or one or more miRNAs selected from the group consisting of miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in a sample of skin cells obtained from the individual.

In some preferred embodiments, the expression of one or more miRNAs selected from the group consisting of miR-99b, miR-197, miR-100, miR-381, miR-518b, miR-524, let-7e, miR-30c, miR-365, miR-10a and miR-22 may be determined.

A decrease in expression of the one or more miRNAs in the sample cells relative to controls is indicative that the individual has psoriasis.

In other embodiments, a change in the expression of one or more miRNAs set out herein is specifically indicative of atopic eczema. Atopic eczema is a chronic inflammatory skin disease associated with cutaneous hyperreactivity to environmental triggers that are innocuous to normal nonatopic individuals. The diagnosis of atopic eczema is based on the following constellation of clinical findings: pruritus, facial and extensor eczema in infants and children, flexural eczema in adults, and chronicity of the dermatitis (Leung, Boguniewicz et al. 2004).

An increase in expression of one or more miRNAs set out herein may be indicative of atopic eczema. A method of diagnosing atopic eczema in an individual may comprise;
determining the expression of one or more miRNAs selected from the group consisting of miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-203, miR-223, miR-199a*, miR-200c, miR-155, miR-16, miR-361, miR-205, miR-143, miR-422b, miR-200b and miR-15b and/or one or more miRNAs selected from the group consisting of miR-135b, miR-187, miR-142-5p, miR-501, miR-487b, miR-362, miR-301, miR-130b, and miR-146b.

In some preferred embodiments, the expression of one or more miRNAs selected from the group consisting of let-71, miR-29a, miR-222, miR-24, miR-193a, miR-199a and miR-27a may be determined.

An increase in expression of the one or more miRNAs relative to controls in indicative that the individual has atopic eczema.

A decrease in expression of one or more miRNAs set out herein may be indicative of atopic eczema. A method of diagnosing atopic eczema in an individual may comprise:
determining the expression of one or more miRNAs selected from the group consisting of miR-122a, miR-133a-133b, miR-326, miR-215, miR-483, miR-519d, miR-335, miR-133b, miR-515-5p, miR-194, miR-524* and miR-197 and/or one or more miRNAs selected from the group consisting of miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451.

In some preferred embodiments, the expression of one or more miRNAs selected from the group consisting of miR-483, miR-519d, miR-335 and miR-515-5p may be determined.

A decrease in expression of the one or more miRNAs relative to controls is indicative that the individual has atopic eczema.

The methods described above may comprise determining the expression of one or more, two or more, three or more, four or more, five or more, six or more, seven or more, eight or more, nine or more, ten or more, fifteen or more or twenty or more of the listed miRNAs.

The skilled person is readily able to employ suitable controls for use in the methods described herein. Suitable controls include cells from healthy (i.e. non-lesional) skin which is not affected by the inflammatory skin disorder. Control cells may be obtained from a different individual to the sample cells, for example a healthy individual not suffering from or susceptible to an inflammatory skin disorder.

A suitable sample of skin cells may be taken from a lesion or other site on the skin of the individual which displays one or more symptoms of an inflamatory disorder, such as inflammation, scaling and/or pruritus (i.e. a lesional sample). RNA may be isolated from the skin cells using methods well known in the art (see, e.g., Lagos-Quintana et al, Science 294:853-858 (2001); Grad et al, Mol Cell 11: 1253-1263 (2003); Mourelatos et al, Genes Dev 16:720-728 (2002); Lagos-Quintana et al, Curr Biol 12:735-739 (2002); Lagos-Quintana et al, RNA 9:175-179 (2003)).

The expression of a miRNA in a cell may be determined by measuring the amount of miRNA precursor or, more preferably the amount of mature miRNA, which is present in the cells.

The amount of miRNA in a cell may be conveniently measured by any convenient technique, including for example quantitative PCR, bead-based flow cytometry, microarrays, northern blotting, dot blotting, RNase protection assays, primer extension analysis, miRNA in situ hybridization, and Invader™ assays. Suitable techniques are described in Liu et al, (2004); Thomson et al. (2004); Babak et al. (2004), Chen, Ridzon et al. (2005); Castoldi, Schmidt et al. (2006) and Kim et al (2006); Kloosterman et al, Nature Methods, 3 (1), 27-29 (2006). Suitable reagents for miRNA in situ hybridization are commercially available (e.g. Exiqon A/S, Denmark).

In some embodiments, the expression of one or more miRNAs in a sample may be determined by microarray techniques. Microarrays generally comprise nucleic acid probes of different sequences immobilised in a predetermined arrangement on a solid support. Because different nucleic acid probes are immobilised at different locations on the support, the binding of a label which is observed at a particular location is indicative of specific binding to the nucleic acid probe immobilised at that location.

Microarrays may be synthesised using conventional techniques by synthesising nucleic acid probes and then attaching the probes to the support in a site-specific fashion, or by synthesising the nucleic acid probes in situ at predetermined locations on the support. Microarrays for use in the detection of human miRNAs are also commercially available (e.g. TaqMan® Human microRNA Array v1.0; Applied Biosystems, CA USA).

In use, a microarray is contacted with a sample under conditions that promote specific binding of miRNAs in the sample to one or more of the immobilised nucleic acid molecules on the microarray. The miRNAs in the sample bind to one or more different locations on the microarray, via the nucleic acid molecules immobilised at those locations to produce a particular binding pattern. This binding pattern can then be detected by any convenient technique. For example all nucleic acid molecules, including miRNA molecules, in the sample may be labelled with a suitable label, typically a fluorescent label, and the locations at which label is present on the microarray following exposure to the sample can be observed. Since the immobilised nucleic acid sequences have different predetermined locations on the microarray, the observed binding pattern is indicative of the presence and/or concentration of a particular miRNA in the sample. Techniques for detecting binding to microarrays are well known in the art (see for example, U.S. Pat. No. 5,763,870, U.S. Pat. No. 5,945,679 and U.S. Pat. No. 5,721,435).

A method of determining the expression of one or more miRNAs may, for example, comprise: a) contacting a sample with a microarray comprising immobilised probes for said one or more miRNAs under conditions sufficient for specific binding to occur between the miRNA and its corresponding immobilised probe; and b) interrogating the microarray to determined the presence or amount of binding of one or more miRNAs in the sample.

A suitable sample for contacting a microarray may be a sample of RNA isolated from skin cells obtained from the individual. Techniques for the preparation of RNA isolates from cells are well known in the art.

Conveniently, a Locked Nucleic Acid (LNA)-based miRNA microarray may be employed. Typically, total RNA is isolated from the sample skin cells, labelled and hybridized onto a microarray containing LNA (Locked Nucleic Acid)-modified probes for each known miRNA. The high affinity LNA technology provides the LNA Array with high sensitivity, high specificity and Tm-normalized probes. LNA microarrays are available commercially (e.g. miRCURY™, Exiqon).

In some embodiments, the expression of one or more miRNAs in a sample may be determined by bead-based flow cytometry methods such as FlexmiR™ (Exiqon A/S, Copenhagen) (Lu et al Nature 2005 435 834-838). This involves marking individual beads with fluorescence tags, each representing a single miRNA, and coupling the beads to probes that are complementary to miRNAs of interest. miRNAs are ligated to 5′ and 3′ adaptors, reverse-transcribed, amplified by PCR using a common biotinylated primer, hybridized to the capture beads, and stained with a suitable reagent such as streptavidin-phycoerythrin. The beads are then analyzed using a flow cytometer capable of measuring bead color (denoting miRNA identity) and phycoerythrin intensity (denoting miRNA abundance). Because hybridization takes place in solution, bead-based flow cytometry methods may allow more specific detection of closely related miRNAs than microarray techniques.

In some embodiments, the expression of one or more miRNAs in a sample may be determined by miRNA-specific quantitative real-time PCR. For this, total RNA is isolated from the skin biopsy, reverse transcribed using miRNA-specific stem-loop primers, and then amplified by real-time PCR, for example using TaqMan® probes. The assays target only mature microRNAs, not their precursors, ensuring biologically relevant results. Techniques for real-time PCR are well known in the art (Livak et al PCR Methods Appl (1995) 4 357-362) and reagents for use in such techniques are commercially available (e.g. Applied Biosystems, CA USA). Conveniently, real time PCR assays may be analysed on microarrays, for example on a micro-fluidic card. Suitable microarrays are commercially available (e.g. TaqMan® Human microRNA Array v1.0; Applied Biosystems, CA USA).

Methods of diagnosing an inflammatory skin disorder as described herein may also be useful in determining the responsiveness of an individual to a therapy for an inflammatory skin disorder, such as psoriasis or atopic eczema. A method of assessing the efficacy of a therapy for an inflammatory skin disorder in an individual or the responsiveness of an individual to an inflammatory skin disorder therapy may comprise:
determining the expression of one or more of the miRNAs set out above in one or more cells obtained from an individual subjected to a regimen of treatment with the inflammatory skin disorder therapy.

A control tissue sample may be obtained before the regimen of inflammatory skin disorder therapy is initiated. A change, for example, an increase or decrease in expression of one or more of the miRNAs set out above after initiation of the inflammatory skin disorder therapy regimen is indicative that the regimen normalises miRNA levels in a cell and is therefore efficacious for the treatment of the individual.

The absence of any change in the expression of the one or more of the miRNAs set out above after initiation of the regimen of inflammatory skin disorder therapy may be indicative that the regimen is not efficacious for the treatment of the individual.

The expression of the one or more of the miRNAs may be measured in samples obtained at one or more, two or more, or three or more time points during or after the treatment. The amount of change in the expression of the one or more of the miRNAs may be indicative of the level of responsiveness of the individual to the regimen.

Inflammatory skin disorder therapies are well-known in the art and may include topical or systemic treatments. Suitable treatments include anthralin, calcipotriene, betamethasone, accutane, hydrea, mycophenolate, mofetil, sulfasalazine, 6-thioguanine, dipropionate, tazarotene corticosteroids, cyclosporine, methotrexate, soriatone, IFNgamma, pimecrolimus, tacrolimus, biological agents such as alefacept, etanercept, adalimumab, efalizumab and infliximab and phototherapy, such as UV light therapy and chemophototherapy, for example using psoralen.

Other aspects of the invention relate to methods of treatment of an inflammatory skin disorder. A method of treatment of an inflammatory skin disorder, such as psoriasis or atopic eczema, in an individual may comprise;
increasing or reducing the expression or activity of one or more miRNAs selected from the group consisting of let-7a, let-7b, let-7c, let-7d, let-7e, let-7f, let-7g, let-71, miR-1, miR-9, miR-10a, miR-10b, miR-15a, miR-15b, miR-16, miR-17-5p, miR-20a, miR-21, miR-22, miR-23a, miR-23b, miR-24, miR-26a, miR-26b, miR-27a, miR-27b, miR-29a, miR-29c, miR-30a-5p, miR-30a-3p, miR-30b, miR-30c, miR-30d, miR-30e-5p, miR-30e-3p, miR-31, miR-92, miR-93, miR-95, miR-99a. miR-99b, miR-100, miR-101, miR-106a, miR-106b, miR-107, miR-122a, miR-125a, miR-125b, miR-126, miR-127, miR-130a, miR-130b, miR-132, miR-133a, miR-133b, miR-135b, miR-140, miR-141, miR-142-3p, miR-142-5p, miR-143, miR-145, miR-146a, miR-146b, miR-148a, miR-148b, miR-149, miR-152, miR-155, miR-181d, miR-182, miR-186, miR-187, miR-191, miR-193a, miR-193b, miR-194, miR-195, miR-196a, miR-196b, miR-197, miR-199a, miR-199b, miR-200a, miR-200b, miR-200c, miR-203, miR-204, miR-205, miR-214, miR-218, miR-222, miR-223, miR-224, miR-301, miR-320, miR-324-5p, miR-326, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524* and miR-526b, miR-532, miR-615 and miR-660 in cells of the individual.

The expression or activity of one or more miRNAs selected from the group consisting of let-7c, let-7d, let-7e, let-71, miR-1, miR-10a, miR-10b, miR-15a, miR-15b, miR-16, miR-17-5p, miR-20a, miR-21, miR-22, miR-24, miR-27a, miR-27b, miR-29a, miR-30c, miR-30e-5p, miR-31, miR-99b, miR-100, miR-101, miR-106a, miR-106b, miR-107, miR-122a, miR-125b, miR-130a, miR-133a-133b, miR-133b, miR-141, miR-142-3p, miR-143, miR-146a, miR-146b, miR-155, miR-193a, miR-194, miR-197, miR-199a, miR-199b, miR-200a, miR-200b, miR-200c, miR-203, miR-205, miR-222, miR-223, miR-326, miR-335, miR-361, miR-365, miR-381, miR-422b, miR-451, miR-483, miR-487b, miR-492, miR-515-5p, miR-516-5p, miR-518b, miR-519d, miR-524* and miR-526b may be increased or reduced in the cells.

For example, the expression or activity of miR-203 and/or miR-146a may be increased or reduced.

The expression or activity of one or more miRNAs selected from the group consisting of miR-20a, miR-146a, miR-20a, miR-31, miR-21, miR-17-5p, miR-193a, miR-146b, miR-146a, miR-222, miR-142-3p, let-71, miR-106a, miR-200a, miR-30e-5p, miR-203, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155; more preferably one or more miRNAs selected from the group consisting of miR-20a, miR-17-5p, miR-21, miR-106a, and miR-106b; in cells, preferably lesional skin cells, of the individual may be reduced. [symbol] For example, the expression or activity of miR-21 may be reduced.

A method of treatment of psoriasis in an individual may comprise;
increasing or reducing the expression or activity of one or more miRNAs selected from the group consisting of miR-20a, miR-146a, miR-146b, miR-31, miR-146a, miR-20a, miR-200a, miR-17-5p, miR-30e-5p, miR-141, miR-203, miR-142-3p, miR-21, miR-106a, miR-487b, miR-15a, let-71, miR-222, miR-422b, miR-130a, miR-193a, miR-106b, miR-199b, miR-199a*, miR-27b, miR-135b, miR-205, miR-155, miR-223, miR-93, miR-132, miR-425, miR-362, miR-324-5p, miR-224, miR-432, miR-301 miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194, miR-526b, miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in cells of the individual.

In some embodiments, a method of treatment of psoriasis may comprise;
reducing the expression or activity of one or more miRNAs selected from the group consisting of miR-20a, miR-146a, miR-146b, miR-31, miR-146a, miR-20a, miR-200a, miR-17-5p, miR-30e-5p, miR-141, miR-203, miR-142-3p, miR-21, miR-106a, miR-487b, miR-15a, let-71, miR-222, miR-422b, miR-130a, miR-193a, miR-106b, miR-199b, miR-199a* and miR-27b and/or one or more miRNAs selected from the group consisting of miR-135b, miR-205, miR-155, miR-223, miR-93, miR-132, miR-425, miR-362, miR-324-5p, miR-224, miR-432 and miR-301; more preferably one or more miRNAs selected from the group consisting of miR-146a, miR-146b, miR-31, miR-20a, miR-200a, miR-30e-5p, miR-141, miR-203 and miR-142-3p in cells, of the individual.

For example, the expression or activity of miR-203 and/or miR-146a may be reduced.

A method of treatment of atopic eczema in an individual may comprise;
increasing or reducing the expression or activity of one or more miRNAs selected from the group consisting of miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-203, miR-223, miR-199a*, miR-200c, miR-155, miR-16, miR-361, miR-205, miR-143, miR-422b, miR-200b, miR-15b, miR-135b, miR-187, miR-142-5p, miR-501, miR-487b, miR-362, miR-301, miR-130b, and miR-146b miR-122a, miR-133a-133b, miR-326, miR-215, miR-483, miR-519d, miR-335, miR-133b, miR-515-5p, miR-194, miR-524*, miR-197, miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451 in cells of the individual.

In some embodiments, a method of treatment of atopic eczema may comprise;
reducing the expression or activity of one or more miRNAs selected from the group consisting of miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-203, miR-223, miR-199a*, miR-200c, miR-155, miR-16, miR-361, miR-205, miR-143, miR-422b, miR-200b and miR-15b and/or one or more miRNAs selected from the group consisting of miR-135b, miR-187, miR-142-5p, miR-501, miR-487b, miR-362, miR-301, miR-130b, and miR-146b; more preferably one or more miRNAs selected from the group consisting of let-71, miR-29a, miR-222, miR-24, miR-193a, miR-199a and miR-27a in cells, of the individual.

The expression or activity of the one or more miRNAs may be increased or reduced in skin cells from the individual, preferably lesional skin cells.

Lesional skin cells are skin cells located within lesions at which the symptoms of the inflammatory disorder are displayed. Cells located at skin lesions may be of any skin cell type, including keratinocytes, melanocytes and dermal fibroblasts, or infiltrating immune cells, such as CD4+, CD8+ and CD4CD25high T cell subsets, NK cells, granulocytes, B cells, dendritic cells and mast cells. miRNA activity or expression in skin cells may be modulated by topical administration of therapeutic agents as described below.

The expression or activity of the one or more miRNAs may be increased or reduced in blood cells from the individual, preferably white blood cells, such as T cells. miRNA activity or expression in blood cells may be modulated by parental administration, for example intravenous injection, of therapeutic agents as described below.

The expression or activity of a target miRNA may be reduced by decreasing in total amount of the target miRNA in the cell or by decreasing the amount of the target miRNA which is present in the cell in an active form.

In some embodiments, the expression or activity of the target miRNA may be reduced by administering a therapeutically effective amount of a miRNA inhibitor to an individual in need thereof.

An inhibitor of a target miRNA is a compound which reduces or represses the activity or expression of the target miRNA. Preferably, the inhibitor has no effect or substantially no effect on non-target miRNAs. Suitable inhibitors may be readily designed by the skilled person from the sequence of the target miRNA. Sequences of target miRNAs are available from the miRNA Registry and are set out in Table 2.

Suitable inhibitors may include single or double stranded oligonucleotides which are able to bind to mature miRNA or its precursor forms and inhibit the activity of mature miRNA, prevent or inhibit its production or increase its rate of depletion. Suitable oligonucleotides may be oligodeoxyribonucleotides, oligoribonucleotides or modified oligonucleotides as described below

In some embodiments, the activity of a mature miRNA may be inhibited by the binding of a single stranded oligonucleotide which has a sequence which is sufficiently complementary to the sequence of the miRNA to hybridise to the target miRNA by Watson-Crick base-pairing. The use of such ‘antisense’ oligonucleotides is well-established in the art.

Oligonucleotides may be generated in vitro or ex vivo for administration or anti-sense RNA may be generated in vivo within cells in which inhibition is desired. Thus, double-stranded DNA may be placed under the control of a promoter in a “reverse orientation” such that transcription of the anti-sense strand of the DNA yields RNA which is complementary to the precursor miRNA. The complementary anti-sense RNA sequence may then bind with the target miRNA, inhibiting its cellular activity (see for example, Applied Antisense Oligonucleotide Technology C A. Stein (1998) Wiley & Sons).

A suitable oligonucleotide for inhibition of an miRNA may have about 10 to 30 nucleotides, preferably about 20 nucleotides e.g. 14-23 nucleotides, for example about 15, 16 or 17.

The construction of anti-sense sequences and their use is well known in the art and is described for example in Peyman and Ulman, Chemical Reviews, 90:543-584, (1990) and Crooke, Ann. Rev. Pharmacol. Toxicol. 32:329-376, (1992).

Nucleotides comprise a base portion, generally a heterocyclic base such as a purine or pyrimidine, which is covalently linked to a sugar group, typically a pentofuranosyl sugar, which further comprises a phosphate group. The phosphate group is generally linked to the 2′, 3′ or 5′ hydroxyl moiety of the sugar. The phosphate groups covalently link adjacent nucleotides to one another to form an oligonucleotide. Within the oligonucleotide structure, the phosphate groups are commonly referred to as forming the internucleotide backbone of the oligonucleotide. The normal linkage or backbone of RNA and DNA is a 3′ to 5′ phosphodiester linkage

Single-stranded oligonucleotides for the inhibition of miRNA activity may be chemically modified. Modified oligonucleotides are described in more detail below.

Examples of modified oligonucleotides which may be used to inhibit target miRNA molecules include LNA Knockdown probes, (Orom, Kauppinen et al. 2006), 2′-O-methyl modified RNA oligonucleotides (Cheng, Byrom et al. 2005), and “antagomirs” (Krutzfeldt, Rajewsky et al. 2005 Mattes et al 2007).

Antagomirs are chemically modified, single-stranded RNA analogues conjugated to cholesterol. An antagomir typically comprises at least 19 nucleotides which are complementary to the sequence of a target miRNA which allow hybridisation between the antagomir and the target miRNA, thereby inhibiting the activity of the miRNA target. Antagomirs can discriminate between single nucleotide mismatches of the targeted miRNA and have been shown to silence specific miRNAs in vivo (Krutzfeldt, Rajewsky et al. 2005). Antagomirs have also been shown to efficiently target miRNAs when injected locally into the mouse cortex (Krutzfeldt, Kuwajima et al. 2007).

Other useful inhibitors include oligonucleotides which cause inactivation or cleavage of mature miRNA or its precursor forms. Suitable oligonucleotides may be chemically modified, or have enzyme activity, which causes cleavage of a nucleic acid at a specific site—thus influencing activity of miRNAs. Examples include ribozymes, EDTA-tethered oligonucleotides, or covalently bound oligonucleotides, such as a psoralen or other cross-linking reagent-bound oligonucleotides. Background references for ribozymes include Kashani-Sabet and Scanlon, 1995, Cancer Gene Therapy, 2 (3): 213-223, and Mercola and Cohen, 1995, Cancer Gene Therapy, 2 (1), 47-59.

In some embodiments, the activity of a mature miRNA may be inhibited using a double-stranded oligonucleotide which comprises a sequence which is complementary to a target miRNA. A suitable double-stranded oligonucleotide may comprise about 10 to 30 nucleotides, preferably about 20 nucleotides e.g. 18-23 nucleotides. Techniques for inhibiting target miRNAs using double-stranded inhibitory oligonucleotides are known in the art (Soutschek, J. et al Nature 432, 173-178 (2004), Vermeulen, Robertson et al. 2007 and US20050182005).

Other useful inhibitors include double- or single-stranded DNA or double- or single-stranded RNA “aptamers” that bind to specific targets via interactions other than Watson-Crick base pairing. Suitable oligonucleotides (e.g., RNA oligonucleotides) that bind a specific miRNA can be generated using the techniques of SELEX (Tuerk, 1997, Methods Mol Biol 67, 2190). In this technique, a very large pool (10⁶-10⁹) of random sequence nucleic acids is bound to the target using conditions that cause a large amount of discrimination between molecules with high affinity and low affinity for binding the target. The bound molecules are separated from unbound, and the bound molecules are amplified by virtue of a specific nucleic acid sequence included at their termini and suitable amplification reagents. This process is reiterated several times until a relatively small number of molecules remain that possess high binding affinity for the target. These molecules can then be tested for their ability to modulate miRNA activity as described herein.

A modified oligonucleotide may contain one or more modified backbone linkages. Backbone linkages in a modified oligonucleotide may include, for example, non-phosphodiester linkages, such as phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogues of these, and those having inverted polarity wherein the adjacent pairs of nucleoside units are linked 3′-5′ to 5′-3′ or 2′-5′ to 5′-2′. Modified oligonucleotides may comprise linkages which lack phosphate groups and may comprise short chain alkyl or cycloalkyl internucleoside linkages, mixed heteroatom and alkyl or cycloalkyl internucleoside linkages, or one or more short chain heteroatomic or heterocyclic internucleoside linkages, for example morpholino; siloxane; sulfide, sulfoxide, sulfone; formacetyl; thioformacetyl; methylene formacetyl; thioformacetyl; alkene containing; sulfamate; methyleneimino; methylenehydrazino; sulfonate; sulfonamide; amide; or other linkages comprising N, O, S and/or CH₂ groups.

Suitable modified oligonucleotides may comprise phosphorothioate backbones or heteroatom backbones, and in particular —CH₂—NH—O—CH₂—, —CH₂—N(CH.sub.3)-O—CH₂—, —CH.sub.2-O—N(CH₃)—CH₂—, —CH₂—N(CH₃)—N(CH₃)—CH₂— and —O—N(CH₃)—CH₂—CH₂— [wherein the native phosphodiester backbone is represented as —O—P—O—CH₂—].

Modified oligonucleotides may also contain one or more substituted sugar moieties. Suitable sugar moieties may comprise one of the following at the 2′ position: OH; F; O-, S-, or N-alkyl; O-, S-, or N-alkenyl; O-, S- or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Particularly suitable are O[(CH₂)ₙO]ₘCH₃, O[CH₂)ₙO]ₘOCH₃, O[(CH₂)ₙO]ₘNH₂, O[(CH₂)ₙ]ₘCH₃, O[(CH₂)ₙO]ₘONH₂ and O[(CH₂)ₙO]ₘON(CH₂)ₙCH₃)]₂, where n and m are from 1 to about 10.

Modified sugar moieties may comprise one of the following at the 2′ position: C₁ to C₁₀ lower alkyl, substituted lower alkyl, alkaryl, aralkyl, O-alkaryl or O-aralkyl, SH, SCH₃, OCN, Cl, Br, CN, CF, OCF, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Suitable modifications include 2′-methoxyethoxy (2′-O—CH₂CH₂OCH₃, also known as 2′-O-(2-methoxyethyl) or 2′-MOE) (Martin et al., Helv. Chim. Acta, 1995, 78, 486 504) i.e. an alkoxyalkoxy group, 2′-dimethylaminooxyethoxy, i.e., a O(CH₂)₂ON(CH₃)₂ group, also known as 2′-DMAOE, 2′-methoxy (2′-O—CH₃), 2′-aminopropoxy (2′-OCH₂CH₂CH₂NH₂) and 2′-fluoro (2′-F). Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide.

Modified oligonucleotides may also contain one or more sugar mimetics instead of a pentofuranosyl sugar. Suitable sugar mimetics include cyclobutyl moieties, azido-ribose, carbocyclic sugar analogues a-anomeric sugars; epimeric sugars such as arabinose, xyloses or lyxoses, pyranose sugars, furanose sugars, and sedoheptulose.

Modified oligonucleotides may also include base modifications or substitutions. Modified nucleotide bases can be used instead of or in addition to the naturally occurring bases i.e. the purine bases adenine (A) and guanine (G), and the pyrimidine bases thymine (T), cytosine (C) and uracil (U). For example, modified bases may increase the stability of the molecule. Modified bases known in the art include alkylated purines and pyrimidines, acylated purines and pyrimidines, and other heterocycles. These classes of pyrimidines and purines are known in the art and include pseudoisocytosine, N4,N4-ethanocytosine, 8-hydroxy-N-6-methyladenine, 4-acetylcytosine, 5-(carboxyhydroxylmethyl)uracil, 5 fluorouracil, 5-bromouracil, 5-carboxymethylaminomethyl-2-thiouracil, 5-carboxymethylaminomethyl uracil, dihydrouracil, inosine, N6-isopentyl-adenine, 1-methyladenine, 1-methylpseudouracil, 1-methylguanine, 2,2-dimethylguanine, 2-methyladenine, 2-methylguanine, 3-methylcytosine, 5-methylcytosine, N6-methyladenine, 7-methylguanine, 5-methylaminomethyl uracil, 5-methoxy amino methyl-2-thiouracil, -D-mannosylqueosine, 5-methoxycarbonylmethyluracil, 5-methoxyuracil, 2 methylthio-N-6-isopentenyladenine, uracil-5-oxyacetic acid methyl ester, psueouracil, 2-thiocytosine, 5-methyl-2 thiouracil, 2-thiouracil, 4-thiouracil, 5-methyluracil, N-uracil-5-oxyacetic acid methylester, uracil 5-oxyacetic acid, queosine, 2-thiocytosine, 5-propyluracil, 5-propylcytosine, 5-ethyluracil, 5-ethylcytosine, 5-butyluracil, 5-pentyluracil, 5-pentylcytosine, and 2,6,diaminopurine, methylpsuedouracil, 1-methylguanine and 1-methylcytosine.

In some embodiments, both the sugar and the backbone linkage of one or more, preferably all of the nucleotides in a modified oligonucleotide may be replaced with non-natural groups. The bases are maintained for hybridization with the target miRNA. Suitable modified oligonucleotides may include peptide nucleic acids (PNA). In PNA, the oligonucleotide sugar-backbone is replaced with an amide containing backbone, in particular an aminoethylglycine backbone. The bases are retained and are bound directly or indirectly to aza-nitrogen atoms of the amide portion of the backbone.

Modified oligonucleotides may be chemically linked to one or more moieties or groups which enhance the activity, cellular distribution or cellular uptake of the oligonucleotide. Suitable moieties include lipid moieties such as cholesterol, cholic acid, a thioether, e.g., hexyl-S-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety.

miRNA inhibitors may be transferred into the cell using a variety of techniques well known in the art. For example, oligonucleotide inhibitors can be delivered into the cytoplasm without specific modification. Alternatively, they may be delivered by the use of liposomes which fuse with the cellular membrane or are endocytosed, i.e. by employing ligands such as antibodies which are attached to the liposome or directly to the oligonucleotide and which bind to surface membrane protein receptors of the cell, resulting in endocytosis. Alternatively, the cells may be permeabilized to enhance transport of the oligonucleotides into the cell, without injuring the host cells or a DNA binding protein, e.g., HBGF-1, which transports oligonucleotides into a cell may be employed.

In other embodiments, a method of treatment of an inflammatory skin disorder in an individual may comprise;
increasing the amount or activity of one or more miRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c; more preferably one or more miRNAs selected from the group consisting of miR-122a, miR-133b, miR-133a-133b, miR-326, miR-125b, and miR-215 in cells of the individual.

In some preferred embodiments, the level or activity of miR-125b may be increased.

In some embodiments, a method of treatment of psoriasis in an individual may comprise;
increasing the amount or activity of one or more miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194 and miR-526b and/or one or more miRNAs selected from the group consisting of miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191; more preferably one or more miRNAs selected from the group consisting of miR-99b, miR-197, miR-100, miR-381, miR-518b, miR-524, let-7e, miR-30c, miR-365, miR-10a and miR-22 in cells of the individual.

In some embodiments, a method of treatment of atopic eczema individual may comprise;
increasing the amount or activity of one or more miRNAs selected from the group consisting of miR-122a, miR-133a-133b, miR-326, miR-215, miR-483, miR-519d, miR-335, miR-133b, miR-515-5p, miR-194, miR-524* and miR-197 and/or one or more miRNAs selected from the group consisting of miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451; more preferably one or more miRNAs selected from the group consisting of miR-483, miR-519d, miR-335 and miR-515-5p in cells of the individual.

The expression or activity of a target miRNA may be increased by administering to an individual in need thereof a therapeutically effective amount of;
(i) the target miRNA or a precursor thereof,
(ii) a nucleic acid encoding the target miRNA or a precursor thereof,
(iii) an analogue, derivative or modified form of the target miRNA which retains activity.

Nucleic acid sequences encoding a target miRNA or a target miRNA precursor may be comprised within a vector. Suitable vectors can be chosen or constructed, containing appropriate regulatory sequences which will drive transcription in the target cell, including promoter sequences, terminator fragments, polyadenylation sequences, enhancer sequences, marker genes and other sequences as appropriate.

A vector may comprise a selectable marker to facilitate selection of the transgenes under an appropriate promoter. For further details see, for example, Molecular Cloning: a Laboratory Manual: 3rd edition, Sambrook & Russell, 2001, Cold Spring Harbor Laboratory Press.

Many known techniques and protocols for manipulation of nucleic acid, for example in preparation of nucleic acid constructs, mutagenesis, sequencing, introduction of DNA into cells and gene expression, and analysis of proteins, are described in detail in Protocols in Molecular Biology, Second Edition, Ausubel at al. eds. John Wiley & Sons, 1992.

A nucleic acid vector may be introduced into a host cell, for example a lesional skin cell. Suitable techniques for transporting the constructor vector into the cell are well known in the art and include calcium phosphate transfection, DEAE-Dextran, electroporation, liposome-mediated transfection and transduction using retrovirus or other virus, e.g. vaccinia or lentivirus.

The particular choice of a transformation technology will be determined by its efficiency to transform the particular host cells employed as well as the experience and preference of the operator with a particular methodology of choice.

An analogue, derivative or modified form of a miRNA retains the biological activity of the mature miRNA (i.e. a miRNA agonist) and may be a oligoribonucleotide or oligodeoxyribonucleotide with one or more modifications which improve the stability, transport or other pharmacological properties. Suitable modifications include modifications to the backbone linkages, bases or sugar moieties of one or more of the constituent nucleotides and are described in more detail above.

The term “treatment” in the context of treating a inflammatory skin disorder, pertains generally to treatment and therapy, whether of a human or an animal (e.g. in veterinary applications), in which some desired therapeutic effect is achieved, for example, the inhibition of the progress of the disorder, and includes a reduction in the rate of progress, a halt in the rate of progress, amelioration of the disorder, and cure of the disorder. Treatment as a prophylactic measure (i.e. prophylaxis) is also included.

While it is possible for an active compound such as an miRNA agonist or antagonist as described above, to be administered alone, it is preferable to present it as a pharmaceutical composition (e.g., formulation) comprising at least one active compound, as defined above, together with one or more pharmaceutically acceptable carriers, adjuvants, excipients, diluents, fillers, buffers, stabilisers, preservatives, lubricants, or other materials well known to those skilled in the art and optionally other therapeutic or prophylactic agents.

Pharmaceutical compositions comprising a miRNA agonist or antagonist as defined above, for example, admixed or formulated together with one or more pharmaceutically acceptable carriers, excipients, buffers, adjuvants, stabilisers, or other materials, as described herein, may be used in the methods described herein.

The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well-known in the art of pharmacy. Such methods include the step of bringing the active compound into association with a carrier which may constitute one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

The miRNA agonist or antagonist (s) or pharmaceutical composition comprising the miRNA agonist or antagonist (s) may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

In preferred embodiments, an active compound is administered directly at the site of action by topical administration to lesional skin cells.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for parenteral administration include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example, from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use.

It will be appreciated that appropriate dosages of the miRNA agonist or antagonist(s), and compositions comprising the miRNA agonist or antagonist(s), can vary from patient to patient. Determining the optimal dosage will generally involve the balancing of the level of diagnostic benefit against any risk or deleterious side effects of the administration. The selected dosage level will depend on a variety of factors including, but not limited to, the route of administration, the time of administration, the rate of excretion of the miRNA agonist or antagonist(s), the amount of contrast required, other drugs, compounds, and/or materials used in combination, and the age, sex, weight, condition, general health, and prior medical history of the patient. The amount of miRNA agonist or antagonist(s) and route of administration will ultimately be at the discretion of the physician, although generally the dosage will be to achieve concentrations of the miRNA agonist or antagonist (s) at a lesion site without causing substantial harmful or deleterious side-effects.

Administration in vivo can be effected in one dose, continuously or intermittently (e.g., in divided doses at appropriate intervals). Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the physician. Other aspects of the invention relate to screening for compounds useful in the treatment of inflammatory skin disorders.

A method of screening for a compound useful in the treatment of an inflammatory skin disorder, such as psoriasis or atopic eczema, may comprise;
contacting a cell with a test compound and;
determining the activity or expression of one or more miRNAs selected from the group consisting of let-7a, let-7b, let-7c, let-7d, let-7e, let-7f, let-7g, let-71, miR-1, miR-9, miR-10a, miR-10b, miR-15a, miR-15b, miR-16, miR-17-5p, miR-20a, miR-21, miR-22, miR-23a, miR-23b, miR-24, miR-26a, miR-26b, miR-27a, miR-27b, miR-29a, miR-29c, miR-30a-5p, miR-30a-3p, miR-30b, miR-30c, miR-30d, miR-30e-5p, miR-30e-3p, miR-31, miR-92, miR-93, miR-95, miR-99a. miR-99b, miR-100, miR-101, miR-106a, miR-106b, miR-107, miR-122a, miR-125a, miR-125b, miR-126, miR-127, miR-130a, miR-130b, miR-132, miR-133a, miR-133b, miR-135b, miR-140, miR-141, miR-142-3p, miR-142-5p, miR-143, miR-145, miR-146a, miR-146b, miR-148a, miR-148b, miR-149, miR-152, miR-155, miR-181d, miR-182, miR-186, miR-187, miR-191, miR-193a, miR-193b, miR-194, miR-195, miR-196a, miR-196b, miR-197, miR-199a, miR-199b, miR-200a, miR-200b, miR-200c, miR-203, miR-204, miR-205, miR-214, miR-218, miR-222, miR-223, miR-224, miR-301, miR-320, miR-324-5p, miR-326, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524*, miR-526b, miR-532, miR-615, and miR-660 relative to controls,
wherein an increase or decrease in activity or expression in the presence of the test compound is indicative that the compound is useful in the treatment of an inflammatory skin disorder.

The in activity or expression of one or more miRNAs selected from the group consisting of let-7c, let-7d, let-7e, let-71, miR-1, miR-10a, miR-10b, miR-15a, miR-15b, miR-16, miR-17-5p, miR-20a, miR-21, miR-22, miR-24, miR-27a, miR-27b, miR-29a, miR-30c, miR-30e-5p, miR-31, miR-99b, miR-100, miR-101, miR-106a, miR-106b, miR-107, miR-122a, miR-125b, miR-130a, miR-133a-133b, miR-133b, miR-141, miR-142-3p, miR-143, miR-146a, miR-146b, miR-155, miR-193a, miR-194, miR-197, miR-199a, miR-199b, miR-200a, miR-200b, miR-200c, miR-203, miR-205, miR-222, miR-223, miR-326, miR-335, miR-361, miR-365, miR-381, miR-422b, miR-451, miR-483, miR-487b, miR-492, miR-515-5p, miR-516-5p, miR-518b, miR-519d, miR-524* and miR-526b may be determined relative to controls.

In some embodiments, in activity or expression of one or more microRNAs selected from the group consisting of one or more of the miRNAs selected from the group consisting of miR-20a, miR-146a, miR-20a, miR-31, miR-21, miR-17-5p, miR-193a, miR-146b, miR-146a, miR-222, miR-142-3p, let-71, miR-106a, miR-200a, miR-30e-5p, miR-203, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155 may be determined in the cell,
wherein a decrease in activity or expression in the presence of the test compound relative to its absence is indicative that the compound is useful in the treatment of an inflammatory skin disorder.

For example, expression of miR-21 may be determined.

In some embodiments, in activity or expression of one or more microRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c may be determined in the cell,
wherein an increase in activity or expression of the one or more microRNAs in the presence of the test compound relative to its absence is indicative that the compound is useful in the treatment of an inflammatory skin disorder.

[symbol]

For example, expression or activity of miR-125b may be determined.

A method of screening for a compound useful in the treatment of psoriasis may comprise;
contacting a cell with a test compound and;
determining the in activity or expression of one or more miRNAs selected from the group consisting of miR-20a, miR-146a, miR-146b, miR-31, miR-146a, miR-20a, miR-200a, miR-17-5p, miR-30e-5p, miR-141, miR-203, miR-142-3p, miR-21, miR-106a, miR-487b, miR-15a, let-71, miR-222, miR-422b, miR-130a, miR-193a, miR-106b, miR-199b, miR-199a* and miR-27b and/or one or more miRNAs selected from the group consisting of miR-135b, miR-205, miR-155, miR-223, miR-93, miR-132, miR-425, miR-362, miR-324-5p, miR-224, miR-432 and miR-301; in the cell,
wherein a decrease in activity or expression in the presence of the test compound relative to its absence is indicative that the compound is useful in the treatment of psoriasis.

[symbol]

For example, the activity or expression of miR-203 and/or miR-146a may be determined in the cell.

[symbol]

A method of screening for a compound useful in the treatment of psoriasis may comprise
contacting a cell with a test compound and;
determining the activity or expression of one or more miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194 and miR-526b and/or one or more miRNAs selected from the group consisting of miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, m±R-383, let-7a and miR-191; in the cell,
wherein an increase in activity or expression in the presence of the test compound relative to its absence is indicative that the compound is useful in the treatment of psoriasis.

[symbol]

A method of screening for a compound useful in the treatment of atopic eczema may comprise;
contacting a cell with a test compound and;
determining the activity or expression of one or more microRNAs selected from the group consisting of miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-203, miR-223, miR-199a*, miR-200c, miR-155, miR-16, miR-361, miR-205, miR-143, miR-422b, miR-200b and miR-15b and/or one or more miRNAs selected from the group consisting of miR-135b, miR-187, miR-142-5p, miR-501, miR-487b, miR-362, miR-301, miR-130b, and miR-146b,
wherein a decrease in activity or expression in the presence of the test compound relative to its absence is indicative that the compound is useful in the treatment of atopic eczema.

For example, the activity or expression of one or more miRNAs selected from the group consisting of miR-483, miR-519d, miR-335 and miR-515-5p.

A method of screening for a compound useful in the treatment of atopic eczema may comprise;
determining the activity or expression of one or more miRNAs selected from the group consisting of miR-122a, miR-133a-133b, miR-326, miR-215, miR-483, miR-519d, miR-335, miR-133b, miR-515-5p, miR-194, miR-524* and miR-197 and/or one or more miRNAs selected from the group consisting of miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451; in the cell,
wherein an increase in activity or expression in the presence of the test compound relative to its absence is indicative that the compound is useful in the treatment of treatment of atopic eczema.

Techniques for determining the amount of expression of a target miRNA in a cell are described in more detail above.

The cell is contacted with the test compound in vitro and may be an isolated cell, for example a cell from a cultured cell line or may be comprised in or obtained from a tissue sample which is obtained from an individual.

Suitable cells for use in the present methods may be higher eukaryotic cells, preferably mammalian cells, such as human cells. In preferred embodiments, the cell may be an a T cell or a human skin cell, for example a keratinocyte, melanocyte or dermal fibroblast, or an infiltrating immune cell, such as CD4+, CD8+ and CD4CD25high T cell subset, NK cell, granulocyte, B cell, dendritic cell or mast cell.

The precise format for performing the methods described herein may be varied by those of skill in the art using routine skill and knowledge.

Compounds which may be screened using the methods described herein may be natural or synthetic chemical compounds used in drug screening programmes. Extracts of plants, microbes or other organisms which contain several characterised or uncharacterised components may also be used.

Combinatorial library technology provides an efficient way of testing a potentially vast number of different compounds for ability to modulate an interaction. Such libraries and their use are known in the art, for all manner of natural products, small molecules and peptides, among others. The use of peptide libraries may be preferred in certain circumstances.

In some embodiments, the test compound may be an analogue, variant or derivative of a target miRNA as described above.

The amount of test compound or compound which may be added to a method of the invention will normally be determined by serial dilution experiments. Typically, from about 0.001 nM to 1 mM or more of putative inhibitor compound may be used, for example from 0.01 nM to 100 μM, e.g. 0.1 to 50 μM, such as about 10 μM.

In some embodiments, a method may comprise identifying the test compound as a miRNA inhibitor or antagonist as described above. Such a compound may, for example, be useful in reducing the expression and/or activity of the target miRNA, for example in the treatment of an inflammatory skin disorder, as described herein.

In other embodiments, a method may comprise identifying the test compound as an agonist (i.e. a promoter or enhancer) of a miRNA described above. Such a compound may, for example, be useful in increasing the expression and/or activity of the target miRNA, for example in the treatment of an inflammatory skin disorder, as described herein.

A test compound identified using one or more initial screens as having ability to modulate the expression and/or activity of one or more target miRNAs, may be assessed further using one or more secondary screens. A secondary screen may, for example, involve testing for a biological function such as an effect on skin lesions in an animal model of an inflammatory skin disorder.

The test compound may be isolated and/or purified or alternatively, it may be synthesised using conventional techniques of recombinant expression or chemical synthesis. Furthermore, it may be manufactured and/or used in preparation, i.e. manufacture or formulation, of a composition such as a medicament, pharmaceutical composition or drug. These may be administered to individuals for the treatment of a inflammatory skin disorder. Methods of the invention may thus comprise formulating the test compound in a pharmaceutical composition with a pharmaceutically acceptable excipient, vehicle or carrier for therapeutic application, as discussed further below.

Following identification of a compound which inhibits the expression or activity of a target miRNA described herein and which may therefore be useful in treating an inflammatory skin disorder, a method may further comprise modifying the compound to optimise the pharmaceutical properties thereof.

The modification of a ‘lead’ compound identified as biologically active is a known approach to the development of pharmaceuticals and may be desirable where the active compound is difficult or expensive to synthesise or where it is unsuitable for a particular method of administration, e.g. peptides are not well suited as active agents for oral compositions as they tend to be quickly degraded by proteases in the alimentary canal. Modification of a known active compound (for example, to produce a mimetic) may be used to avoid randomly screening large number of molecules for a target property.

Modification of a ‘lead’ compound to optimise its pharmaceutical properties commonly comprises several steps. Firstly, the particular parts of the compound that are critical and/or important in determining the target property are determined. In the case of a peptide, this can be done by systematically varying the amino acid residues in the peptide, e.g. by substituting each residue in turn. These parts or residues constituting the active region of the compound are known as its “pharmacophore”.

Once the pharmacophore has been found, its structure is modelled according its physical properties, e.g. stereochemistry, bonding, size and/or charge, using data from a range of sources, e.g. spectroscopic techniques, X-ray diffraction data and NMR.

Computational analysis, similarity mapping (which models the charge and/or volume of a pharmacophore, rather than the bonding between atoms) and other techniques can be used in this modelling process.

In a variant of this approach, the three-dimensional structure of the compound which modulates the expression and/or activity of a target miRNA described herein is modelled. This can be especially useful where the compound changes conformation, allowing the model to take account of this in the optimisation of the lead compound.

A template molecule is then selected, onto which chemical groups that mimic the pharmacophore can be grafted. The template molecule and the chemical groups grafted on to it can conveniently be selected so that the modified compound is easy to synthesise, is likely to be pharmacologically acceptable, and does not degrade in vivo, while retaining the biological activity of the lead compound. The modified compounds found by this approach can then be screened to see whether they have the target property, or to what extent they exhibit it. Modified compounds include mimetics of the lead compound.

Further optimisation or modification can then be carried out to arrive at one or more final compounds for in vivo or clinical testing.

As described above, a compound identified and/or obtained using the present methods may be formulated into a pharmaceutical composition.

Pharmaceutical compositions are described in more detail above. Various further aspects and embodiments of the present invention will be apparent to those skilled in the art in view of the present disclosure.

All documents mentioned in this specification and the miRNA registry entries for all the miRNAs mentioned are incorporated herein by reference in their entirety.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

Certain aspects and embodiments of the invention will now be illustrated by way of example and with reference to the figures and tables described below.

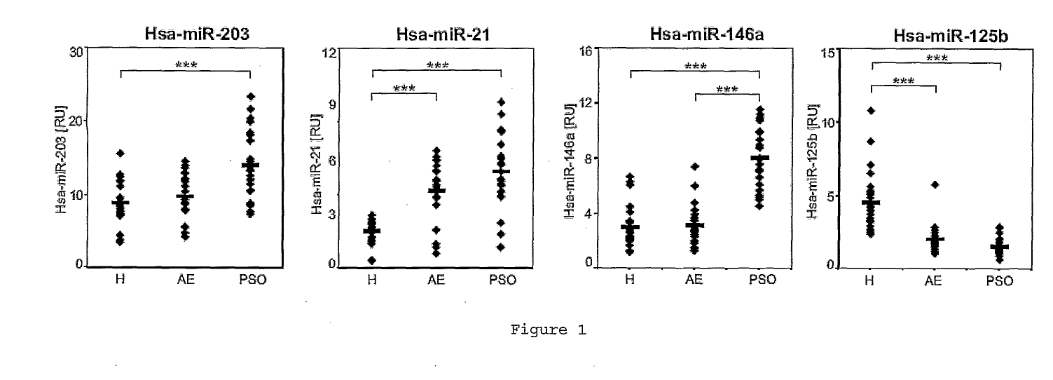

FIG. 1 shows that miRNAs are differentially expressed between chronic inflammatory skin diseases and healthy skin. The expressions of the functionally active, mature forms of four miRNAs were analyzed using quantitative real-time PCR in the skin of 26 healthy individuals, and lesional skin samples of 20 patients with atopic eczema and 25 patients with psoriasis. The results for individual patients and mean are shown. Data are expressed in relative units compared to U48 RNA. ***p<0.001.

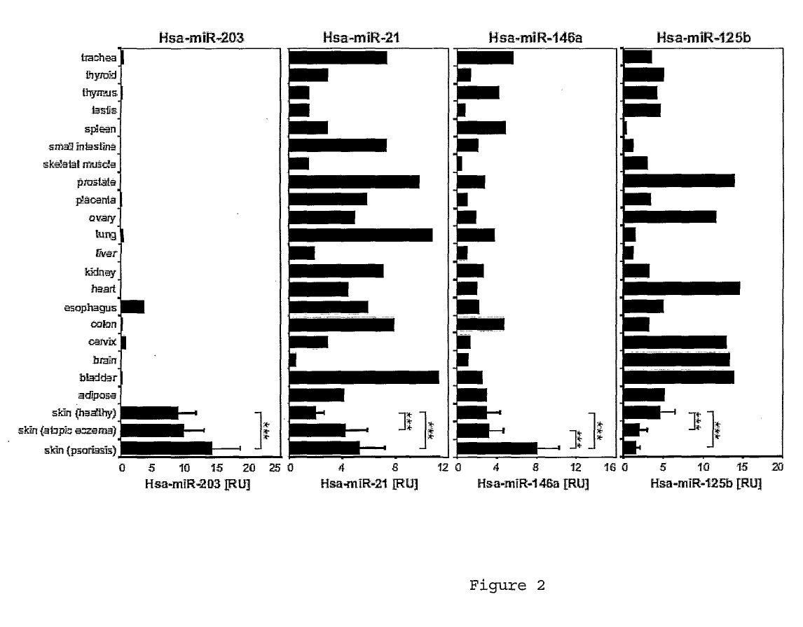

FIG. 2 shows the expression of miR-203, miR-146a, miR-21 and miR-125b in human organs. The expressions of the functionally active, mature forms of miR-203, miR-21, miR-146a and miR-125b were analyzed using quantitative real-time PCR in 20 healthy organs and tissues (each a pool of three donors) as well as in healthy skin (n=26), lesional atopic eczema (n=20) and psoriasis (n=25) skin samples. Error bars represent the standard error of the mean. Data are expressed in relative units compared to U48 RNA. ***_{p<}0.001.

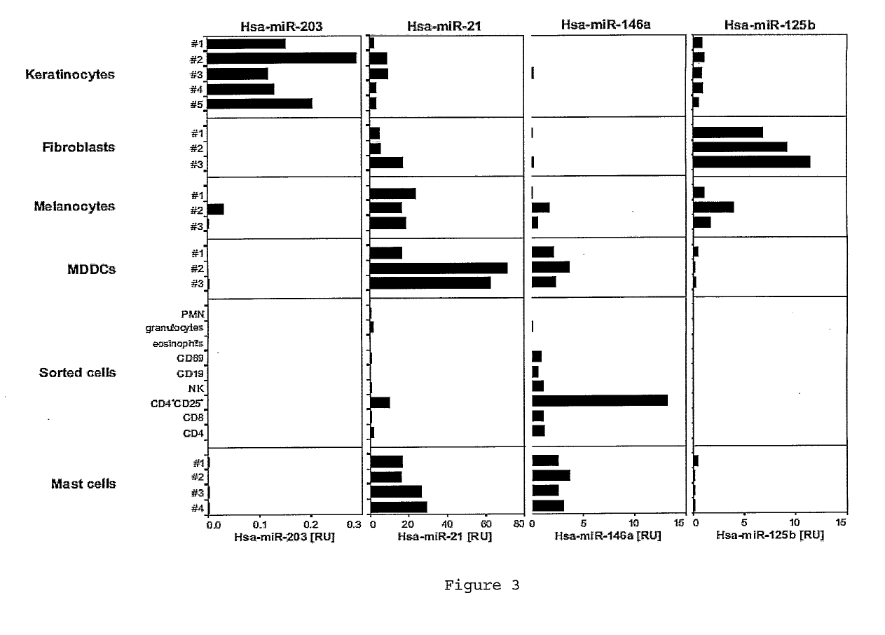

FIG. 3 shows the expression of miR-203, miR-146a, miR-21 and miR-125b in the cellular constituents of the skin. The expressions of the functionally active, mature forms of miR-203, miR-146a, miR-21 and miR-125b were analyzed in the cellular constituents of the skin including primary adult keratinocytes, dermal fibroblasts, melanocytes, monocyte-derived dendritic cells (MDDCs), polymorphonuclear leukocytes (PMN), granulocytes, eosinophils, CD69⁺ cells, CD19⁺ cells, CD56⁺ cells, CD4⁺CD25^{high} cells, CD8⁺ cells, CD4⁺ cells and mast cells using quantitative real-time PCR. Data are expressed in relative units compared to U48 RNA.

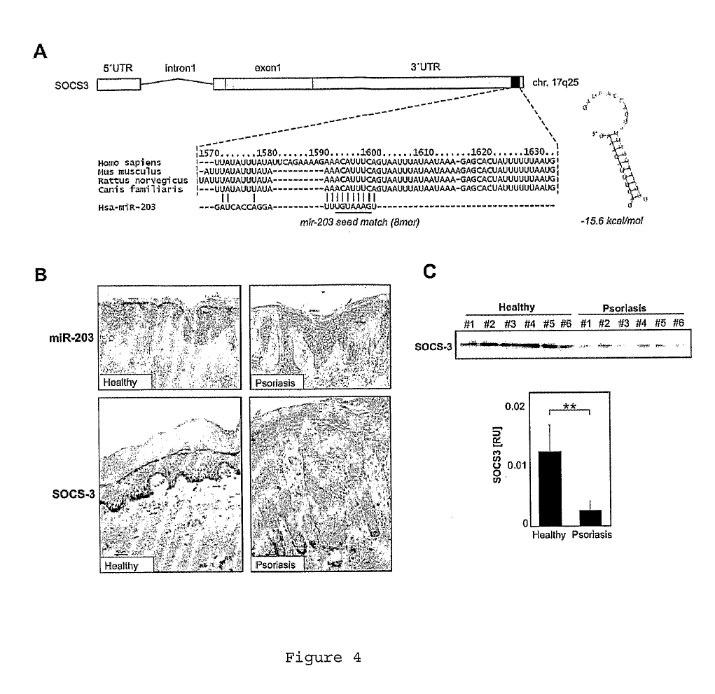

FIG. 4 shows that SOCS3 may be a molecular target of mir-203 post-transcriptional repression. (A) SOCS3 is an evolutionarily conserved target of miR-203. The putative targets site of miR-203 is highly conserved among species. The 8mer seed sequence in the 3-UTR of SOCS3 gene corresponding to miR-203 binding site is underlined. (B) In situ hybridization with LNA-oligonucleotide probes specific to miR-203 in skin sections from healthy skin and psoriatic lesional skin. Data are representative of 6 healthy and 6 psoriatic individuals. Note strong staining in the suprabasal layers of the epidermis in psoriatic skin. The expression of SOCS3 in healthy and psoriasis skin samples was detected using immunohistochemistry. Original magnification 200×. (C) The expression of SOCS-3 in skin from healthy individuals or lesional skin of psoriatic patients was analyzed using Western blot analysis. SOCS-3 protein levels are expressed as relative units. Bars represent means of SOCS-3 protein levels in healthy skin (n=6) and psoriatic lesional skin (n=11) ±SEM. **p<0.01.

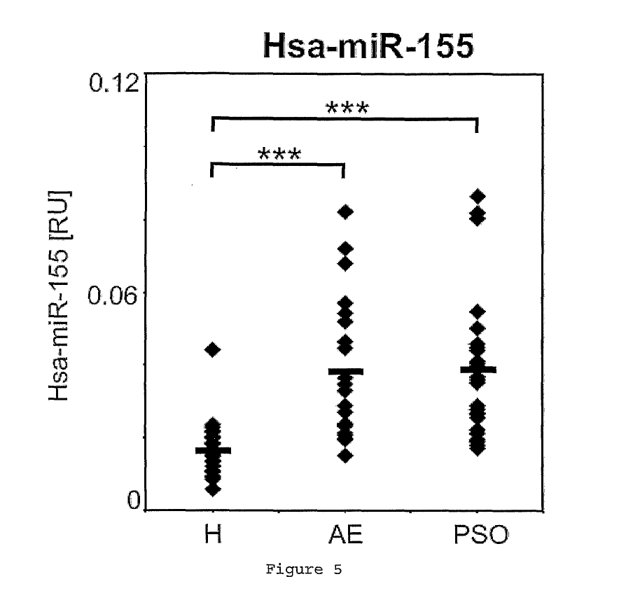

FIG. 5 shows quantitative real-time PCR results which indicate that miR-155 is overexpressed both in atopic eczema (AE) and in psoriasis (PSO) in comparison with healthy skin (H). Data are expressed in relative units compared to U48 RNA. ***p<0.001.

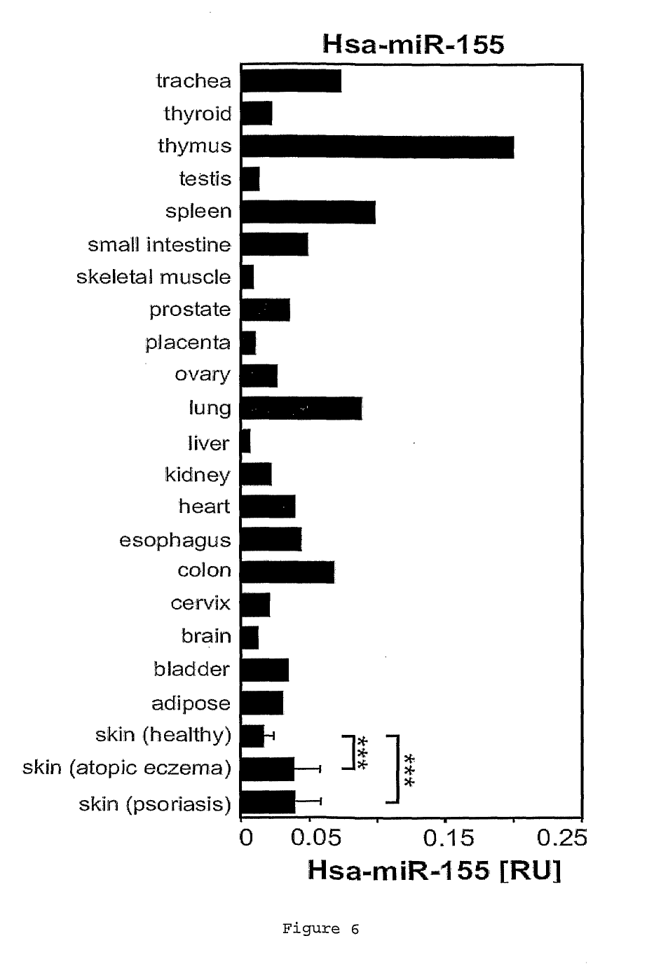

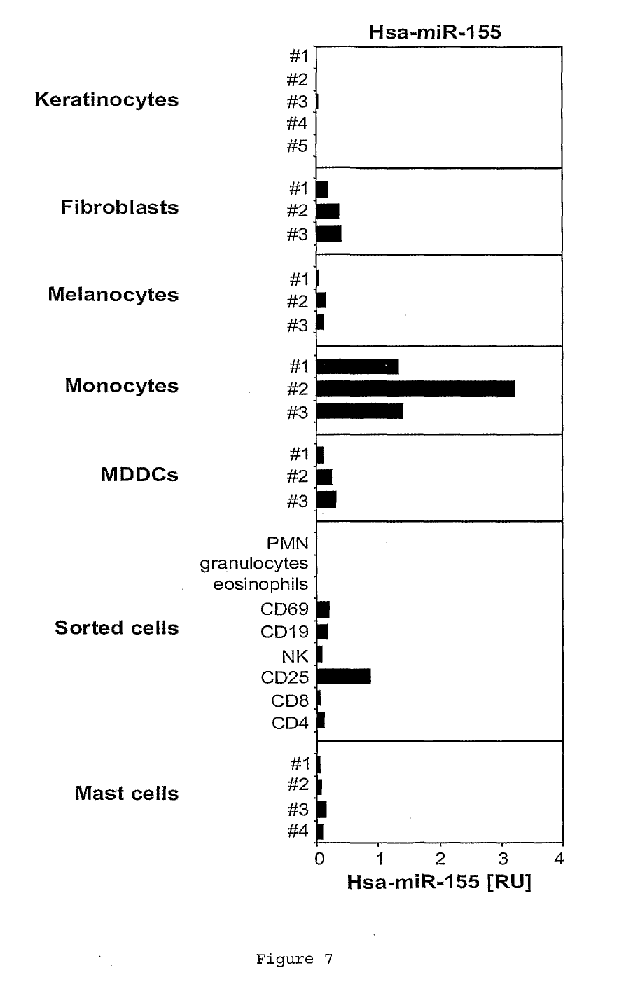

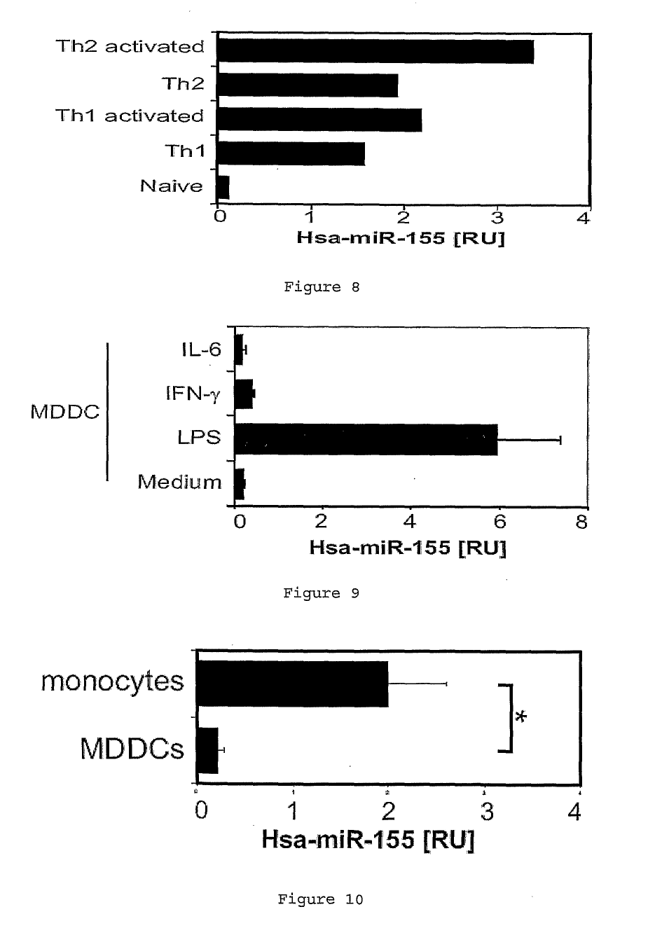

FIG. 6 shows the expression of miR-155 in 20 human organs and tissues.

FIG. 7 shows the expression of miR-155 in different cellular constituents of the skin.

FIG. 8 shows the induction of miR-155 during the differentiation of naïve T cells into mature Th1, Th2 or Treg-like cells.

FIG. 9 shows the expression of miR-155 in dendritic cells after treatment with LPS, IL-6 or IFN-gamma.

FIG. 10 shows the expression of miR-155 during the development of monocytes into dendritic cells.

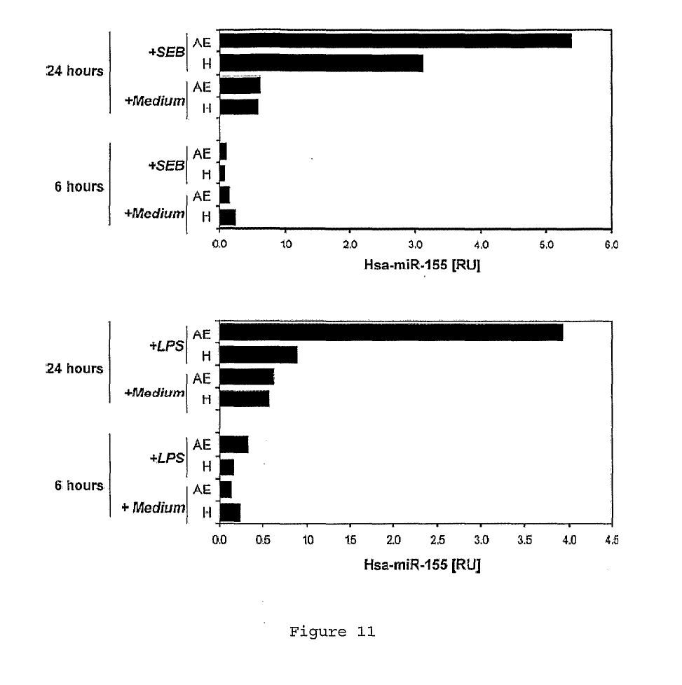

FIG. 11 shows the expression of miR-155 in the PBMCs of atopic eczema patients and healthy individuals following stimulation with SEB or LPS. LPS induces higher MiR-155 expression in the PBMCs of atopic eczema patients relative to healthy individuals.

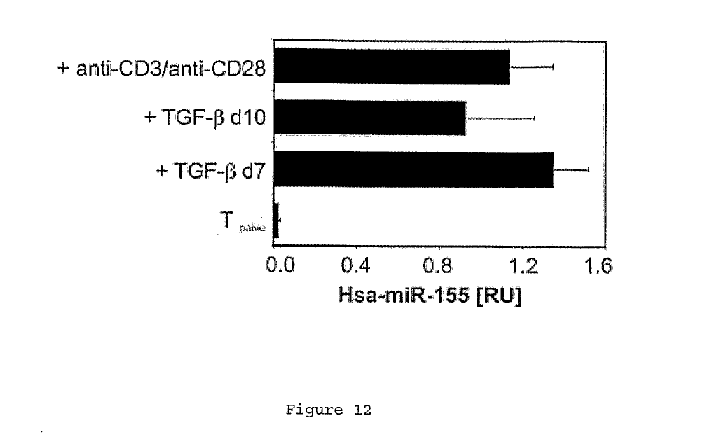

FIG. 12 shows the shows the induction of miR-155 during the following treatment with TGFbeta and anti-CD3/anti-CD28. TGFbeta treated naïve T cells are a model for regulatory T cells (Treg cells) and anti-CD3/anti-CD28 treated naïve T cells represent activated T cells.

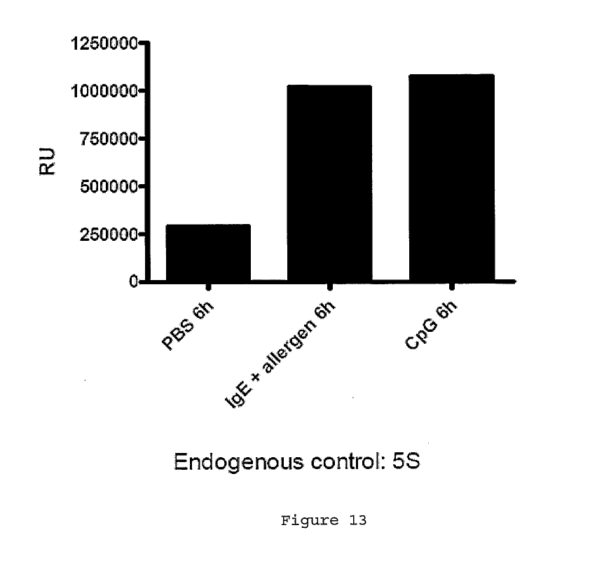

FIG. 13 shows the overexpression of miR-155 in bone marrow-derived mast cells after IgE-crosslinking and CpG exposure.

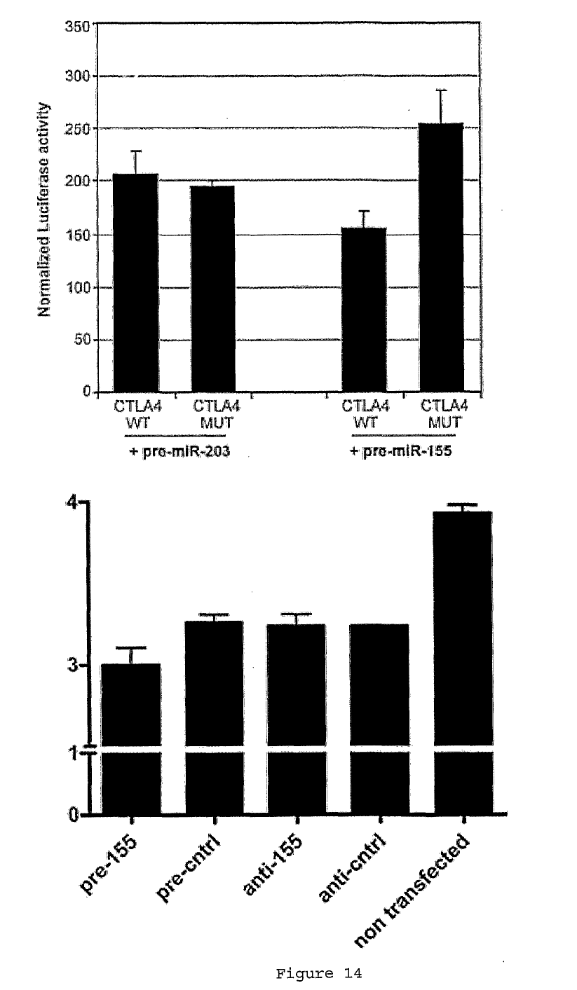

FIG. 14 shows the effect of the cotransfection of specific precursors of miR-155 with CTLA4 3′UTR-luciferase reporter constructs on luciferase activity (top panel) and the effect of miR-155 precursors on CTLA4 expression (bottom panel).

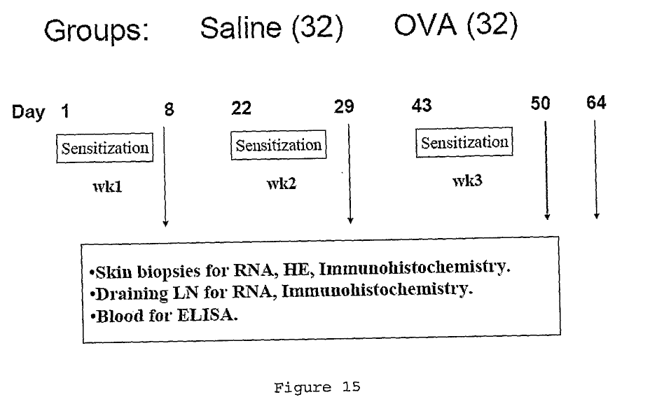

FIG. 15 shows allergen exposure protocol for the murine atopic dermatitis/eczema model. Exposure to ovalbumin (OVA) represents a model for cutaneous allergen exposure.

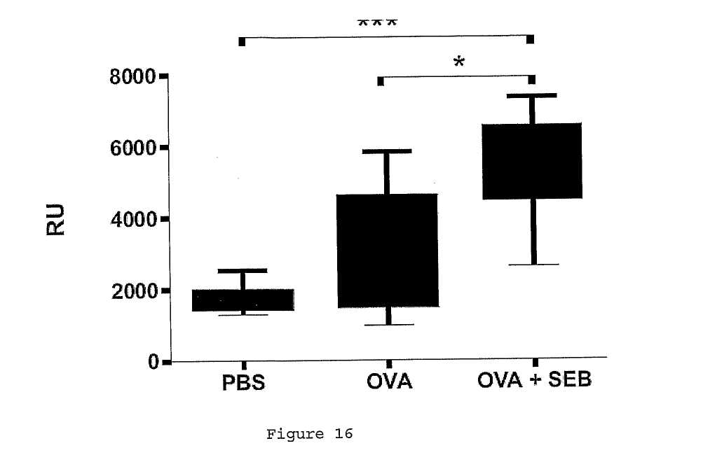

FIG. 16 shows the expression of miR-155 in a mouse model of atopic dermatitis/eczema. Exposure to ovalbumin (OVA) represents a model for cutaneous allergen exposure, while staphylococcal enterotoxin B (SEB) represents a model for cutaneous exposure to superantigen-producing strains of Staphylococcus aureus.

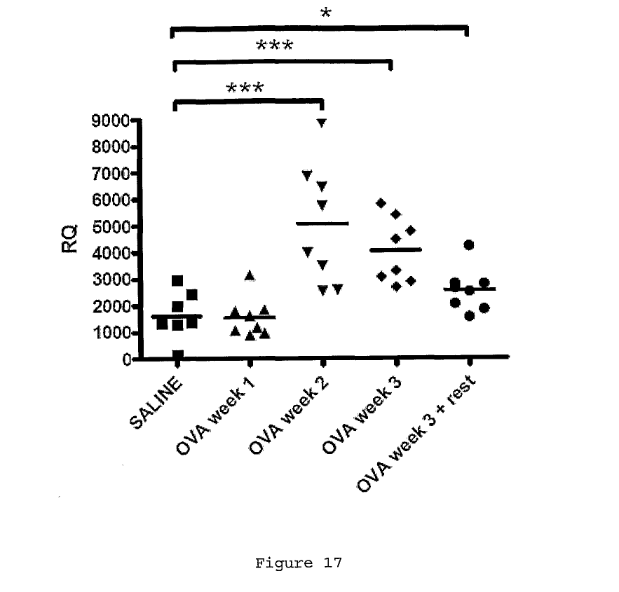

FIG. 17 shows the kinetics of miR-155 expression after allergen exposure.

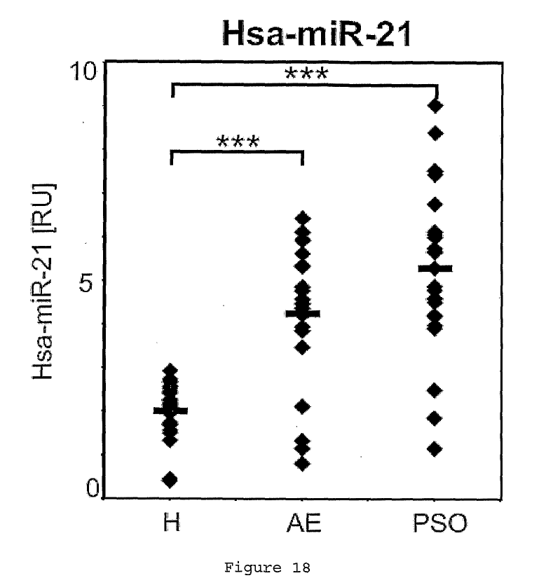

FIG. 18 shows quantitative real-time PCR results which indicate that miR-21 is overexpressed both in atopic eczema (AE) and in psoriasis (PSO) in comparison with healthy skin (H). Data are expressed in relative units compared to U48 RNA. ***p<0.001.

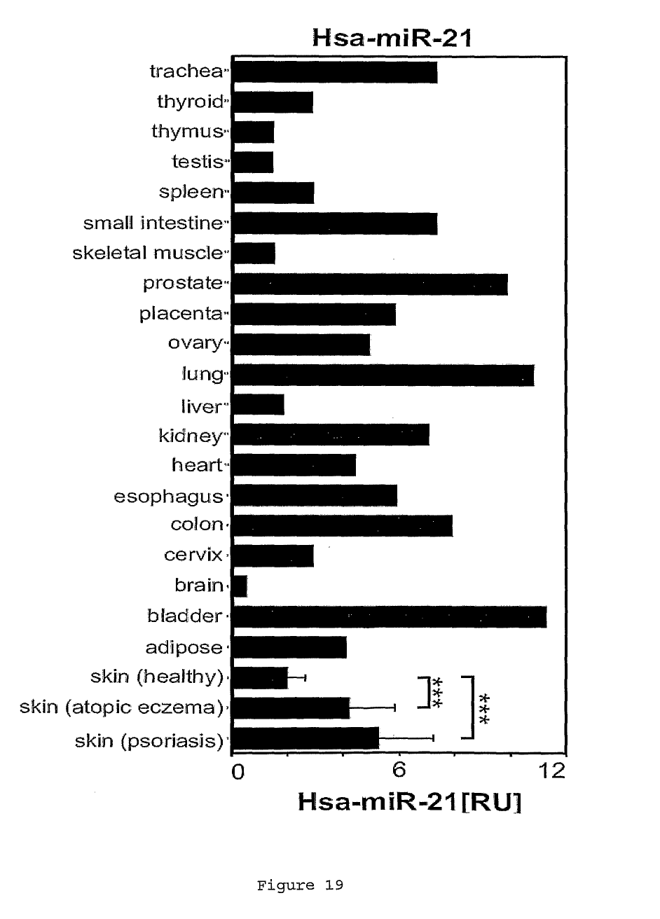

FIG. 19 shows the expression of miR-21 in 20 human organs and tissues.

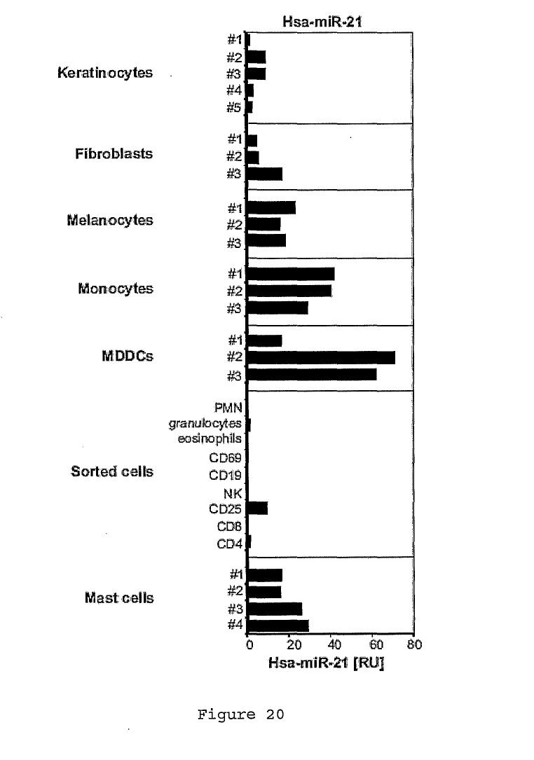

FIG. 20 shows the expression of miR-21 in different cellular constituents of the skin.

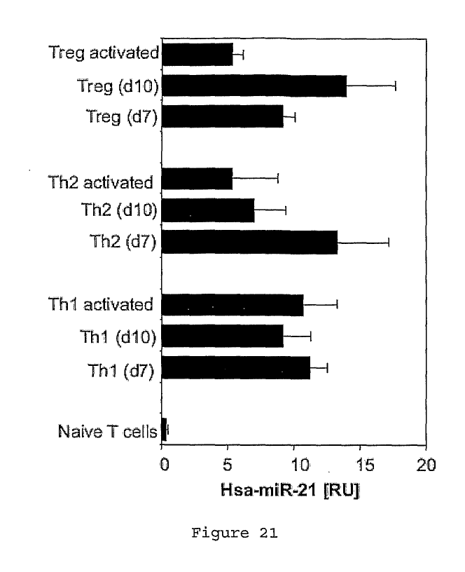

FIG. 21 shows the induction of miR-21 during the differentiation of naïve T cells into mature Th1, Th2 or Treg-like cells.

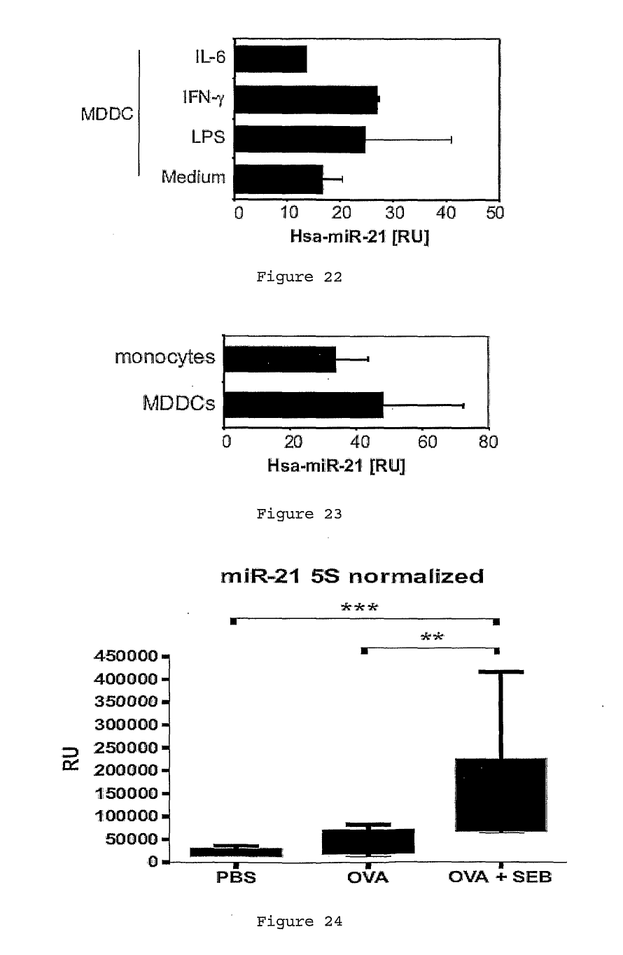

FIG. 22 shows the expression of miR-21 in monocytes-derived dendritic cells (MDDCs) after treatment with LPS, IL-6 or IFN-gamma.

FIG. 23 shows the expression of miR-21 during the development of monocytes into dendritic cells.

FIG. 24 shows the expression of miR-21 in a mouse model of atopic dermatitis/eczema. Exposure to ovalbumin (OVA) represents a model for cutaneous allergen exposure, while staphylococcal enterotoxin B (SEB) represents a model for cutaneous exposure to superantigen-producing strains of Staphylococcus aureus.

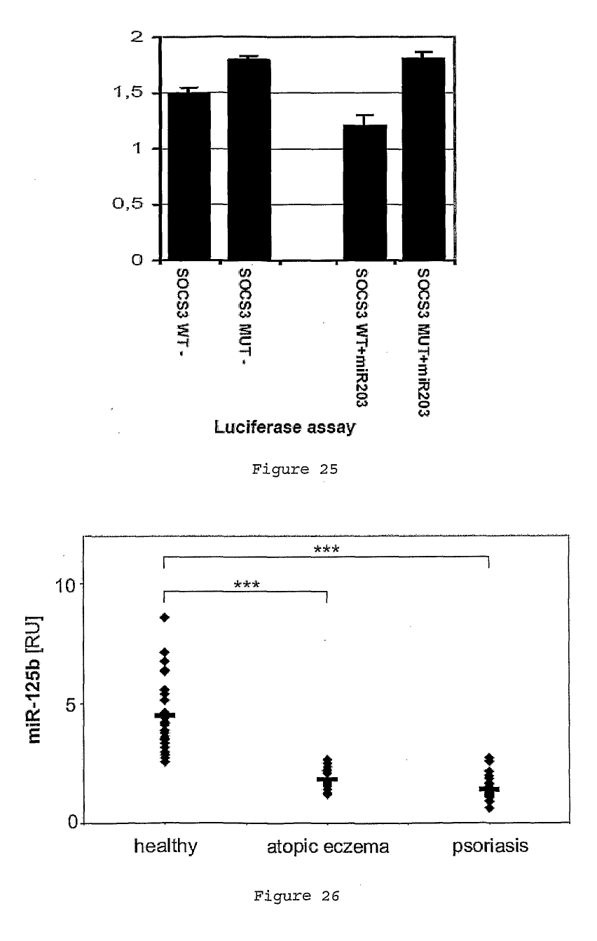

FIG. 25 shows the effect of miR-203 on the expression of a luciferase reporter coupled to the SOCS-3 3′UTR or SOCS-3 3′UTR mutated to disrupt the predicted miR-203 binding site.

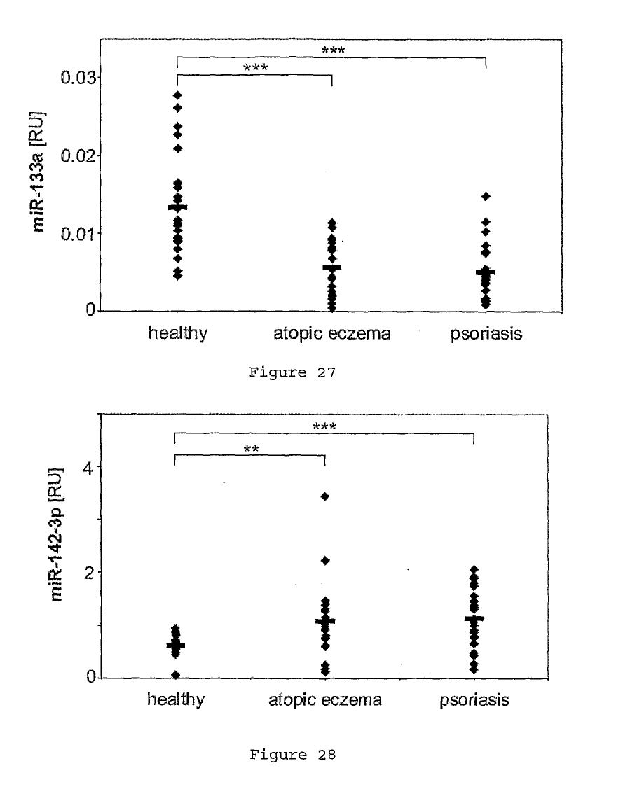

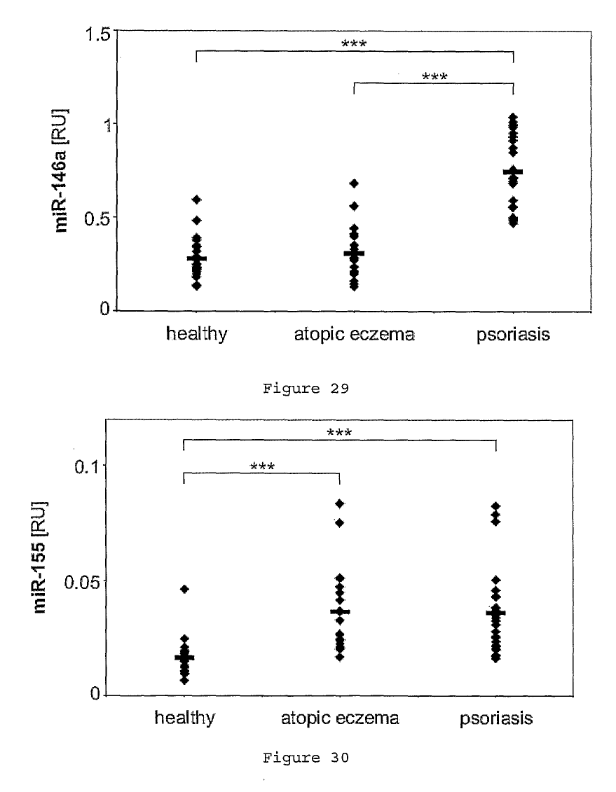

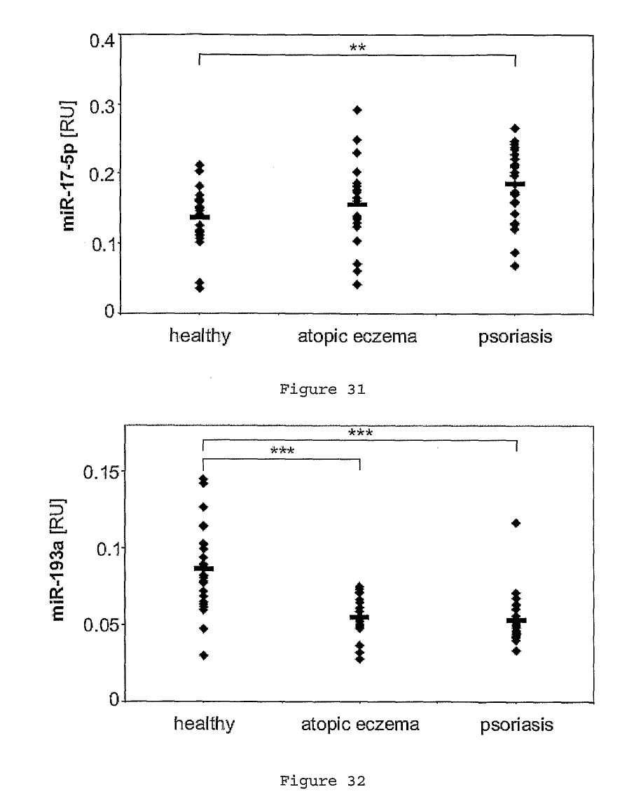

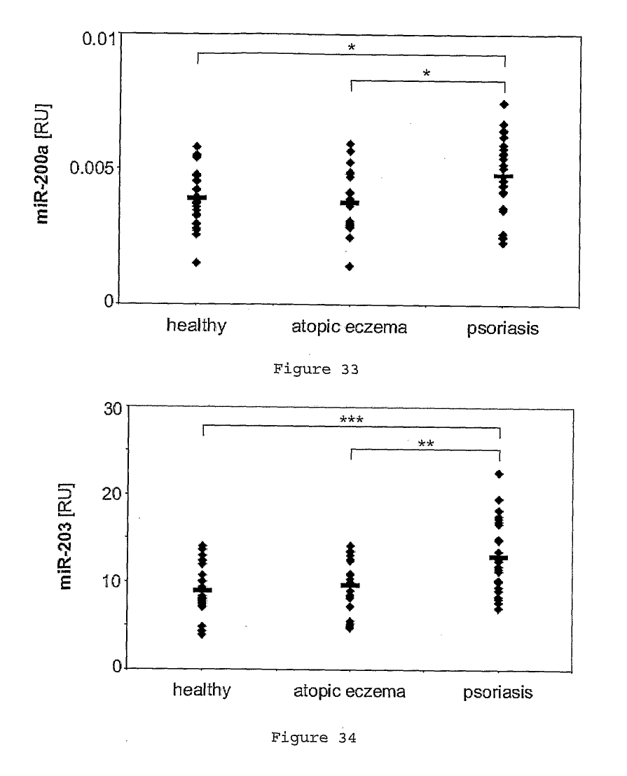

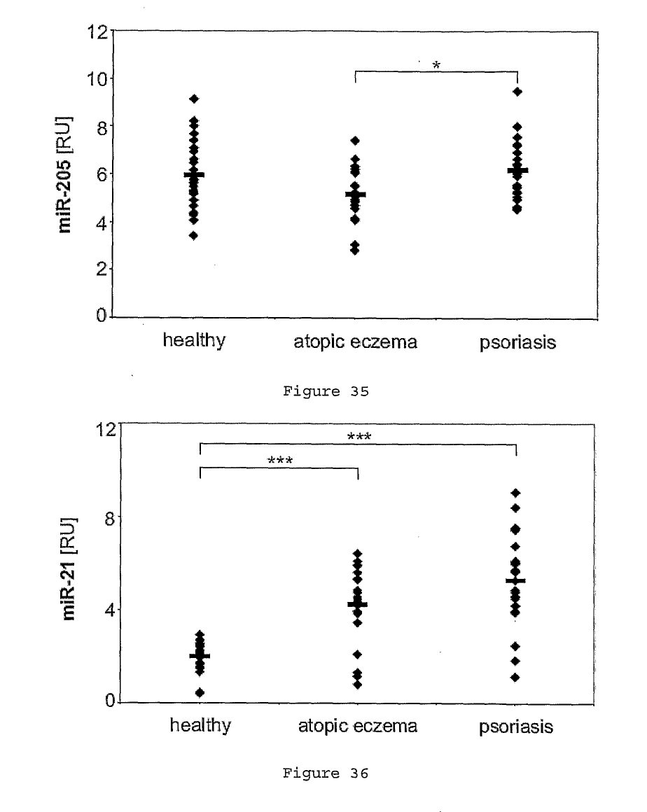

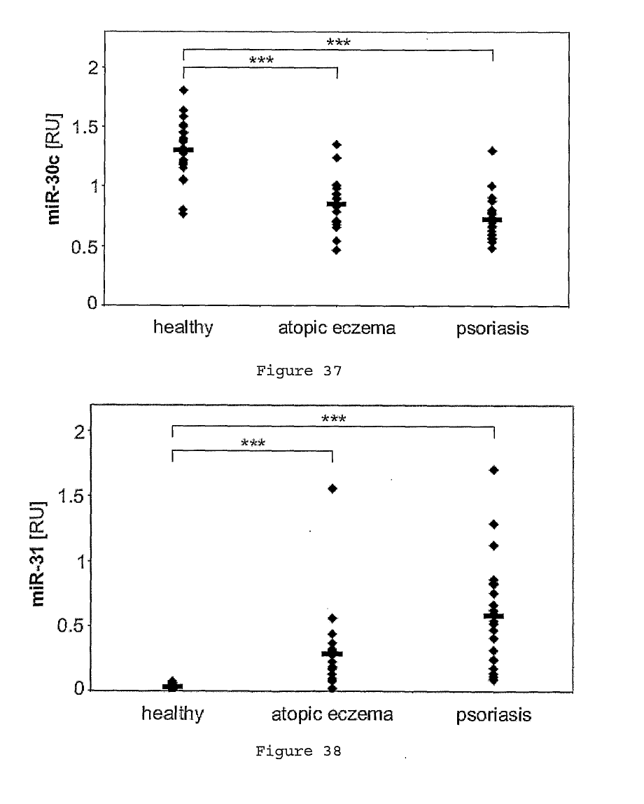

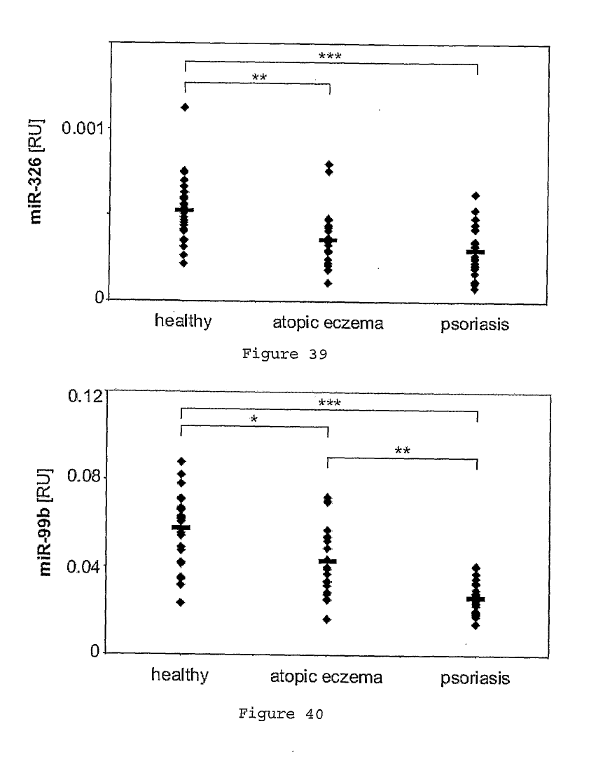

FIGS. 26 to 40 show the results of quantitative real-time PCR using the Taqman™ microRNA assay on 27 healthy, 20 atopic eczema and 25 psoriasis samples showing expression of miR-125b, miR-133a, miR-142-3p, miR-146a, miR-155, miR-17-5p, miR-193, miR-200a, miR-203, miR-205, miR-21, miR-30c, miR-31, miR-326 and miR-99b, respectively. Data are expressed in relative units compared to U48 RNA. ***p<0.001.

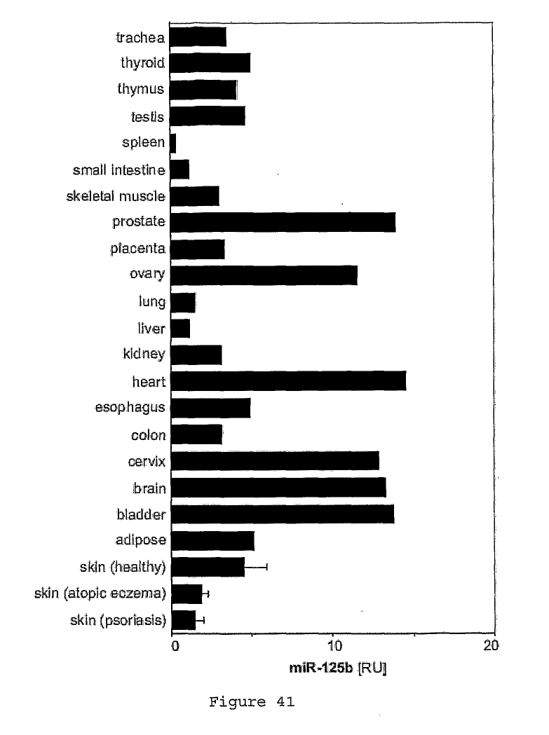

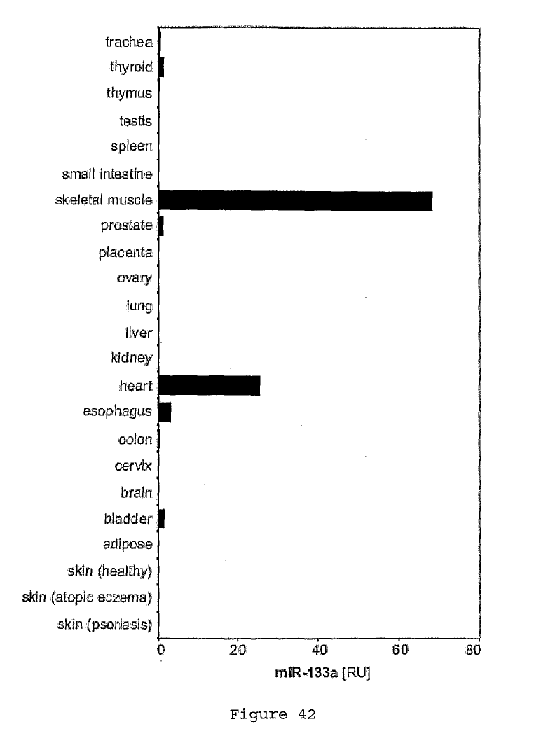

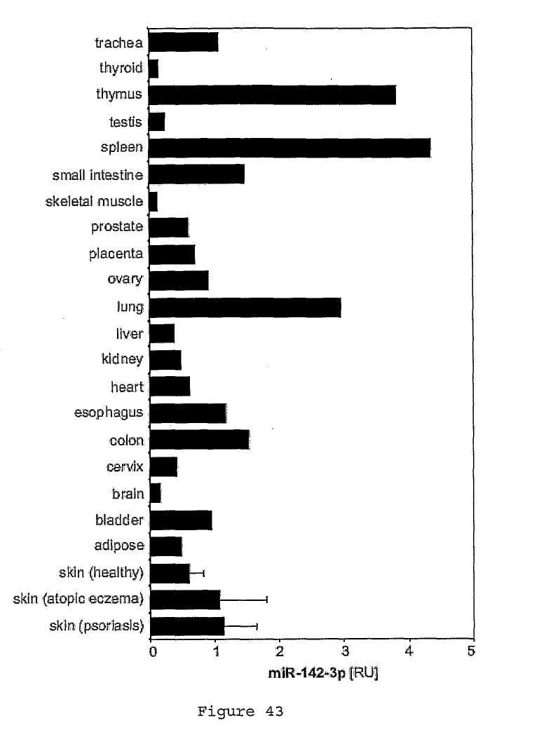

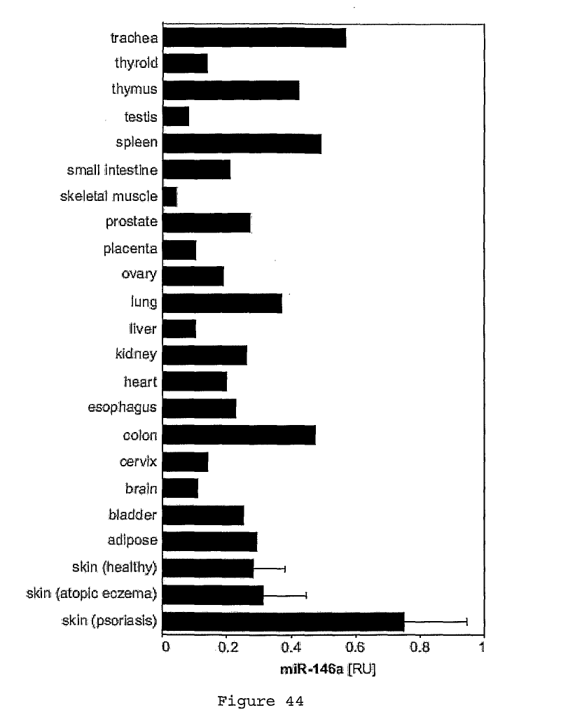

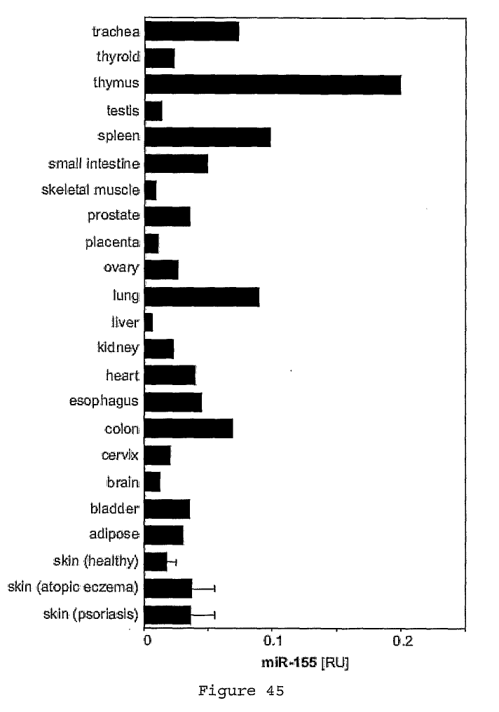

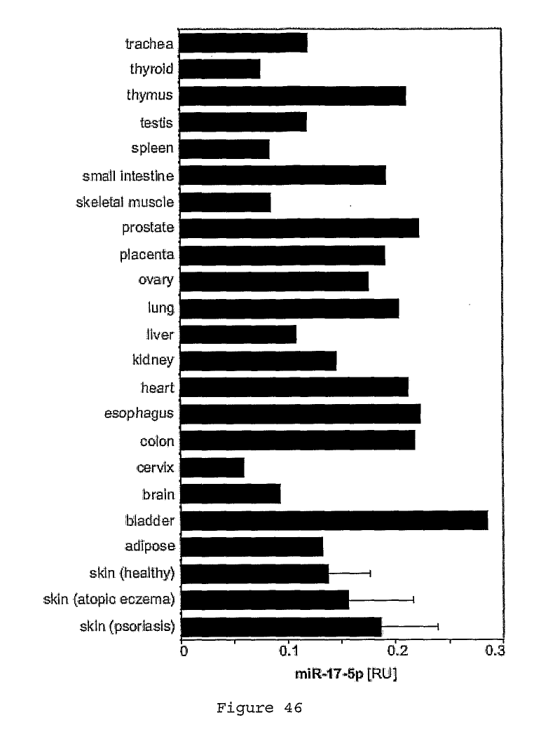

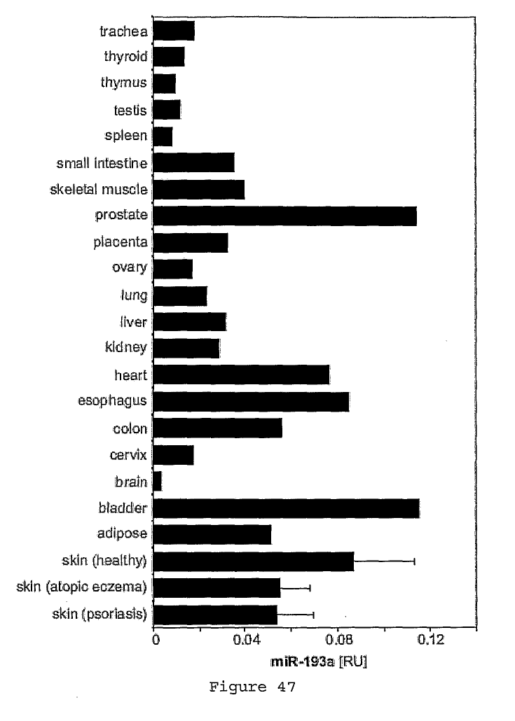

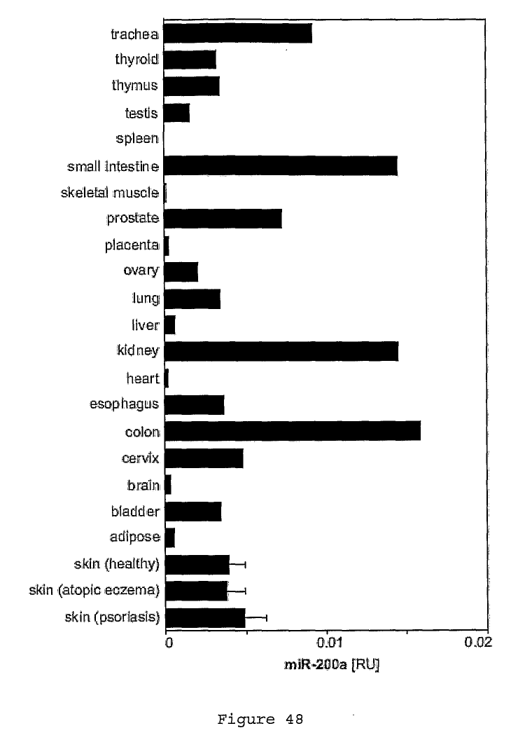

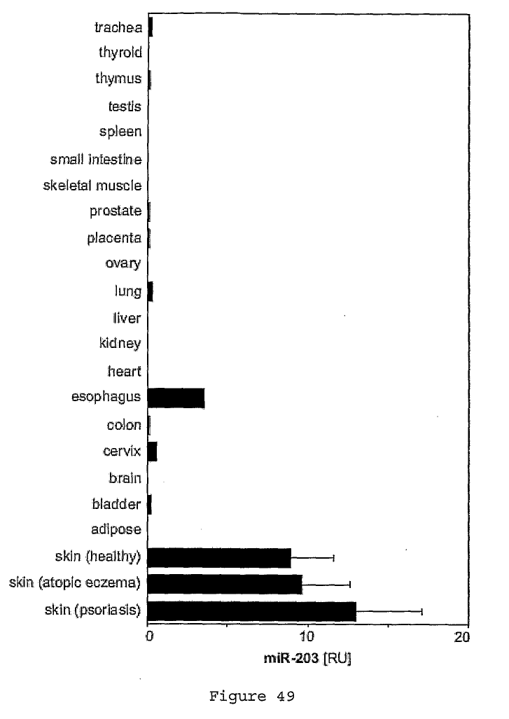

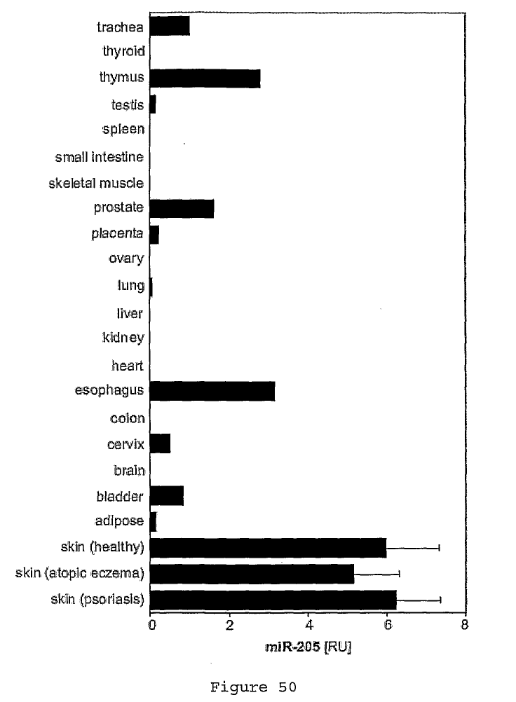

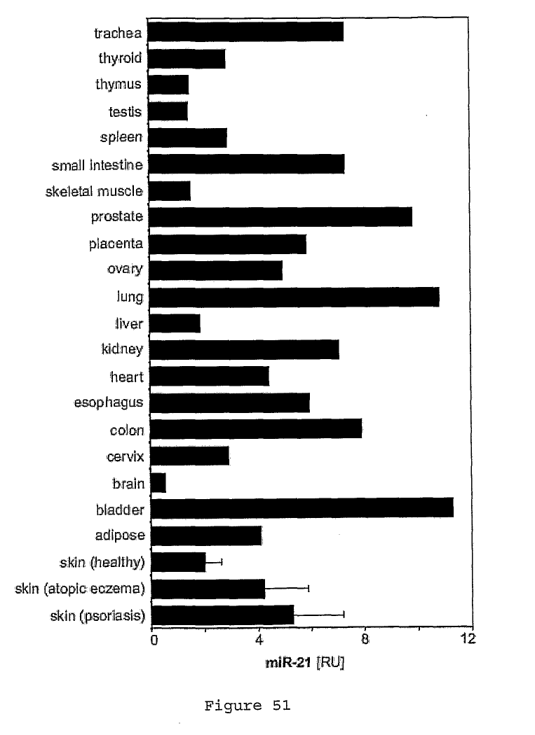

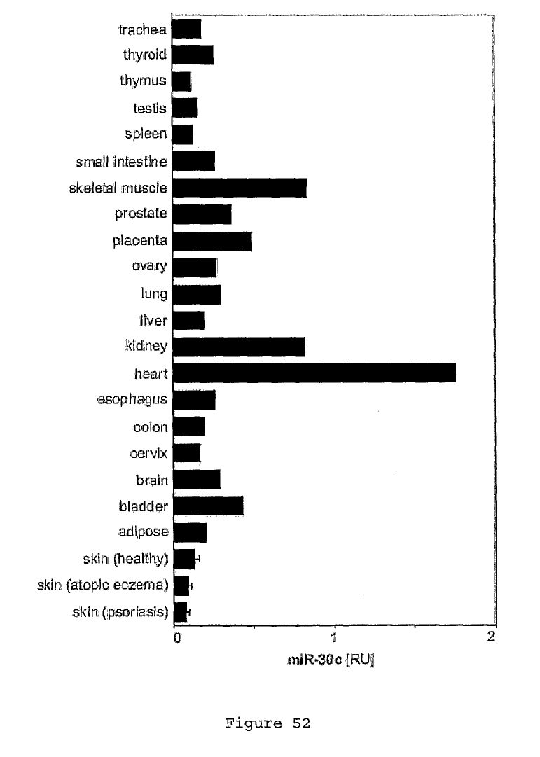

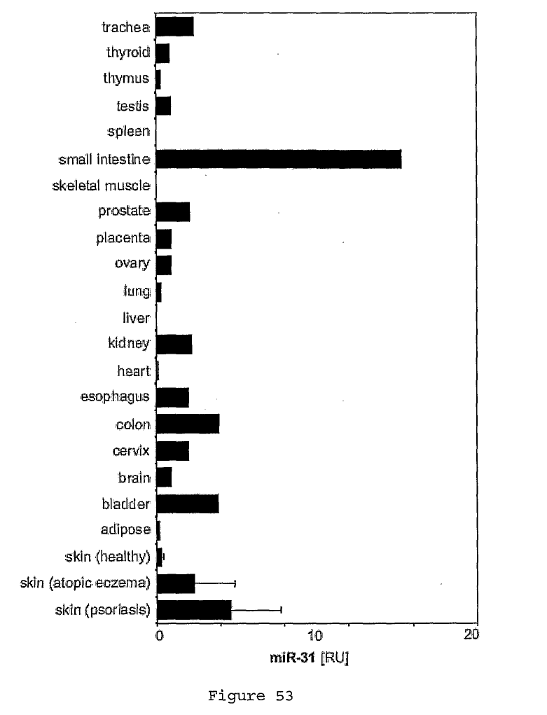

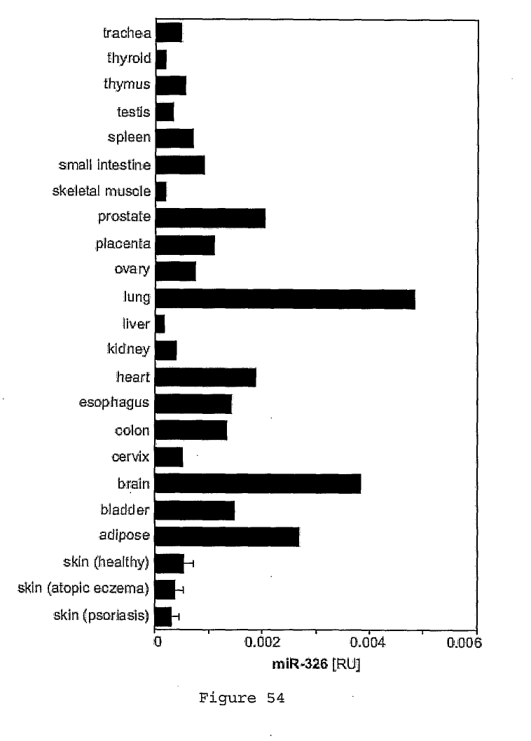

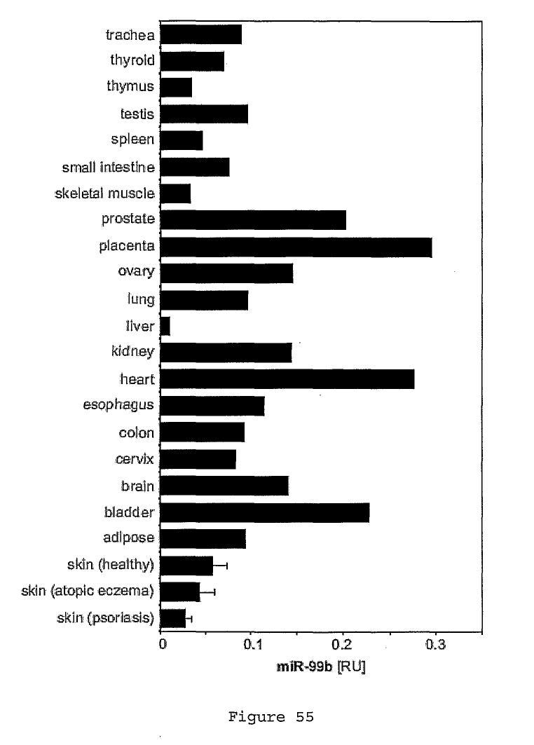

FIGS. 41 to 55 show the results of quantitative real-time PCR using the Taqman™ microRNA assay on 20 different healthy tissue/organs (each from a pool of 3 individuals), 27 healthy, 20 atopic eczema and 25 psoriasis samples. FIGS. 41 to 55 show expression of miR-125b, miR-133a, miR-142-3p, miR-146a, miR-155, miR-17-5p, miR-193, miR-200a, miR-203, miR-205, miR-21, miR-30c, miR-31, miR-326 and miR-99b, respectively, in each tissue/organ sample. Data are expressed in relative units compared to U48 RNA. ***p<0.001.

Table 1 shows miRNAs differentially expressed between healthy skin and psoriasis (left) and healthy skin and atopic eczema (right) according to the SAM algorithm with the cut-off filter set at greater than 1.7-fold down or up-regulation. miRNAs that are over-expressed in both psoriasis and in atopic eczema are highlighted in light grey, miRNAs that are down-regulated in both diseases are highlighted in dark grey.

Table 2 shows the miRBase accession numbers and sequences of the differentially expressed miRNAs.

Table 3 shows miRNAs which were differentially expressed in atopic eczema relative to healthy tissue as identified by significance analysis of microarray results.

Table 4 shows results from further patients setting out miRNAs showing significant increases in expression in atopic eczema lesions relative to healthy tissue as identified by significance analysis of microarray results.

Table 5 shows results from further patients setting out miRNAs showing significant decreases in expression in atopic eczema lesions relative to healthy tissue as identified by significance analysis of microarray results.

Table 6 shows miRNAs which were differentially expressed in psoriasis relative to healthy tissue as identified by significance analysis of microarray results.

Table 7 shows results from further patients setting out miRNAs showing significant increases in expression in psoriasis lesions relative to healthy tissue as identified by significance analysis of microarray results.

Table 8 shows results from further patients setting out miRNAs showing significant decreases in expression in psoriasis lesions relative to healthy tissue as identified by significance analysis of microarray results.

Table 9 shows miRNAs which were differentially expressed in either psoriasis or atopic eczema relative to healthy tissue as identified by significance analysis of microarray results.

Table 10 shows putative targets of miR-155 identified using the targetScan 3.0 algorithm.

### Experiments

### 1. Methods

### 1.1 Cells

Primary human keratinocytes, dermal fibroblasts and melanocytes were isolated from healthy skin using standard protocols and cultured as described (21, 22). Monocytes were isolated from PBMCs from healthy blood donors (Karolinska University Hospital Blood Bank, Stockholm, Sweden) using MACS separation. Immature MDDCs were generated by culturing separated monocytes in the presence of GM-CSF (550 IU/ml), and IL-4 (800 IU/ml) (Biosource International, Camarillo, Calif., USA) for 6 days. CD4+, CD8+, CD4+CD25high, CD56+NK, CD19+, and CD69+ positive cells were isolated from PBMCs from healthy blood donors by FACS sorting using a Becton Dickinson (BD) FACSAria cell sorting system and BD FACSDiva software v 4.1.2. Granulocytes and eosinophils were FACS sorted from whole blood following RBC lysis with ACK lysis buffer.

### 1.2 Patients

Both patients and healthy controls were of Caucasian origin, between 18-65 years old. Patients had not received systemic immunosuppressive treatment or PUVA/solarium/UV, for at least 1 month, and topical therapy for at least 2 weeks before skin biopsy. Four-millimeter punch biopsies were taken, after informed consent, from lesional skin of patients with moderate or severe chronic plaque psoriasis (n=25), lesional skin of patients with moderate to severe chronic atopic eczema (n=20), and from uninflamed, unirritated skin of healthy individuals (n=26). The study was approved by the local ethics committee, and conducted according to the Declaration of Helsinki's principles.

### 1.3 Microarray Experiments

Total RNA from lesional skin of psoriasis patients (n=3) and atopic eczema patients (n=3) and skin of healthy individuals (n=4) was isolated by Trizol (Invitrogen). Two μg of total RNA from each sample were labeled using the miRCURY™ Hy3™/Hy5™ labelling kit and hybridized on the miRCURY™ LNA Array (v.8.0) (Exiqon, Vedbaek, Denmark).

### 1.4 Statistical and Bioinformatics Analysis of Microarray Data

Signal intensities were normalized using the global Lowess regression algorithm. Flagged spots corresponding to absent or low-quality signals were removed from the analysis before global median normalization. For subsequent analysis, we used the log 2 of the background subtracted, normalized median spot intensities of ratios from the two channels (Hy3/Hy5). To find consistently differentially expressed genes, the data were subjected to SAM analysis as described (Tusher, Tibshirani et al. 2001). For visualization of differentially expressed miRNAs, a heat map was generated using TreeView (currently available on-line at http://jtreeview.sourceforge.net).

To identify significance, One-way Analysis of Variance (ANOVA), the Kruskal-Wallis test (Non-parametric ANOVA), Dunn's Multiple Comparisons Test and Bonferroni Multiple Comparisons Test were also employed.

### 1.5 Quantitative Real-Time PCR

Total RNA of skin biopsies and cells was extracted using TRIzol (Invitrogen, Carlsbad, Calif., USA). RNA from 20 different normal human organs was obtained from Ambion (FirstChoice® Human Total RNA Survey Panel). Quantification of miRNAs by TaqMan® Real-Time PCR was carried out as described by the manufacturer (Applied Biosystems, Foster City, Calif.). Briefly, 10 ng of template RNA was reverse transcribed using the TaqMan® MicroRNA Reverse Transcription Kit and miRNA-specific stem-loop primers (Applied Biosystems). 1.5 μl RT product was introduced into the 20 μl PCR reactions which were incubated in 384-well plates on the ABI 7900HT thermocycler (Applied Biosystems) at 95° C. for 10 min, followed by 40 cycles of 95° C. for 15 s and 60° C. for 1 min. Target gene expression was normalized between different samples based on the values of U48 RNA expression.

### 1.6 In Situ Hybridization

In situ transcriptional levels of miR-203 were determined on frozen sections (10 μm) of skin biopsy specimens from six psoriasis patients and six healthy individuals according to the manufacturer's instructions (Exiqon). Sections were hybridized o/n with digoxygenin-labeled miRCURY LNA probes (Exiqon) and incubated with anti-digoxygenin antibody conjugated with alkaline phosphatase for 1 h. Sections were visualized by using BM purple substrate together with 2 mM Levamisole. The color reaction was performed o/n. We followed the protocol recommended by the manufacturer (Exiqon). The stained sections were reviewed with a Zeiss microscope.

### 1.7 Murine Model of Atopic Eczema

The murine model of atopic eczema was generated as previously described (Savinko, Lauerma et al. 2005). Briefly, mice were topically exposed to SEB, OVA, a combination of OVA and SEB (OVA/SEB), or PBS for different time intervals. Skin specimens were obtained from the patched areas, RNA was isolated and the expression of microRNAs was determined by microRNA-specific real-time PCR:

### 2 Results

2.1 A Characteristic miRNA Signature Identified in Psoriasis Skin

At present, the expression and function of miRNAs in human skin is largely unknown. To determine whether miRNAs are involved in the pathogenesis of psoriasis, we performed a comprehensive analysis of all human miRNAs registered in mirBase 8.0 (342 known human miRNAs) in skin lesions of patients with psoriasis (n=3) and compared it to healthy human skin (n=4) or to lesional skin from patients with a nonpsoriatic chronic inflammatory skin disease, atopic eczema (n=3). Analysis of the microarray data showed that miRNAs are expressed in a non-random manner in psoriasis, healthy, or atopic eczema skin (FIG. 1). Using the Significance Analysis of Microarrays (SAM) algorithm (Tusher, Tibshirani et al. 2001), we identified 29 genes that were consistently differentially expressed between psoriasis and healthy skin (Table 1). Among the genes identified in the SAM analysis there were (I) miRNAs specifically up-regulated in psoriasis (e.g. miR-203), (II) miRNAs with increased expression in both psoriasis and atopic eczema (e.g. miR-21), (III) miRNAs specifically down-regulated in psoriasis (e.g. miR-99b), and (IV) miRNAs uniformly down-regulated in both skin diseases (e.g. miR-122a), as compared to healthy skin.

To confirm the results obtained by microarray profiling, we performed quantitative real-time PCR analysis of miR-203, miR-146a, miR-21 and miR-125b expression on RNA samples obtained from lesional skin of patients with psoriasis (n=25), healthy skin (n=26) or atopic eczema lesions (n=20). For this, we used primers designed to amplify specifically the mature, biologically active form of these miRNAs (FIG. 1). In accordance with the microarray data, quantitative real-time PCR results showed significantly (p<0.001) increased miR-203 levels in psoriasis skin when compared with healthy skin. No significant up-regulation of miR-203 was observed in atopic eczema skin lesions compared with healthy skin (FIG. 1). Similarly, miR-146a was significantly over-expressed in psoriatic lesional skin (p<0.001) but not in atopic eczema lesions when compared with healthy skin (FIG. 1). The psoriasis-specific overexpression of miR-203 and miR-146 provides indication that they play specific roles in the pathogenesis of psoriasis and not only a general role in skin inflammation. In contrast to miR-203 and miR-146a, miR-21 was significantly up-regulated both in psoriasis (p<0.001) and atopic eczema (p<0.001) as compared with healthy skin.

miR-125b showed the opposite expression pattern to miR-21: the level of this miRNA significantly (p<0.001 for both) decreased both in psoriasis and atopic eczema. Taken together, psoriasis is characterized by a distinct miRNA expression profile in comparison with healthy skin or with atopic eczema. Of note, one of the identified psoriasis-specific miRNAs, miR-146a was recently shown to regulate the expression of proteins involved in TNF-α-signaling pathway (Taganov, Boldin et al. 2006), which is known to play a central role in psoriatic skin inflammation (Lowes, Bowcock et al. 2007). However, virtually nothing is known about the function of miR-203.

Further quantitative real-time PCR analysis was performed on larger samples (27 healthy, 20 atopic eczema and 25 psoriasis). The results are shown in FIGS. 26 to 40.

The expression of miR-125b, miR-133a, miR-142-3p, miR-155, miR-17-5p, miR-193, miR-21, miR-30c, miR-31 and miR-326 were shown to be significantly associated with both psoriasis and atopic eczema, relative to healthy patients. miR-99b was found to be significantly associated with both psoriasis and atopic eczema relative to healthy patients and with psoriasis relative to atopic eczema patients. miR-146a, miR-200a and miR-203 were found to be significantly associated with psoriasis relative to both healthy and atopic eczema patients. miR-17-5p was found to be significantly associated with psoriasis relative to healthy individuals. miR-205 was found to be significantly associated with psoriasis relative to atopic eczema patients.

2.2 miR-203, miR-21, miR-146a and miR-125b Show Distinct Expression Patterns in Human Organs and Cells Types

At present, the expression pattern of the miRNAs we identified in skin is largely unknown in different organs and tissues. To obtain further insights into the function of the psoriasis-associated miRNAs, we systematically analyzed the expression of miR-203, miR-146a, miR-21 and miR-125b in skin and a panel of 20 additional human organs obtained from healthy individuals (FIG. 2). Quantitative real-time PCR analysis showed that miR-203, a miRNA specifically up-regulated in psoriasis, was expressed more than 100-fold higher in skin compared with most other organs. In addition to skin, miR-203 was only expressed at lower levels in organs that also contain squamous epithelium, esophagus and cervix. These findings provide indication for a specific function for this miRNA in the formation or function of squamous epithelia. In contrast to miR-203, the mature forms of miR-146a, miR-21 and miR-125b were detected in all studied organs, however, their expression showed distinct patterns. miR-146a was highly expressed in organs containing significant number of leukocytes such as the thymus and the spleen, and showed low expression in healthy skin, providing indication that infiltrating cells express miR-146a in the skin (FIG. 2). MiR-21 showed highest expression in the lung, trachea, colon, prostate and bladder (FIG. 2), while miR-125b, a miRNA downregulated in both psoriasis and atopic eczema, was expressed mostly in organs that contain cells of ectodermal origin, including cervix, brain and bladder.

In psoriasis, there is evidence for the pathogenic relevance of several different cell types that normally occur in skin: keratinocytes (Sano, Chan et al. 2005), fibroblasts (Dimon-Gadal, Gerbaud et al. 2000), monocyte-derived immunocytes (Nestle, Conrad et al. 2005; Lowes, Bowcock et al. 2007), T cells (Nickoloff and Wrone-Smith 1999), and mast cells (Fischer, Harvima et al. 2006). Therefore it is likely that this disease is the outcome of aberrantly activated mechanisms that do not necessarily depend on one single cell type but involve a variety of different cell populations (Stratis, Pasparakis et al. 2006; Lowes, Bowcock et al. 2007). To identify the cell types expressing the identified psoriasis-associated miRNAs in the skin, we systematically analyzed the expression of miR-203, miR-146a, miR-21 and miR-125b in a panel of cells present in healthy and/or inflamed skin including both resident cells (keratinocytes, dermal fibroblasts and melanocytes) and leukocyte/immune cell subsets (CD4+, CD8+ and CD4CD25^{high} T cell subsets, NK cells, granulocytes, B cells, dendritic cells and mast cells). In accordance with their expression profiles in different organs, the identified miRNAs showed a distinctive expression pattern in the studied cell types. miR-203, which was specifically expressed in skin among 21 different human organs, showed a keratinocyte-specific expression being virtually absent in all other cell types analyzed (FIG. 3). This observation provides indication for a role for this miRNA in keratinocyte functions in healthy skin as well as in psoriasis. By contrast, miR-146a was absent from keratinocytes and dermal fibroblasts, and it was preferentially expressed by immune cells (FIG. 3), in accordance with its high expression in immune organs (FIG. 2) and in the psoriatic inflamed skin. In particular, CD4CD25^{high} regulatory T cells, monocyte-derived dendritic cells (MDDCs) and mast cells expressed miR-146a at a high level. The abundant expression of miR-146a in regulatory T cells provides indication that this miRNA might influence the function of regulatory T cells in psoriatic skin. miR-21, a microRNA upregulated in both psoriasis and atopic eczema, was expressed both by structural and inflammatory cells (FIG. 2). The expression pattern of miR-125b was complementary to that of mir-146a: it was expressed at a very low level in inflammatory cells in comparison to structural cells: fibroblasts, keratinocytes and melanocytes (FIG. 2).

The expression patterns of miR-125b, miR-133a, miR-142-3p, miR-146a, miR-155, miR-17-5p, miR-193, miR-200a, miR-203, miR-205, miR-21, miR-30c, miR-31, miR-326 and miR-99b were analysed in 20 different healthy tissue/organs in a sample of 27 healthy, 20 atopic eczema and 25 psoriasis patients. The results are shown in FIGS. 41 to 55.

It is widely accepted that psoriasis is not a disease caused by one cell type but a consequence of impaired cross talk between the immune system and the structural cells of the skin (Lowes, Bowcock et al. 2007). Investigating the cellular distribution of miRNAs deregulated in psoriasis we found that these master switches of gene expression are expressed in cells with key roles in the pathogeneses of psoriasis. Deregulation of both keratinocyte- and leukocyte-specific miRNAs in psoriasis indicates that altered miRNA-mediated gene regulation may contribute to the disturbed cross talk between keratinocytes and immune cells. One of the most important mediators in leukocyte-keratinocyte interactions in psoriasis is tumor necrosis factor alpha (TNF-α) as evidenced by the effectiveness of TNF-α inhibitors in the treatment of psoriasis (Lowes, Bowcock et al. 2007). Interestingly, a recent study showed that miR-146a, one of the psoriasis-specific miRNAs, inhibits the expression of IRAK-1 and TRAF-6 proteins both of which are regulators of the TNF-α signaling pathway (Taganov, Boldin et al. 2006). Hence, it is conceivable that miR-146a is involved in the pathogenesis of psoriasis via the modulation of TNF-α signaling in the skin. In contrast to miR-146, nothing is known about the function of the keratinocyte-specific miRNA, miR-203.

2.3 miR-203 and Keratinocyte Dysfunction in Psoriasis Through the Regulation of Suppressor of Cytokine Signalling 3 (SOCS-3)

Epidermal keratinocytes are active participants in the formation of psoriasis plaques (Lowes, Bowcock et al. 2007). Psoriatic keratinocytes show abnormal differentiation and proliferation, have aberrant cell signaling and produce mediators that contribute to the recruitment and activation of immune cells (Lowes, Bowcock et al. 2007). The specific expression of miR-203 in skin and in keratinocytes (FIGS. 2 and 3) as well as its specific up-regulation in psoriasis (FIG. 1) indicates that this miRNA plays a role in the regulation of keratinocyte functions. Thus, we next focused on characterizing miR-203 in more detail.

Since miRNAs exert their effect by regulating the expression of protein-coding genes, their function can be interpreted as the sum of the function of the genes they regulate (Bartel 2004). To understand the functions of miR-203 we used a two-step sequential approach: (I) using algorithms based on a systematic analysis of the structural requirements for target site function in vivo, we predicted genes that can be regulated by this miRNA; (II) we investigated the biological functions of the predicted target genes. To explore whether the presence of miR-203 binding sites in the 3′ untranslated region (UTR) of mRNAs correlates with gene function, we determined if putative miR-203 targets contain significantly more or fewer genes from any given biological process than expected given the gene ontology (GO) category's frequency in the 3′UTR database. Out of the several thousand GO categories, the top significant (p<0.01) target categories were dominated by processes related to signal transduction, cell cycle, morphogenesis and cell growth indicating a role for miR-203 in the regulation of these biological processes in the skin. Of note, these GO categories show significant overlap with the biological processes that are strongly perturbed in the psoriatic skin lesions (Zhou, Krueger et al. 2003).

Among the target genes of miR-203, we focused on the suppressor of cytokine signaling-3 (SOCS-3), an evolutionarily conserved high-score target of miR-203 with a 10-nucleotide complementarity to the mature, biologically active form of miR-203 in human, mouse, rat and dog (FIG. 4A). SOCS-3 is part of a negative feedback loop in cytokine signaling inhibiting the activation of STAT3, a transcription factor whose activation in keratinocytes is essential for the development of psoriatic plaques (Sano, Chan et al. 2005; Lowes, Bowcock et al. 2007). However, SOCS-3 has not been associated with psoriasis.

Next, we analyzed the expression pattern of miR-203 and SOCS-3 in lesional skin of psoriasis patients and in healthy skin (FIG. 4B). In situ hybridization using LNA-modified nucleotide probes revealed that miR-203 is expressed in the suprabasal layers of the epidermis in normal skin, confirming its keratinocyte-specific expression observed in vitro (FIG. 4B). In comparison to healthy skin, the expression of miR-203 was increased in psoriatic lesional skin in all epidermal layers (FIG. 4B), consistent with the real-time PCR results. Analysis of SOCS-3 protein expression by immunohistochemistry showed a complementary pattern with the miR-203 expression (FIG. 4B). SOCS-3 was strongly expressed by the basal layer of keratinocytes in healthy skin, while it was suppressed in the epidermis of psoriasis lesions. Down-regulation of SOCS-3 expression in psoriatic lesional skin was further confirmed by Western blot analysis, demonstrating a significant (p<0.01) decrease in SOCS-3 protein levels in psoriatic plaques as compared to healthy skin (FIG. 4C). Since the decrease of SOCS3 protein in psoriatic skin could be due to decreased transcriptional activity of the gene, we analyzed SOCS3 mRNA levels in psoriatic and healthy skin. However, quantitative real time PCR analysis showed no significant difference in SOCS-3 mRNA expression between psoriatic and healthy skin, providing indication that the down-regulation of SOCS-3 in psoriasis occurs at the posttranscriptional level.

SOCS-3 deficiency leads to sustained activation of STAT-3 in response to IL-6 (Croker, Krebs et al. 2003), a cytokine present in the psoriasis lesions (Lowes, Bowcock et al. 2007). This provides indication that the suppression of SOCS-3 by miR-203 in psoriatic lesions would in turn lead to constant activation of STAT3. Indeed, the psoriatic hyperplastic epidermis shows increased STAT3 activation and constitutively active STAT3 in keratinocytes leads to the spontaneous development of psoriasis in transgenic mice (Sano, Chan et al. 2005). Thus, the up-regulation of miR-203 may have important implications for psoriasis pathogenesis by preventing the up-regulation of SOCS-3 in response to cytokines.

Suppression of SOCS-3 in psoriatic keratinocytes may lead to sustained activation of the STATS pathway, leading to the infiltration of leukocytes and the development of psoriatic plaques.

In addition to the modulation of inflammatory responses, SOCS-3 has also been implicated in the regulation of keratinocyte proliferation and differentiation. It has been shown that overexpression of SOCS-3 in keratinocytes leads to final differentiation and inhibits serum-stimulated proliferation (Goren, Linke et al. 2006). miRNA-mediated suppression of SOCS-3 expression in keratinocytes may therefore not only modulate cytokine signaling but also contribute to keratinocyte hyperproliferation and alteration in keratinocyte differentiation in psoriatic plaques.

Significant repression of the reporter was observed when the SOCS-3 3″UTR was coupled to a luciferase reporter and cotransfected with the miR-203 precursor into HeLa cells. This repression was alleviated when the predicted binding site was mutated (FIG. 25).

2.4 miR-155 is Expressed in T-Cells and Dendritic Cells and Induced in a Mouse Model of Atopic Eczema

The expression of the functionally active, mature form of miR-155 was analyzed using quantitative real-time PCR in the skin of 26 healthy individuals, and lesional skin samples of 20 patients with atopic eczema and 25 patients with psoriasis. The results for individual patients and mean are shown in FIG. 5. Data are expressed in relative units compared to U48 RNA (***p<0.001). miR-155 was found to be overexpressed both in atopic eczema and in psoriasis in comparison with healthy skin.

In order to compare the expression of miR-155 in skin to that of other organs, we analyzed its expression in 21 different tissue and organ samples. miR-155 showed the highest expression in lymphoid organs such as thymus, spleen, lung and colon, while its expression in health skin was relatively low (FIG. 6), providing indication that miR-155 is mainly expressed in immune cells. Among the cellular constituents of the skin, miR-155 was found to be expressed by regulatory T cells, dendritic cells (MDDCs), fibroblasts, monocytes and mast cells (FIG. 7). These results show that miR-155 is expressed in multiple immune cell types.

A significant, ˜10-fold induction during the differentiation of naïve T cells into mature Th1, Th2 or Treg-like cells (FIG. 8) and tightly regulated expression of miR-155 was observed during T-cell differentiation (FIG. 12). A dramatic increase in miR-155 in dendritic cells was observed after LPS treatment, which induces the maturation of dendritic cells. However, IL-6 or IFN-gamma had no effect (FIG. 9). We also observed a significant ˜10-fold down-regulation in miR-155 during the development of monocytes into dendritic cells (FIG. 10).

Expression of MiR-155 in the PBMCs of atopic eczema patients was increased relative to healthy individuals following stimulation with SEB or LPS (FIG. 11). The difference became detectable 24 hours after stimulation with SEB or LPS. LPS, IL-4 had no effect on miR-155 expression in monocytes but LPS, and IgE cross-linking induced miR-155 in human mast cells (CBMCs) (FIG. 13).

Patients with atopic eczema have repeated cutaneous exposure to both environmental allergens and superantigen-producing strains of Staphylococcus aureus. Topical exposure of mouse skin to SEB, OVA, or a combination of OVA and SEB of mice results in skin inflammation resembling atopic eczema and thus represents an established animal model for investigating this disease. A suitable protocol to generate the model is shown in FIG. 15.

We have analyzed miR-155 expression in the skin of animals sensitized to SEB or OVA using real-time PCR after exposure to SEB or OVA/SEB. Both SEB and OVA/SEB induced miR-155 expression (FIG. 16), indicating that (1) allergens and superantigens may contribute to the overexpression of miR-155 in human atopic eczema lesions (2) this mouse model is suitable for further in vivo studies of miR-155 functions in atopic eczema. The kinetics of miR-155 expression following allergen exposure are shown in FIG. 17.

Putative targets of miR-155 were identified using targetScan 3.0 algorithm (table 10). The occurrence of GO terms associated with miR-155 targets were analyzed using the Gene ontology Tree Machine and the Gene Set Analysis Toolkit (http://bioinfo.vanderbilt.edu/webgestalt; Vanderbilt University). Based on the predicted targets of miR-155, this miRNA was found to play a role in inter alia in the positive regulation of interleukin-1 biosynthesis [Gene Ontology annotation GO:0045362] and lymphocyte differentiation [GO:0030098]. Gene Ontology Consortium annotations are described in Nature Genet. (2000) 25: 25-29.

Preliminary analysis using TargetScan and PicTar software (Sonkoly, E., et al., PLoS ONE, 2007. 2 (7): p. e610) has identified Cytotoxic T-Lymphocyte Antigen 4 (CTLA-4), an inhibitory molecule present mainly on CD4⁺ T cells, as one of the evolutionary conserved putative targets of miR-155. Cotransfection of specific precursors of miR-155 with CTLA4 3′UTR-luciferase reporter constructs was found to result in decreased luciferase activity (FIG. 14). Furthermore, transfection of T cells with miR-155 precursors result in decreased CTLA4 expression (FIG. 14). These results show that CTLA4 is a target of miR-155.

The highly regulated expression of miR-155 during T cell and dendritic cell differentiation together with the significant enrichment of target genes associated with differentiation provides indication that miR-155 regulates T cell and dendritic cell differentiation. The interactions between T cells and dendritic cells are crucial in the development of skin inflammation in both psoriasis and atopic eczema. Therefore, miR-155 represents a potential therapeutic target in the treatment of these chronic inflammatory skin diseases.

2.5 miR-21 is Expressed by Both Structural and Immune Cells and Induced in a Mouse Model of Atopic Eczema

miR-21 was found to be overexpressed both in atopic eczema and in psoriasis in comparison with healthy skin. The expression of the functionally active, mature forms of miR-21 was analyzed using quantitative real-time PCR in the skin of 26 healthy individuals, and lesional skin samples of 20 patients with atopic eczema and 25 patients with psoriasis. The results for individual patients and mean are shown in FIG. 18. Data are expressed in relative units compared to U48 RNA (***p<0.001).

Among 20 human organs and tissues, miR-21 was found to be expressed ubiquitously, with highest levels in bladder, lung, prostate and trachea (FIG. 19). Among the cellular constituents of the skin, miR-21 was expressed by structural cells (keratinocytes, fibroblasts, melanocytes), monocytes and dendritic cells, CD4+CD25high regulatory T cells and mast cells (FIG. 20).

A significant, induction during the differentiation of naïve T cells into mature Th1, Th2 or Treg-like cells was observed (FIG. 21) but no significant change was observed in miR-21 levels in dendritic cells after LPS treatment, which induces the maturation of dendritic cells. IL-6 or IFN-gamma also did not affect miR-21 expression (FIG. 22). A slight up-regulation of miRNA was observed during the development of monocytes into dendritic cells (FIG. 23) and miR-21 was significantly upregulated in a mouse model of atopic eczema (FIG. 24).

Putative targets of miR-21 were identified using targetScan 3.0 algorithm. The occurrence of GO terms associated with miR-21 targets were analyzed using the Gene ontology Tree Machine and the Gene Set Analysis Toolkit (http://bioinfo.vanderbilt.edu/webgestalt; Vanderbilt University). Based on the predicted targets of miR-21, this miRNA plays a role inter alia in the regulation of transcription factor activity [GO:0051090], nervous system development [GO:0007399], negative regulation of signal transduction [GO:0009968], negative regulation of G-protein coupled receptor protein signalling pathway [GO:0045744], T cell receptor signaling pathway [GO:0050852], regulation of cell migration [GO:0030334], apoptosis [GO:0006915], regulation of growth [GO:0040008], cell cycle [GO:0007049], cell proliferation [GO:0008283], positive regulation of alpha-beta T cell proliferation [GO:0046641], JAK-STAT cascade [GO:0007259] and signal transduction [GO:0007165]. Predicted target genes of miR-21 are involved in the regulation of growth (fibroblast growth factor), differentiation (Jagged-1, Kerato-epithelin), apoptosis (SAMD7) and cell migration (IL-1a, Ephrin B2)

These results provide indication that miR-21 is involved in the regulation of growth, differentiation, apoptosis and cell migration. These cellular processes are altered in chronic inflammatory skin diseases. Therefore, miR-21 represents a potential therapeutic target in the treatment of these chronic inflammatory skin diseases.

Taken together, miRNA expression patterns distinguish psoriasis from healthy skin and from another chronic inflammatory skin disease, atopic eczema. Results reported here reveal a new layer of regulatory mechanisms in the pathogenesis of chronic inflammatory skin diseases. Our data provide indication that miR-203 plays a specific role in the pathogenesis of psoriasis by regulating inflammation-, proliferation- and morphogenesis-associated processes in the skin. Interestingly, miRNAs have been recently implicated in the morphogenesis of murine skin (Yi, O'Carroll et al. 2006). Since miRNAs are master switches that ultimately affect complex cellular processes and functions through the regulation of several proteins, miRNA-based therapies may be more effective than drugs targeting single proteins. The disease-specific miRNAs identified in our study represent potential therapeutic targets in the treatment of chronic skin inflammation.

### REFERENCES

Ambros, V. (2004). Nature 431 (7006): 350-5.
Bartel, D. P. (2004) Cell 116 (2): 281-97.
Babak et al., RNA, 10:1813-1819 (2004)
Carlen, L. M. et al. (2005). J Invest Dermatol 124 (1): 63-9.
Castoldi, M., S. Schmidt, et al. (2006) Rna 15: 15.
Chen, C. et al. (2005). Nucleic Acids Res 33 (20): e179.
Cheng, A. M. et al. (2005). Nucleic Acids Res 33 (4): 1290-7.
Croker, B. A., D. L. Krebs, et al. (2003). Nat Immunol 4 (6): 540-5.
Czech, M. P. (2006). N Engl J Med 354 (11): 1194-5.
Dimon-Gadal, S. et al. (2000). J Invest Dermatol 114 (5): 984-9.
Fischer, M. et al. (2006). J Clin Invest 116 (10): 2748-56.
Goren, I. et al. (2006). J Invest Dermatol 126 (2): 477-85.
Krutzfeldt, J. et al. (2007). Nucleic Acids Res.
Krutzfeldt, J. et al. (2005). Nature 438 (7068): 685-9.
Kim et al (2006 TRENDS in Genetics 22 .3 165-173
Lebwohl, M. (2003). The Lancet 361 (9364): 1197-1204.
Leung, D. Y. et al. (2004). J Clin Invest 113 (5): 651-7.
Liu et al. Proc. Nat'l Acad. Sci. USA, 101: 9740-9744 (2004)
Lowes, M. A. et al. (2007). Nature 445 (7130): 866-73.
Mattes et al Am. J. Respir. Cell Mol. Biol. 36, 1 Jan. 2007, 8-12
Nestle, F. O., C. Conrad, et al. (2005). J Exp Med 202 (1): 135-43.
Nickoloff, B. J. et al (1999). Am J Pathol 155 (1): 145-58.
Nomura, I. et al. (2003). J Allergy Clin Immunol 112 (6): 1195-202.
Orom, U. A. et al. (2006). Gene 372: 137-41.
Sano, S. et al. (2005). Nat Med 11 (1): 43-9.
Savinko et al. (2005) J Immunol. 2005 Dec. 15; 175 (12):8320-6
Stratis, A. et al. (2006). J Clin Invest 116 (8): 2094-104.
Taganov, K. D. et al. (2006). PNAS USA 103 (33): 12481-6.
Thomson et al., Nature Methods, 1:1-7 (2004)
Tusher, V. G. et al. (2001). Proc Natl Acad Sci USA 98 (9): 5116-21.
Vermeulen, A., B. Robertson, et al. (2007). Rna 13 (5): 723-30.
Yi, R., D. O'Carroll, et al. (2006). Nat Genet. 38 (3): 356-62.
Zhou, X. et al. (2003). Physiol Genomics. 13 (1): 69-78.

TABLE 1

| Psoriasis | | | Atopic eczema | | | |
|---|---|---|---|---|---|---|
| miRNA | Score | fold change | miRNA | Score | fold change | |
| miR-20a | 5.17 | 3.15 | miR-146a | 2.85 | 1.68 | INDUCED |
| miR-146a | 5.06 | 2.93 | let-7i | 2.50 | 2.04 | |
| miR-146b | 4.68 | 3.31 | miR-29a | 2.39 | 1.83 | |
| miR-31 | 3.47 | 4.69 | miR-222 | 2.22 | 1.67 | |
| miR-146a | 3.04 | 3.67 | miR-24 | 2.20 | 1.85 | |
| miR-20a | 2.99 | 2.64 | miR-193a | 2.14 | 2.27 | |
| miR-200a | 2.85 | 2.75 | miR-199a | 2.13 | 1.72 | |
| miR-17-5p | 2.67 | 3.77 | miR-27a | 2.04 | 1.72 | |
| miR-30e-5p | 2.61 | 3.61 | miR-21 | 2.04 | 3.26 | |
| miR-141 | 2.26 | 3.45 | miR-20a | 2.03 | 2.43 | |
| miR-203 | 2.23 | 5.86 | miR-20a | 2.01 | 2.28 | |
| miR-142-3p | 2.22 | 2.55 | miR-146a | 1.84 | 2.75 | |
| miR-21 | 2.21 | 2.51 | miR-17-5p | 1.82 | 2.58 | |
| miR-106a | 2.16 | 2.37 | miR-106b | 1.78 | 1.72 | |
| miR-125b | −5.31 | 0.55 | miR-122a | −2.75 | 0.19 | SUPPRESSED |
| miR-99b | −3.32 | 0.58 | miR-133a-133b | −2.73 | 0.28 | |
| miR-122a | −3.09 | 0.18 | miR-326 | −2.50 | 0.39 | |
| miR-197 | −2.69 | 0.64 | miR-215 | −2.48 | 0.42 | |
| miR-100 | −2.62 | 0.59 | miR-483 | −1.89 | 0.57 | |
| miR-381 | −2.60 | 0.71 | miR-519d | −1.86 | 0.48 | |
| miR-518b | −2.55 | 0.56 | miR-335 | −1.78 | 0.63 | |
| miR-524* | −2.40 | 0.50 | miR-133b | −1.68 | 0.23 | |
| let-7e | −2.25 | 0.60 | miR-515-5p | −1.57 | 0.43 | |
| miR-30c | −1.98 | 0.63 | | | | |
| miR-365 | −1.87 | 0.62 | | | | |
| miR-133b | −1.78 | 0.22 | | | | |
| miR-10a | −1.78 | 0.67 | | | | |
| miR-133a-133b | −1.76 | 0.40 | | | | |
| miR-22 | −1.68 | 0.61 | | | | |
| miR-326 | −1.61 | 0.56 | | | | |
| miR-215 | −1.59 | 0.56 | | | | |

TABLE 2

| | | | |
|---|---|---|---|
| miRNA (hsa-miR) | Accession No | Sequence | SEQ ID NO: |

| | | | |
|---|---|---|---|
| miR-1 | MIMAT0000416 | UGGAAUGUAAAGAAGUAUGUA | 1 |
| miR-9 | MIMAT0000441 | UCUUUGGUUAUCUAGCUGUAUGA | 2 |
| miR-10a | MIMAT0000253 | UACCCUGUAGAUCCGAAUUUGUG | 3 |
| miR-10b | MIMAT0000254 | UACCCUGUAGAACCGAAUUUGU | 4 |
| miR-15a | MIMAT0000068 | UAGCAGCACAUAAUGGUUUGUG | S |
| miR-15b | MIMAT0000417 | UAGCAGCACAUCAUGGUUUACA | 6 |
| miR-16 | MIMAT00000G9 | UAGCAGCACGUAAAUAUUGGCG | 7 |
| miR-17-5p | MIMAT0000070 | CAAAGUGCUUACAGUGCAGGUAG | 8 |
| miR-20a | MIMAT0000075 | UAAAGUGCUUAUAGUGCAGGUAG | 9 |
| miR-21 | MIMAT0000076 | UAGCUUAUCAGACUGAUGUUGA | 10 |
| miR-22 | MIMAT0000077 | AAGCUGCCAGUUGAAGAACUGU | 11 |
| miR-23a | MIMAT0000078 | AUCACAUUGCCAGGGAUUUCC | 12 |
| miR-23b | MIMAT0000418 | AUCACAUUGCCAGGGAUUACC | 13 |
| miR-24 | MIMAT0000079 | GUGCCUACUGAGCUGAUAUCAGU | 14 |
| miR-26a | MIMAT0000082 | UUCAAGUAAUCCAGGAUAGGCU | 15 |
| miR-26b | MIMAT0000083 | UUCAAGUAAUUCAGGAUAGGU | 16 |
| miR-27a | MIMAT0000084 | UUCACAGUGGCUAAGUUCCGC | 17 |
| miR-27b | MIMAT0000419 | UUCACAGUGGCUAAGUUCUGC | 18 |
| miR-29a | MIMAT0000086 | UAGCACCAUCUGAAAUCGGUU | 19 |
| miR-29c | MIMAT0000681 | UAGCACCAUUUGAAAUCGGUUA | 20 |
| miR-30a-5p | MIMAT0000087 | UGUAAACAUCCUCGACUGGAAG | 21 |
| miR-30a-3p | MIMAT0000088 | CUUUCAGUCGGAUGUUUGCAGC | 22 |
| miR-30b | MIMAT0000420 | UGUAAACAUCCUACACUCAGCU | 23 |
| miR-30c | MIMAT0000244 | UGUAAACAUCCUACACUCUCAGC | 24 |
| miR-30d | MIMAT0000245 | UGUAAACAUCCCCGACUGGAAG | 25 |
| miR-30e-3p | MIMAT0000693 | CUUUCAGUCGGAUGUUUACAGC | 26 |
| miR-30e-5p | MIMAT0000692 | UGUAAACAUCCUUGACUGGA | 27 |
| miR-31 | MIMAT0000089 | GGCAAGAUGCUGGCAUAGCUG | 28 |
| miR-92 | MIMAT0000092 | UAUUGCACUUGUCCCGGCCUGU | 29 |
| miR-93 | MIMAT0000093 | CAAAGUGCUGUUCGUGCAGGUAG | 30 |
| miR-95 | MIMAT0000094 | UUCAACGGGUAUUUAUUGAGCA | 31 |
| miR-99a | MIMAT0000097 | AACCCGUAGAUCCGAUCUUGUG | 32 |
| miR-99b | MIMAT0000689 | CACCCGUAGAACCGACCUUGCG | 33 |
| miR-100 | MIMAT0000098 | AACCCGUAGAUCCGAACUUGUG | 34 |
| miR-101 | MIMAT0000099 | UACAGUACUGUGAUAACUGAAG | 35 |
| miR-106a | MIMAT0000103 | AAAAGUGCUUACAGUGCAGGUAGC | 36 |
| miR-106b | MIMAT0000680 | UAAAGUGCUGACAGUGCAGAU | 37 |
| miR-107 | MIMAT0000104 | AGCAGCAUUGUACAGGGCUAUCA | 38 |
| miR-122a | MIMAT0000421 | UGGAGUGUGACAAUGGUGUUUGU | 39 |
| miR-125a-5p | MIMAT0000443 | UCCCUGAGACCCUUUAACCUGUGA | 40 |
| miR-125a-3p | MIMAT0004602 | ACAGGUGAGGUUCUUGGGAGCC | 41 |
| miR-125b | MIMAT0000423 | UCCCUGAGACCCUAACUUGUGA | 42 |
| miR-126 | MIMAT0000445 | UCGUACCGUGAGUAAUAAUGCG | 43 |
| miR-127-3p | MIMAT0000446 | UCGGAUCCGUCUGAGCUUGGCU | 44 |
| miR-127-5p | MIMAT0004604 | CUGAAGCUCAGAGGGCUCUGAU | 45 |
| miR-130a | MIMAT0000425 | CAGUGCAAUGUUAAAAGGGCAU | 46 |
| miR-130b | MIMAT0000691 | CAGUGCAAUGAUGAAAGGGCAU | 47 |
| miR-132 | MIMAT0000426 | UAACAGUCUACAGCCAUGGUCG | 48 |
| miR-133a | MIMAT0000427 | UUGGUCCCCUUCAACCAGCUGU | 49 |
| miR-133b | MIMAT0000770 | UUGGUCCCCUUCAACCAGCUA | 50 |
| miR-135b | MIMAT0000758 | UAUGGCUUUUCAUUCCUAUGUGA | 51 |
| miR-140-5p | MIMAT0000431 | CAGUGGUUUUACCCUAUGGUAG | 52 |
| miR-40-3p | MIMAT0004597 | UACCACAGGGUAGAACCACGG | 53 |
| miR-141 | MIMAT0000432 | UAACACUGUCUGGUAAAGAUGG | 54 |
| miR-142-3p | MIMAT0000434 | UGUAGUGUUUCCUACUUUAUGGA | 55 |
| miR-142-5p | MIMAT0000433 | CAUAAAGUAGAAAGCACUACU | 56 |
| miR-143 | MIMAT0000435 | UGAGAUGAAGCACUGUAGCUCA | 57 |
| miR-145 | MIMAT0000437 | GUCCAGUUUUCCCAGGAAUCCCU | 58 |
| miR-146a | MIMAT0000449 | UGAGAACUGAAUUCCAUGGGUU | 59 |
| miR-146b | MIMAT0002809 | UGAGAACUGAAUUCCAUAGGCU | 60 |
| miR-148a | MIMAT0000243 | UCAGUGCACUACAGAACUUUGU | 62. |
| miR-148b | MIMAT0000759 | UCAGUGCAUCACAGAACUUUGU | 62 |
| miR-149 | MIMAT0000450 | UCUGGCUCCGUGUCUUCACUCCC | 63 |
| miR-152 | MIMAT0000438 | UCAGUGCAUGACAGAACUUGG | 64 |
| miR-155 | MIMAT0000646 | UUAAUGCUAAUCGUGAUAGGGG | 65 |
| miR-181d | MIMAT0002821 | AACAUUCAUUGUUGUCGGUGGGU | 66 |
| miR-182 | MIMAT0000259 | UUUGGCAAUGGUAGAACUCACACU | 67 |
| miR-186 | MIMAT0000456 | CAAAGAAUUCUCCUUUUGGGCU | 68 |
| miR-187 | MIMAT0000262 | UCGUGUCUUGUGUUGCAGCCGG | 69 |
| miR-191 | MIMAT0000440 | CAACGGAAUCCCAAAAGCAGCUG | 70 |
| miR-193a | MIMAT0000459 | AACUGGCCUACAAAGUCCCAG | 71 |
| miR-193b | MIMAT0002819 | AACUGGCCCUCAAAGUCCCGCU | 72 |
| miR-194 | MIMAT0000460 | UGUAACAGCAACUCCAUGUGGA | 73 |
| miR195 | MIMAT0000461 | UAGCAGCACAGAAAUAUUGGC | 74 |
| miR-196a | MIMAT0000226 | UAGGUAGUUUCAUGUUGUUGGG | 75 |
| miR-196b | MIMAT0001080 | UAGGUAGUUUCCUGUUGUUGGG | 76 |
| miR-197 | MIMAT0000227 | UUCACCACCUUCUCCACCCAGC | 77 |
| miR-199a | MIMAT0000231 | CCCAGUGUUCAGACUACCUGUUC | 78 |
| miR-199b | MIMAT0000263 | CCCAGUGUUUAGACUAUCUGUUC | 79 |
| miR-200a | MIMAT0000682 | UAACACUGUCUGGUAACGAUGU | 80 |
| miR-200b | MIMAT0000318 | UAAUACUGCCUGGUAAUGAUGAC | 81 |
| miR-200c | MIMAT0000617 | UAAUACUGCCGGGUAAUGAUGG | 82 |
| miR-203 | MIMAT0000264 | GUGAAAUGUUUAGGACCACUAG | 83 |
| miR-204 | MIMAT0000265 | UUCCCUUUGUCAUCCUAUGCCU | 84 |
| miR-205 | MIMAT0000266 | UCCUUCAUUCCACCGGAGUCUG | 85 |
| miR-214 | MIMAT0000271 | ACAGCAGGCACAGACAGGCAGU | 86 |
| miR-215 | MIMAT0000272 | AUGACCUAUGAAUUGACAGAC | 87 |
| miR-218-1 | MIMAT0000275 | UUGUGCUUGAUCUAACCAUGU | 88 |
| miR-218-2 | | | |
| miR-222 | MIMAT0000279 | AGCUACAUCUGGCUACUGGGUCUC | 89 |
| miR-223 | MIMAT0000280 | UGUCAGUUUGUCAAAUACCCC | 90 |
| miR-224 | MIMAT0000281 | CAAGUCACUAGUGGUUCCGUU | 91 |
| miR-301a | MIMAT0000688 | CAGUGCAAUAGUAUUGUCAAAGC | 92 |
| miR-301b | MIMAT0004958 | CAGUGCAAUGAUAUUGUCAAAGC | 93 |
| miR-320a | MIMAT0000510 | AAAAGCUGGGUUGAGAGGGCGA | 94 |
| miR-320b | MIMAT0005792 | AAAAGCUGGGUUGAGAGGGCAA | 95 |
| miR-320c | MIMAT0005793 | AAAAGCUGGGUUGAGAGGGU | 96 |
| miR-320d | MIMAT0006764 | AAAAGCUGGGUUGAGAGGA | 97 |
| miR-324-5p | MIMAT0000761 | CGCAUCCCCUAGGGCAUUGGUGU | 98 |
| miR-324-3p | MIMAT0000762 | ACUGCCCCAGGUGCUGCUGG | 99 |
| miR-326 | MIMAT0000756 | CCUCUGGGCCCUUCCUCCAG | 100 |
| miR-328 | MIMAT0000752 | CUGGCCCUCUCUGCCCUUCCGU | 101 |
| miR-331-3p | MIMAT0000760 | GCCCCUGGGCCUAUCCUAGAA | 102 |
| miR-331-5p | MIMAT0004700 | CUAGGUAUGGUCCCAGGGAUCC | 103 |
| miR-335 | MIMAT0000765 | UCAAGAGCAAUAACGAAAAAUGU | 104 |
| miR-361 | MIMAT0000703 | UUAUCAGAAUCUCCAGGGGUAC | 105 |
| miR-362-5p | MIMAT0000705 | AAUCCUUGGAACCUAGGUGUGAGU | 106 |
| miR-362-3p | MIMAT0004683 | AACACACCUAUUCAAGGAUUCA | 107 |
| miR-365 | MIMAT0000710 | UAAUGCCCCUAAAAAUCCUUAU | 108 |
| miR-375 | MIMAT0000728 | UUUGUUCGUUCGGCUCGCGUGA | 109 |
| miR-381 | MIMAT0000736 | UAUACAAGGGCAAGCUCUCUGU | 110 |
| miR-383 | MIMAT0000738 | AGAUCAGAAGGUGAUUGUGGCU | 111 |
| miR-411 | MIMAT0003329 | UAGUAGACCGUAUAGCGUACG | 112 |
| miR-422b | MIMAT0000732 | CUGGACUUGGAGUCAGAAGGCC | 113 |
| miR-425 | MIMAT0003393 | AAUGACACGAUCACUCCCGUUGA | 114 |
| miR-432 | MIMAT0002814 | UCUUGGAGUAGGUCAUUGGGUGG | 115 |
| miR-451 | MIMAT0001631 | AAACCGUUACCAUUACUGAGUUU | 116 |
| miR-452 | MIMAT0001635 | AACUGUUUGCAGAGGAAACUGA | 117 |
| miR-483 | MIMAT0002173 | UCACUCCUCUCCUCCCGUCUUCU | 118 |
| miR-486-5p | MIMAT0002177 | UCCUGUACUGAGCUGCCCCGAG | 119 |
| miR-486-3p | MIMAT0004762 | CGGGGCAGCUCAGUACAGGAU | 120 |
| miR-487b | MIMAT0003180 | AAUCGUACAGGGUCAUCCACUU | 121 |
| miR-492 | MIMAT0002812 | AGGACCUGCGGGACAAGAUUCUU | 122 |
| miR-497 | MIMAT0002820 | CAGCAGCACACUGUGGUUUGU | 123 |
| miR-501-5p | MIMAT0002872 | AAUCCUUUGUCCCUGGGUGAGA | 124 |
| miR-501-3p | MIMAT0004774 | AAUGCACCCGGGCAAGGAUUCU | 125 |
| miR-515-5p | MIMAT0002826 | UUCUCCAAAAGAAAGCACUUUCUG | 126 |
| miR-516-5p | MIMAT0002859 | CAUCUGGAGGUAAGAAGCACUUU | 127 |
| miR-518b | MIMAT0002844 | CAAAGCGCUCCCCUUUAGAGGU | 128 |
| miR-519d | MIMAT0002853 | CAAAGUGCCUCCCUUUAGAGUGU | 129 |
| miR-524 | MIMAT0002850 | GAAGGCGCUUCCCUUUGGAGU | 130 |
| miR-526b | MIMAT0002835 | CUCUUGAGGGAAGCACUUUCUGUU | 131 |
| miR-532-5p | MIMAT0002888 | CAUGCCUUGAGUGUAGGACCGU | 132 |
| miR-532-3p | MIMAT0004780 | CCUCCCACACCCAAGGCUUGCA | 133 |
| miR-615-5p | MIMAT0004804 | GGGGGUCCCCGGUGCUCGGAUC | 134 |
| miR-615-3p | MIMAT0003283 | UCCGAGCCUGGGUCUCCCUCUU | 135 |
| miR-660 | MIMAT0003338 | UACCCAUUGCAUAUCGGAGUUG | 136 |
| Let-7a | MIMAT0000062 | UGAGGUAGUAGGUUGUAUAGUU | 137 |
| Let-7b | MIMAT0000063 | UGAGGUAGUAGGUUGUGUGGUU | 138 |
| Let-7c | MIMAT0000064 | UGAGGUAGUAGGUUGUAUGGUU | 139 |
| Let-7d | MIMAT0000065 | AGAGGUAGUAGGUUGCAUAGU | 140 |
| Let-7e | MIMAT0000066 | UGAGGUAGGAGGUUGUAUAGU | 141 |
| Let-7f | MIMAT0000067 | UGAGGUAGUAGAUUGUAUAGUU | 142 |
| Let-7g | MIMAT0000414 | UGAGGUAGUAGUUUGUACAGUU | 143 |
| Let-7i | MIMAT0000415 | UGAGGUAGUAGUUUGUGCUGU | 144 |

TABLE 3
List of Significant Genes for Delta = 0.313
Significance Analysis of Microarray results: The microRNA expression profile
of healthy skin was compared with that of atopic eczema samples

| | | | | | | | |
|---|---|---|---|---|---|---|---|
| Row | Gene ID | Gene Name | Score(d) | Numerator(r) | Denominator(s + s0) | Fold Change | q-value[%] |

Positive genes (40)

| | | | | | | | |
|---|---|---|---|---|---|---|---|
| 36 | hsa-miR-146a | hsa-miR-146a | 2.847800754 | 0.75427671 | 0.26486253 | 1.68126002 | 0 |
| 14 | hsa-let-7i | hsa-let-7 [symbol] | 2.504257309 | 1.01799114 | 0.406504211 | 2.03525559 | 0 |
| 77 | hsa-miR-29a | hsa-miR-29a | 2.391953087 | 0.87786575 | 0.367007928 | 1.82824673 | 0 |
| 68 | hsa-miR-222 | hsa-miR-222 | 2.218965456 | 0.7549702 | 0.340235218 | 1.66873008 | 0 |
| 72 | hsa-miR-24 | hsa-miR-24 | 2.195206472 | 0.91806404 | 0.418213073 | 1.85248263 | 0 |
| 46 | hsa-miR-193a | hsa-miR-193a | 2.137919416 | 1.3278388 | 0.621089263 | 2.2672356 | 0 |
| 52 | hsa-miR-199a | hsa-miR-199a | 2.128125085 | 0.79753185 | 0.374757975 | 1.72424482 | 0 |
| 75 | hsa-miR-27a | hsa-miR-27a | 2.040894894 | 0.79882624 | 0.391409788 | 1.71657941 | 0 |
| 6 | hsa-miR-21 | hsa-miR-21 | 2.038000493 | 1.81383848 | 0.890008853 | 3.26259701 | 0 |
| 63 | hsa-miR-20a | hsa-miR-20a | 2.030996142 | 1.14949948 | 0.565978171 | 2.42976149 | 0 |
| 55 | hsa-miR-20a | hsa-miR-20a | 2.013823139 | 1.05659958 | 0.524673522 | 2.27959077 | 0 |
| 37 | hsa-miR-146a | hsa-miR-146a | 1.84420326 | 1.26278817 | 0.684733725 | 2.74918966 | 3.5971223 |
| 128 | hsa-miR-17-5p | hsa-miR-17-5p | 1.824869564 | 1.34047332 | 0.734558431 | 2.58146493 | 3.5971223 |
| 19 | hsa-miR-106b | hsa-miR-106b | 1.783402935 | 0.74734876 | 0.419057718 | 1.72170737 | 3.5971223 |
| 34 | hsa-miR-142-3p | hsa-miR-142-3p | 1.749243431 | 1.63454499 | 0.934429685 | 3.43157103 | 3.5971223 |
| 129 | hsa-miR-30e-5p | hsa-miR-30e-5p | 1.698767753 | 1.18117784 | 0.695314493 | 2.47696251 | 3.5971223 |
| 20 | hsa-miR-107 | hsa-miR-107 | 1.665502598 | 0.57416694 | 0.344740945 | 1.4713302 | 5.1798561 |
| 56 | hsa-miR-200a | hsa-miR-200a | 1.624346538 | 0.84227346 | 0.518530645 | 1.72555934 | 5.1798561 |
| 8 | hsa-miR-146b | hsa-miR-146b | 1.611926434 | 0.65266365 | 0.404896671 | 1.56077801 | 5.1798561 |
| 130 | hsa-miR-27b | hsa-miR-27b | 1.61081782 | 0.842592 | 0.523083361 | 1.70489957 | 5.1798561 |
| 54 | hsa-miR-199b | hsa-miR-199b | 1.486486805 | 0.77152341 | 0.519024729 | 1.70722886 | 5.1798561 |
| 18 | hsa-miR-106a | hsa-miR-106a | 1.431371335 | 0.7234806 | 0.505445778 | 1.57999243 | 7.4709463 |
| 17 | hsa-miR-101 | hsa-miR-101 | 1.33361555 | 0.54101869 | 0.405678147 | 1.43945942 | 8.9307864 |
| 121 | hsa-miR-451 | hsa-miR-451 | 1.277468807 | 0.96044135 | 0.751831546 | 1.94272973 | 10.116906 |
| 28 | hsa-miR-130a | hsa-miR-130a | 1.25887724 | 0.48144493 | 0.382439938 | 1.37623735 | 10.116906 |
| 100 | hsa-miR-451 | hsa-miR-451 | 1.227893705 | 0.65341446 | 0.532142529 | 1.60701289 | 11.7724 |
| 33 | hsa-miR-141 | hsa-miR-141 | 1.191641058 | 0.82780381 | 0.694675468 | 1.91357827 | 13.330512 |
| 84 | hsa-miR-31 | hsa-miR-31 | 1.126639065 | 0.8099326 | 0.718892698 | 1.59359516 | 14.388489 |
| 60 | hsa-miR-203 | hsa-miR-203 | 1.072776216 | 1.66284155 | 1.549849 [symbol] 8 | 1.87078608 | 17.499514 |
| 69 | hsa-miR-223 | hsa-miR-223 | 1.026156621 | 0.53151074 | 0.517962594 | 1.47626816 | 22.07325 |
| 53 | hsa-miR-199a* | hsa-miR-199a* | 1.006190984 | 0.76241748 | 0.757726405 | 1.50424222 | 22.07325 |
| 58 | hsa-miR-200c | hsa-miR-200c | 1.001098271 | 0.72725498 | 0.726457129 | 1.48498483 | 22.07325 |
| 40 | hsa-miR-155 | hsa-miR-155 | 0.96894048 | 0.67676966 | 0.698463605 | 1.42764502 | 23.021583 |
| 43 | hsa-miR-16 | hsa-miR-16 | 0.888957791 | 0.68309207 | 0.768419013 | 1.39490375 | 23.021583 |
| 138 | hsa-miR-361 | hsa-miR-351 | 0.873353389 | 0.45010074 | 0.515370693 | 1.37926987 | 27.39347 |
| 61 | hsa-miR-205 | hsa-miR-205 | 0.830984822 | 0.99314164 | 1.195138122 | 1.46811545 | 27.39347 |
| 131 | hsa-miR-143 | hsa-miR-143 | 0.828695815 | 0.48428817 | 0.58439799 | 1.28262814 | 27.39347 |
| 97 | hsa-miR-422b | hsa-miR-422b | 0.806410934 | 0.31844853 | 0.394896092 | 1.21690006 | 27.39347 |
| 57 | hsa-miR-200b | hsa-miR-200b | 0.804645158 | 0.53430154 | 0.664021328 | 1.32222337 | 27.39347 |
| 42 | hsa-miR-15b | hsa-miR-15b | 0.794999084 | 0.43930235 | 0.552582212 | 1.26155122 | 27.39347 |

Negative genes (12)

| | | | | | | | |
|---|---|---|---|---|---|---|---|
| 23 | hsa-miR-122a | hsa-miR-122a | −2.749245741 | −2.24837574 | 0.817815487 | 0.1878036 | 0 |
| 30 | hsa-miR-133a-133b | hsa-miR-133a-133b | −2.732418887 | −1.6955356 | 0.620525501 | 0.279006 | 0 |
| 87 | hsa-miR-326 | hsa-miR-326 | −2.50117378 | −1.27659774 | 0.510399456 | 0.39304256 | 0 |
| 65 | hsa-miR-215 | hsa-miR-215 | −2.481879021 | −1.21822966 | 0.490849736 | 0.42039862 | 0 |
| 132 | hsa-miR-483 | hsa-miR-483 | −1.885807135 | −0.86536677 | 0.458884028 | 0.56944972 | 8.9307864 |
| 115 | hsa-miR-519d | hsa-miR-519d | −1.861389454 | −1.03229943 | 0.554585406 | 0.47559914 | 8.9307864 |
| 89 | hsa-miR-335 | hsa-miR-335 | −1.776102342 | −0.64853741 | 0.365146418 | 0.6312991 | 10.116906 |
| 31 | hsa-miR-133b | hsa-miR-133b | −1.683955284 | −1.65823882 | 0.984728535 | 0.2270142 | 12.94964 |
| 112 | hsa-miR-515-5p | hsa-miR-515-5p | −1.566990919 | −1.05110262 | 0.670777734 | 0.43240628 | 17.371469 |
| 48 | hsa-miR-194 | hsa-miR-194 | −1.506287708 | −1.04653859 | 0.694780012 | 0.41926544 | 17.371469 |
| 10 | hsa-miR-524* | hsa-miR-524* | −1.487588872 | −0.596361 | 0.400891009 | 0.66386618 | 17.371469 |
| 50 | hsa-miR-197 | hsa-miR-197 | −1.461762755 | −0.61234898 | 0.418911333 | 0.6606391 | 17.371469 |

[symbol]

indicates data missing or illegible when filed

TABLE 4

| Gene Name | Score(d) | Numerator(r) | Denominator(s + s0) | Fold Change | q-value(%) |
|---|---|---|---|---|---|
| hsa-miR-142-3p-4373136 | 4.378755 | 1.607882 | 0.367201 | 3.084592 | 0 |
| hsa-miR-155-4373124 | 4.045666 | 2.201913 | 0.544265 | 4.622148 | 0 |
| hsa-miR-31-4373190 | 4.045172 | 2.949023 | 0.729023 | 8.503418 | 0 |
| hsa-miR-135b-4373139 | 3.97071 | 2.075938 | 0.522813 | 4.161518 | 0 |
| hsa-miR-223-4373075 | 3.024304 | 2.567422 | 0.84893 | 7.796735 | 0.95264 |
| hsa-miR-187-4373111 | 2.864714 | 2.313972 | 0.80775 | 5.282102 | 0.95264 |
| hsa-miR-142-5p-4373135 | 2.496498 | 1.454419 | 0.582584 | 2.870536 | 3.104901 |
| hsa-miR-501-4373226 | 2.421184 | 2.028121 | 0.837657 | 3.142973 | 3.104901 |
| hsa-miR-487b-4378102 | 2.411741 | 0.890143 | 0.369087 | 1.823577 | 3.104901 |
| hsa-miR-362-4378092 | 2.40528 | 0.898964 | 0.373746 | 1.81384 | 3.104901 |
| hsa-miR-301-4373064 | 2.063725 | 0.84708 | 0.410462 | 1.837104 | 5.408538 |
| hsa-miR-130b-4373144 | 2.039959 | 1.009641 | 0.494932 | 1.976644 | 5.408538 |
| hsa-miR-146b-4373178 | 2.02866 | 1.288674 | 0.635234 | 2.852587 | 5.408538 |

TABLE 5

| Gene Name | Score(d) | Numerator(r) | Denominator(s + s0) | Fold Change | q-value(%) |
|---|---|---|---|---|---|
| hsa-miR-99a-4373008 | −6.04738 | −2.0183 | 0.333748 | 0.248652 | 0 |
| hsa-miR-383-4373018 | −4.77089 | −3.18162 | 0.666883 | 0.095693 | 0 |
| hsa-miR-125b-4373148 | −3.87755 | −1.56497 | 0.403597 | 0.344311 | 0 |
| hsa-miR-193b-4373185 | −3.84871 | −2.16266 | 0.561918 | 0.244698 | 0 |
| hsa-miR-10a-4373153 | −3.81751 | −2.06867 | 0.541889 | 0.225324 | 0 |
| hsa-miR-30a-3p-4373062 | −3.81637 | −1.22445 | 0.320841 | 0.433119 | 0 |
| hsa-miR-100-4373160 | −3.80818 | −1.5392 | 0.404182 | 0.359408 | 0 |
| hsa-miR-328-4373049 | −3.77431 | −1.87846 | 0.497698 | 0.288528 | 0 |
| hsa-miR-375-4373027 | −3.65756 | −2.75268 | 0.7526 | 0.164098 | 0 |
| hsa-let-7c-4373167 | −3.63714 | −1.29772 | 0.356798 | 0.412749 | 0 |
| hsa-miR-149-4373128 | −3.5306 | −1.75469 | 0.496996 | 0.313165 | 0 |
| hsa-let-7a-4373169 | −3.49817 | −1.26355 | 0.361203 | 0.415284 | 0 |
| hsa-miR-130a-4373145 | −3.3135 | −0.75955 | 0.229227 | 0.593078 | 0 |
| hsa-miR-196b-4373103 | −3.27877 | −1.12147 | 0.342039 | 0.472103 | 0 |
| hsa-miR-197-4373102 | −3.14883 | −0.97294 | 0.308985 | 0.505505 | 0 |
| hsa-miR-26b-4373069 | −3.13817 | −0.88531 | 0.282111 | 0.541842 | 0 |
| hsa-miR-30e-3p-4373057 | −3.03191 | −1.06231 | 0.350376 | 0.485263 | 0 |
| hsa-miR-214-4373085 | −2.85054 | −0.99412 | 0.348746 | 0.506723 | 0 |
| hsa-let-7b-4373168 | −2.75513 | −0.91065 | 0.330529 | 0.530145 | 0 |
| hsa-miR-26a-4373070 | −2.74665 | −0.94222 | 0.343042 | 0.527914 | 0 |
| hsa-miR-101-4373159 | −2.73899 | −1.29598 | 0.47316 | 0.43303 | 0 |
| hsa-miR-30c-4373060 | −2.69194 | −0.71693 | 0.266323 | 0.606057 | 0 |
| hsa-miR-195-4373105 | −2.65536 | −1.13527 | 0.427539 | 0.477336 | 0 |
| hsa-let-7f-4373164 | −2.62864 | −0.99246 | 0.377557 | 0.492843 | 0 |
| hsa-miR-148a-4373130 | −2.5669 | −0.86253 | 0.336019 | 0.560532 | 1.397206 |
| hsa-miR-125a-4373149 | −2.55992 | −0.8137 | 0.317859 | 0.565006 | 1.397206 |
| hsa-miR-30e-5p-4373058 | −2.40463 | −0.76863 | 0.319647 | 0.596565 | 1.397206 |
| hsa-miR-29c-4373289 | −2.31281 | −0.68918 | 0.297985 | 0.624465 | 1.397206 |
| hsa-miR-335-4373045 | −2.30675 | −1.03751 | 0.449769 | 0.466339 | 1.397206 |
| hsa-miR-199b-4373100 | −2.24244 | −1.01818 | 0.454052 | 0.523841 | 1.397206 |
| hsa-miR-365-4373194 | −2.17202 | −1.24449 | 0.572964 | 0.464332 | 1.397206 |
| hsa-let-7d-4373166 | −2.02807 | −1.00011 | 0.493137 | 0.510683 | 1.397206 |
| hsa-miR-204-4373094 | −1.99469 | −1.45447 | 0.729173 | 0.350249 | 1.397206 |
| hsa-miR-23a-4373074 | −1.98014 | −1.08338 | 0.547122 | 0.510837 | 1.397206 |
| hsa-miR-196a-4373104 | −1.95493 | −1.35207 | 0.691622 | 0.452161 | 1.397206 |
| hsa-miR-452-4378077 | −1.94633 | −1.07419 | 0.551906 | 0.520588 | 1.397206 |
| hsa-miR-143-4373134 | −1.91854 | −1.01914 | 0.531203 | 0.479496 | 1.397206 |
| hsa-miR-199a-4378068 | −1.91765 | −0.75679 | 0.394645 | 0.590972 | 1.397206 |
| hsa-miR-145-4373133 | −1.87997 | −0.88982 | 0.473314 | 0.507434 | 1.397206 |
| hsa-miR-23b-4373073 | −1.87852 | −0.92947 | 0.494788 | 0.563197 | 1.397206 |
| hsa-miR-486-4378096 | −1.64369 | −1.68641 | 1.025988 | 0.265357 | 3.104901 |
| hsa-miR-92-4373013 | −1.63478 | −0.79137 | 0.484082 | 0.598864 | 3.104901 |
| hsa-miR-615-4380991 | −1.60544 | −1.57382 | 0.980301 | 0.445292 | 3.104901 |
| hsa-miR-497-4373222 | −1.59269 | −0.72665 | 0.456242 | 0.613462 | 3.104901 |
| hsa-miR-99b-4373007 | −1.55486 | −0.71492 | 0.459797 | 0.624682 | 3.810561 |
| hsa-miR-9-4373285 | −1.40531 | −1.01605 | 0.723009 | 0.46856 | 5.058848 |
| hsa-miR-10b-4373152 | −1.35584 | −0.77149 | 0.569011 | 0.620912 | 5.058848 |
| hsa-miR-1-4373161 | −1.28859 | −0.999 | 0.775269 | 0.403283 | 5.058848 |
| hsa-miR-451-4373209 | −1.11603 | −1.08736 | 0.974303 | 0.392322 | 5.408538 |

TABLE 6
List of Significant Genes for Delta = 0.497
Significance Analysis of Microarray results: The microRNA expression profile
of healthy skin was compared with that of psoriasis samples

| | | | | | | | |
|---|---|---|---|---|---|---|---|
| Row | Gene ID | Gene Name | Score(d) | Numerator(r) | Denominator(s + s0) | Fold Change | q-value(%) |

Postive genes (25)

| | | | | | | | |
|---|---|---|---|---|---|---|---|
| 55 | hsa-miR-2 [symbol] | hsa-miR-20a | 5.170411954 | 1.62447184 | 0.314186152 | 3.14945009 | 0 |
| 36 | hsa-miR-1 [symbol] | hsa-miR-146a | 5.058578965 | 1.57115956 | 0.310593069 | 2.92702779 | 0 |
| 8 | hsa-miR-1 [symbol] | hsa-miR-146b | 4.675413034 | 1.74589905 | 0.373421353 | 3.31096122 | 0 |
| 84 | hsa-miR-3 [symbol] | hsa-miR-31 | 3.473376049 | 2.36599204 | 0.681179351 | 4.68651364 | 0 |
| 37 | hsa-miR-1 [symbol] | hsa-miR-146a | 3.040941498 | 1.87952335 | 0.618072841 | 3.66798762 | 0 |
| 63 | hsa-miR-2 [symbol] | hsa-miR-20a | 2.990800951 | 1.39302887 | 0.465771174 | 2.64322314 | 0 |
| 56 | hsa-miR-2 [symbol] | hsa-miR-200a | 2.854728326 | 1.5347068 | 0.537601699 | 2.7493251 | 0 |
| 128 | hsa-miR-1 [symbol] | hsa-miR-17-5p | 2.67025785 | 1.89194925 | 0.708526799 | 3.77246059 | 0 |
| 129 | hsa-miR-3 [symbol] | hsa-miR-30e-5p | 2.611400855 | 1.79358072 | 0.686827042 | 3.61090198 | 0 |
| 33 | hsa-miR-1 [symbol] | hsa-miR-141 | 2.264261118 | 1.61882182 | 0.714944847 | 3.44625503 | 3.4009156 |
| 60 | hsa-miR-2 [symbol] | hsa-miR-203 | 2.227372204 | 3.31926781 | 1.490216948 | 5.86052607 | 3.4009156 |
| 34 | hsa-miR-1 [symbol] | hsa-miR-142-3p | 2.216758209 | 1.51428402 | 0.683107437 | 2.55218154 | 3.4009156 |
| 6 | hsa-miR-2 [symbol] | hsa-miR-21 | 2.209169714 | 1.60495806 | 0.726498308 | 2.50634692 | 3.4009156 |
| 18 | hsa-miR-1 [symbol] | hsa-miR-106a | 2.163687633 | 1.22347959 | 0.565460363 | 2.36519222 | 3.4009156 |
| 135 | hsa-miR-4 [symbol] | hsa-miR-487b | 2.055234968 | 0.8970352 | 0.436463573 | 1.87341258 | 5.1600099 |
| 41 | hsa-miR-1 [symbol] | hsa-miR-15a | 1.900393906 | 0.94737634 | 0.498515772 | 1.82226153 | 5.1600099 |
| 14 | hsa-let-7i | hsa-let-7i | 1.897106069 | 0.58663358 | 0.309225506 | 1.48257927 | 5.1600099 |
| 68 | hsa-miR-2 [symbol] | hsa-miR-222 | 1.886268385 | 0.55635819 | 0.294951766 | 1.45156733 | 5.1600099 |
| 97 | hsa-miR-4 | hsa-miR-422b | 1.856882941 | 0.84621964 | 0.455720507 | 1.82463313 | 5.1600099 |
| 28 | hsa-miR-1 [symbol] | hsa-miR-130a | 1.84481411 | 0.67545637 | 0.366137906 | 1.58442701 | 5.1600099 |
| 46 | hsa-miR-1 [symbol] | hsa-miR-193a | 1.695817119 | 1.00563655 | 0.593010026 | 1.82514514 | 7.4820144 |
| 19 | hsa-miR-1 [symbol] | hsa-miR-106b | 1.507570917 | 0.50208526 | 0.333042547 | 1.45237131 | 10.391687 |
| 54 | hsa-miR-1 [symbol] | hsa-miR-199b | 1.42310625 | 0.67341997 | 0.473204279 | 1.58737508 | 14.599052 |
| 53 | hsa-miR-1 [symbol] | hsa-miR-199a* | 1.422388268 | 1.08449603 | 0.762447255 | 1.9419421 | 14.599052 |
| 130 | hsa-miR-2 [symbol] | hsa-miR-27b | 1.36183848 | 0.72696218 | 0.533809397 | 1.61158123 | 16.032888 |

Negative genes (28)

| | | | | | | | |
|---|---|---|---|---|---|---|---|
| 26 | hsa-miR-1 [symbol] | hsa-miR-125b | −5.307902656 | −0.87283027 | 0.164439766 | 0.54581789 | 0 |
| 120 | hsa-miR-9 [symbol] | hsa-miR-99b | −3.319282075 | −0.7876049 | 0.237281702 | 0.58239304 | 0 |
| 23 | hsa-miR-1 [symbol] | hsa-miR-122a | −3.091017016 | −2.29557408 | 0.742659799 | 0.17738672 | 0 |
| 50 | hsa-miR-1 [symbol] | hsa-miR-197 | −2.69162708 | −0.62681495 | 0.232875852 | 0.64415248 | 4.676259 |
| 16 | hsa-miR-1 [symbol] | hsa-miR-100 | −2.621714944 | −0.76668573 | 0.292436723 | 0.58706565 | 4.676259 |
| 94 | hsa-miR-3 [symbol] | hsa-miR-381 | −2.600891984 | −0.487246 | 0.187338037 | 0.71214905 | 4.676259 |
| 114 | hsa-miR-5 [symbol] | hsa-miR-518b | −2.545067041 | −0.84176382 | 0.330743278 | 0.56394914 | 4.676259 |
| 10 | hsa-miR-5 [symbol] | hsa-miR-524* | −2.397262458 | −1.05228285 | 0.438951874 | 0.49914806 | 4.676259 |
| 126 | hsa-let-7e | hsa-let-7e | −2.248140883 | −0.75991931 | 0.338021214 | 0.60050376 | 5.1600099 |
| 82 | hsa-miR-3 [symbol] | hsa-miR-30c | −1.982757588 | −0.64015809 | 0.322862509 | 0.63160564 | 12.067765 |
| 90 | hsa-miR-3 [symbol] | hsa-miR-365 | −1.868138265 | −0.6624109 | 0.354583442 | 0.61714601 | 12.067765 |
| 31 | hsa-miR-1 [symbol] | hsa-miR-133b | −1.783954708 | −1.69990138 | 0.952883707 | 0.22302786 | 12.067765 |
| 21 | hsa-miR-1 [symbol] | hsa-miR-10a | −1.776839036 | −0.58901933 | 0.331498419 | 0.66764029 | 12.067765 |
| 30 | hsa-miR-1 [symbol] | hsa-miR-133a-133b | −1.757675811 | −1.50215643 | 0.854626557 | 0.40459161 | 12.067765 |
| 66 | hsa-miR-2 [symbol] | hsa-miR-22 | −1.680288261 | −0.66312364 | 0.394648737 | 0.61419675 | 12.067765 |
| 87 | hsa-miR-3 [symbol] | hsa-miR-326 | −1.610882797 | −0.78233891 | 0.485658495 | 0.55702207 | 14.599052 |
| 65 | hsa-miR-2 [symbol] | hsa-miR-215 | −1.592738733 | −0.87139451 | 0.547104491 | 0.56299509 | 14.599052 |
| 113 | hsa-miR-5 [symbol] | hsa-miR-516-5p | −1.461293425 | −0.37861039 | 0.259092652 | 0.76965012 | 18.705036 |
| 12 | hsa-let-7c | hsa-let-7c | −1.439174915 | −0.68241616 | 0.474171799 | 0.60965569 | 18.705036 |
| 13 | hsa-let-7d | hsa-let-7d | −1.43845147 | −0.37713491 | 0.262181187 | 0.76637288 | 18.705036 |
| 89 | hsa-miR-3 [symbol] | hsa-miR-335 | −1.411689579 | −0.80761774 | 0.572093013 | 0.63146066 | 18.705036 |
| 103 | hsa-miR-4 [symbol] | hsa-miR-492 | −1.400491698 | −0.38617242 | 0.275740601 | 0.75733855 | 18.705036 |
| 15 | hsa-miR-1 [symbol] | hsa-miR-1 | −1.386855692 | −1.40056087 | 1.009882193 | 0.2602516 | 18.705036 |
| 115 | hsa-miR-5 [symbol] | hsa-miR-519d | −1.278039174 | −0.52748368 | 0.412728879 | 0.66876353 | 21.175512 |
| 22 | hsa-miR-1 [symbol] | hsa-miR-10b | −1.275506037 | −0.53978559 | 0.423193286 | 0.70088255 | 21.175512 |
| 132 | hsa-miR-4 [symbol] | hsa-miR-483 | −1.260685484 | −0.35279829 | 0.279846397 | 0.77645168 | 21.175512 |
| 48 | hsa-miR-1 [symbol] | hsa-miR-194 | −1.250780026 | −0.8234822 | 0.65837492 | 0.48999421 | 21.175512 |
| 117 | hsa-miR-5 [symbol] | hsa-miR-526b | −1.214089964 | −0.46597639 | 0.383807136 | 0.74282263 | 21.175512 |

[symbol]

indicates data missing or illegible when filed

TABLE 7

| Gene Name | Score(d) | Numerator(r) | Denominator(s + s0) | Fold Change | q-value(%) |
|---|---|---|---|---|---|
| hsa-miR-31-4373190 | 11.76748 | 5.83201 | 0.495604 | 53.46021 | 0 |
| hsa-miR-146a-4373132 | 6.157881 | 1.361142 | 0.221041 | 2.550485 | 0 |
| hsa-miR-21-4373090 | 5.690929 | 1.950575 | 0.342752 | 3.77251 | 0 |
| hsa-miR-135b-4373139 | 4.495819 | 2.897555 | 0.6445 | 6.784366 | 0 |
| hsa-miR-205-4373093 | 3.249398 | 0.596745 | 0.183648 | 1.506377 | 1.230214 |
| hsa-miR-142-3p-4373136 | 2.985761 | 0.690889 | 0.231395 | 1.600742 | 1.230214 |
| hsa-miR-155-4373124 | 2.979155 | 1.345772 | 0.451729 | 2.436721 | 1.230214 |
| hsa-miR-223-4373075 | 2.842751 | 0.966022 | 0.339819 | 1.907887 | 1.230214 |
| hsa-miR-17-5p-4373119 | 2.791165 | 0.875423 | 0.313641 | 1.798112 | 1.230214 |
| hsa-miR-93-4373012 | 2.68718 | 1.080648 | 0.402149 | 2.036204 | 1.230214 |
| hsa-miR-203-4373095 | 2.368399 | 0.656687 | 0.27727 | 1.555213 | 1.230214 |
| hsa-miR-132-4373143 | 2.322615 | 0.845088 | 0.363852 | 1.749608 | 1.230214 |
| hsa-miR-425-4373202 | 2.30791 | 1.157098 | 0.501362 | 2.097308 | 1.230214 |
| hsa-miR-362-4378092 | 2.272419 | 0.833846 | 0.366942 | 1.735073 | 1.230214 |
| hsa-miR-324-5p-4373052 | 2.193747 | 0.361533 | 0.164802 | 1.281118 | 1.230214 |
| hsa-miR-224-4373187 | 1.980246 | 1.188235 | 0.600044 | 2.081201 | 1.230214 |
| hsa-miR-432-4373280 | 1.751434 | 1.16155 | 0.663199 | 2.032994 | 1.230214 |
| hsa-miR-301-4373064 | 1.721598 | 0.4905 | 0.28491 | 1.385618 | 1.230214 |
| hsa-miR-20a-4373286 | 1.699744 | 0.41699 | 0.245325 | 1.32226 | 1.230214 |

TABLE 8

| Gene Name | Score(d) | Numerator(r) | Denominator(s + s0) | Fold Change | q-value(%) |
|---|---|---|---|---|---|
| hsa-miR-100-4373160 | −12.7485 | −1.71946 | 0.134875 | 0.303266 | 0 |
| hsa-miR-30a-3p-4373062 | −12.4543 | −2.60401 | 0.209085 | 0.163481 | 0 |
| hsa-miR-195-4373105 | −10.4344 | −1.77231 | 0.169853 | 0.291858 | 0 |
| hsa-miR-30a-5p-4373061 | −10.0142 | −1.41049 | 0.14085 | 0.37559 | 0 |
| hsa-miR-30e-5p-4373058 | −8.79751 | −1.41719 | 0.16109 | 0.373476 | 0 |
| hsa-miR-99a-4373008 | −7.87582 | −1.55446 | 0.197371 | 0.338712 | 0 |
| hsa-miR-193b-4373185 | −7.7373 | −1.6623 | 0.214842 | 0.313894 | 0 |
| hsa-miR-149-4373128 | −7.19907 | −2.06626 | 0.287018 | 0.235224 | 0 |
| hsa-miR-26a-4373070 | −7.12656 | −1.51078 | 0.211993 | 0.348681 | 0 |
| hsa-let-7c-4373167 | −6.77945 | −1.46013 | 0.215376 | 0.361063 | 0 |
| hsa-miR-26b-4373069 | −6.63502 | −1.13863 | 0.171609 | 0.452712 | 0 |
| hsa-miR-218-4373081 | −6.60577 | −1.43134 | 0.21668 | 0.368235 | 0 |
| hsa-miR-30e-3p-4373057 | −6.49389 | −1.4485 | 0.223057 | 0.363718 | 0 |
| hsa-miR-23b-4373073 | −6.44952 | −1.24703 | 0.193352 | 0.419313 | 0 |
| hsa-let-7g-4373163 | −6.36312 | −1.3551 | 0.212961 | 0.388355 | 0 |
| hsa-miR-411-4381013 | −6.23084 | −1.32534 | 0.212707 | 0.396504 | 0 |
| hsa-miR-125b-4373148 | −6.13734 | −1.6646 | 0.271224 | 0.311236 | 0 |
| hsa-miR-199b-4373100 | −6.08363 | −1.19458 | 0.19636 | 0.434804 | 0 |
| hsa-miR-29c-4373289 | −5.46365 | −1.11354 | 0.203809 | 0.459639 | 0 |
| hsa-miR-101-4373159 | −5.27585 | −1.34222 | 0.254409 | 0.390295 | 0 |
| hsa-miR-197-4373102 | −5.18262 | −1.37159 | 0.264651 | 0.381852 | 0 |
| hsa-miR-214-4373085 | −5.06303 | −1.21827 | 0.240621 | 0.425845 | 0 |
| hsa-miR-375-4373027 | −4.99986 | −2.55427 | 0.510869 | 0.160598 | 0 |
| hsa-miR-335-4373045 | −4.916 | −2.21699 | 0.450974 | 0.205585 | 0 |
| hsa-miR-181d-4373180 | −4.83571 | −0.58649 | 0.121284 | 0.665428 | 0 |
| hsa-miR-125a-4373149 | −4.59551 | −1.44142 | 0.313659 | 0.361267 | 0 |
| hsa-miR-140-4373138 | −4.5163 | −1.49654 | 0.331364 | 0.346769 | 0 |
| hsa-miR-30c-4373060 | −4.51423 | −1.00186 | 0.221934 | 0.495636 | 0 |
| hsa-miR-30b-4373290 | −4.45057 | −0.83052 | 0.186609 | 0.559952 | 0 |
| hsa-miR-152-4373126 | −4.37175 | −1.24311 | 0.284351 | 0.416297 | 0 |
| hsa-miR-328-4373049 | −4.27844 | −1.052 | 0.245885 | 0.477677 | 0 |
| hsa-miR-497-4373222 | −4.19971 | −1.3895 | 0.330856 | 0.373094 | 0 |
| hsa-miR-130a-4373145 | −4.17585 | −0.52426 | 0.125546 | 0.694672 | 0 |
| hsa-miR-127-4373147 | −4.09023 | −0.93473 | 0.228529 | 0.518965 | 0 |
| hsa-miR-148b-4373129 | −3.92101 | −0.8323 | 0.212266 | 0.558215 | 0 |
| hsa-miR-186-4373112 | −3.9143 | −0.96048 | 0.245377 | 0.508719 | 0 |
| hsa-miR-143-4373134 | −3.75643 | −1.8063 | 0.480855 | 0.269531 | 0 |
| hsa-miR-145-4373133 | −3.66864 | −1.76478 | 0.481045 | 0.277357 | 0 |
| hsa-miR-30d-4373059 | −3.61196 | −1.06998 | 0.296232 | 0.468453 | 0 |
| hsa-miR-126-4373269 | −3.59126 | −1.26174 | 0.351338 | 0.407415 | 0 |
| hsa-miR-199a-4378068 | −3.58301 | −1.15516 | 0.322399 | 0.439907 | 0 |
| hsa-miR-196b-4373103 | −3.48818 | −0.46805 | 0.134181 | 0.722021 | 0 |
| hsa-miR-486-4378096 | −3.48593 | −3.58037 | 1.02709 | 0.061813 | 0 |
| hsa-miR-99b-4373007 | −3.44326 | −0.9523 | 0.276569 | 0.510162 | 0 |
| hsa-miR-365-4373194 | −3.40403 | −0.97013 | 0.284994 | 0.502766 | 0 |
| hsa-miR-29a-4373065 | −3.35492 | −1.15777 | 0.345095 | 0.436984 | 0 |
| hsa-miR-320-4373055 | −3.28942 | −0.69838 | 0.212311 | 0.61238 | 0 |
| hsa-miR-361-4373035 | −3.24661 | −0.95643 | 0.294594 | 0.507426 | 0 |
| hsa-miR-95-4373011 | −3.12399 | −1.15348 | 0.369234 | 0.437066 | 0 |
| hsa-let-7d-4373166 | −3.05807 | −1.21594 | 0.397615 | 0.417051 | 0 |
| hsa-miR-532-4380928 | −3.02082 | −0.87273 | 0.288905 | 0.537995 | 0 |
| hsa-miR-331-4373046 | −2.96463 | −1.17026 | 0.394742 | 0.428809 | 0 |
| hsa-miR-199a-4373272 | −2.86962 | −0.85379 | 0.297526 | 0.544137 | 0 |
| hsa-miR-196a-4373104 | −2.78127 | −1.04927 | 0.377263 | 0.468517 | 0 |
| hsa-miR-451-4373209 | −2.71471 | −2.73461 | 1.007331 | 0.111021 | 0 |
| hsa-miR-193a-4373107 | −2.71086 | −1.08375 | 0.399782 | 0.455581 | 0 |
| hsa-miR-126-4378064 | −2.64503 | −0.93877 | 0.354917 | 0.509333 | 0 |
| hsa-miR-10a-4373153 | −2.46892 | −1.36565 | 0.553138 | 0.359901 | 0 |
| hsa-miR-23a-4373074 | −2.41463 | −0.74724 | 0.309464 | 0.585067 | 0 |
| hsa-miR-660-4380925 | −2.31775 | −0.53945 | 0.232746 | 0.682082 | 0 |
| hsa-let-7e-4373165 | −2.24737 | −0.99439 | 0.442466 | 0.479835 | 0 |
| hsa-let-7b-4373168 | −2.00893 | −0.58168 | 0.289546 | 0.658749 | 0 |
| hsa-miR-16-4373121 | −1.97327 | −0.99477 | 0.504123 | 0.470933 | 0 |
| hsa-miR-182-4373271 | −1.96757 | −0.70994 | 0.36082 | 0.595922 | 0 |
| hsa-miR-1-4373161 | −1.91902 | −1.60722 | 0.837523 | 0.264057 | 0 |
| hsa-miR-22-4373079 | −1.83572 | −0.6217 | 0.338668 | 0.634454 | 0.521376 |
| hsa-miR-27a-4373287 | −1.79766 | −0.45225 | 0.251576 | 0.723423 | 0.521376 |
| hsa-miR-383-4373018 | −1.76341 | −1.26552 | 0.717654 | 0.358066 | 0.9999 |
| hsa-let-7a-4373169 | −1.75317 | −0.52559 | 0.299796 | 0.682696 | 0.9999 |
| hsa-miR-191-4373109 | −1.66002 | −0.65045 | 0.391831 | 0.614944 | 1.459854 |

TABLE 9
List of Significant Genes for Delta = 0.396
Significance Analysis of Microarray results: The microRNA expression profile of healthy skin
was compared with the united group of atopic eczema and psoriasis samples (inflammation).

| | | | | | | | |
|---|---|---|---|---|---|---|---|
| Row | Gene ID | Gene Name | Score(d) | Numerator(r) | Denominator(s + s0) | Fold Change | q-value(%) |

Positive genes (36)

| | | | | | | | |
|---|---|---|---|---|---|---|---|
| 55 | hsa-miR-20a | hsa-miR-20a | 2.905315198 | 1.34053576 | 0.461408029 | 2.71452043 | 0 |
| 36 | hsa-miR-146a | hsa-miR-146a | 2.770957363 | 1.31199403 | 0.47348041 | 2.64398374 | 0 |
| 63 | hsa-miR-20a | hsa-miR-20a | 2.65856949 | 1.28401942 | 0.482957916 | 2.58040886 | 0 |
| 84 | hsa-miR-31 | hsa-miR-31 | 2.65571483 | 1.8038398 | 0.679229478 | 3.44146864 | 0 |
| 6 | hsa-miR-21 | hsa-miR-21 | 2.652995661 | 1.70939827 | 0.644327579 | 2.88447197 | 0 |
| 128 | hsa-miR-17-5p | hsa-miR-17-5p | 2.547591685 | 1.61621128 | 0.634407505 | 3.17696276 | 0 |
| 46 | hsa-miR-193a | hsa-miR-193a | 2.436138517 | 1.16673767 | 0.478929119 | 2.04619037 | 0 |
| 8 | hsa-miR-146b | hsa-miR-146b | 2.416960169 | 1.21513647 | 0.502754033 | 2.45394547 | 0 |
| 37 | hsa-miR-146a | hsa-miR-146a | 2.415597859 | 1.37355695 | 0.568619873 | 2.49229018 | 0 |
| 68 | hsa-miR-222 | hsa-miR-222 | 2.335412904 | 0.65566419 | 0.280748724 | 1.5601487 | 0 |
| 34 | hsa-miR-142-3p | hsa-miR-142-3p | 2.291851252 | 1.5744145 | 0.686961906 | 2.99187629 | 0 |
| 14 | hsa-let-7i | hsa-let-7i | 2.269720024 | 0.80231236 | 0.353485168 | 1.75891743 | 0 |
| 18 | hsa-miR-106a | hsa-miR-106a | 2.218340153 | 1.03354189 | 0.46590776 | 2.07247275 | 0 |
| 56 | hsa-miR-200a | hsa-miR-200a | 2.175172577 | 1.12640654 | 0.517846975 | 2.13854216 | 0 |
| 129 | hsa-miR-30e-5p | hsa-miR-30e-5p | 2.093551785 | 1.40340449 | 0.670346202 | 2.89347513 | 0 |
| 60 | hsa-miR-203 | hsa-miR-203 | 2.03328799 | 2.49095468 | 1.225086998 | 3.86565608 | 0 |
| 130 | hsa-miR-27b | hsa-miR-27b | 1.834318325 | 0.78477709 | 0.42783037 | 1.6582404 | 0 |
| 33 | hsa-miR-141 | hsa-miR-141 | 1.791636261 | 1.22331281 | 0.682790833 | 2.67991665 | 2.413553 |
| 28 | hsa-miR-130a | hsa-miR-130a | 1.769327429 | 0.57845065 | 0.326932508 | 1.48033218 | 2.413553 |
| 72 | hsa-miR-24 | hsa-miR-24 | 1.703628092 | 0.67452023 | 0.395931617 | 1.59498406 | 2.413553 |
| 54 | hsa-miR-199b | hsa-miR-199b | 1.693170981 | 0.72247169 | 0.42669742 | 1.64730197 | 2.413553 |
| 52 | hsa-miR-199a | hsa-miR-199a | 1.640866609 | 0.5632198 | 0.34324533 | 1.48094868 | 2.413553 |
| 19 | hsa-miR-106b | hsa-miR-106b | 1.640332115 | 0.52149369 | 0.317919577 | 1.4572494 | 2.413553 |
| 53 | hsa-miR-199a* | hsa-miR-199a* | 1.557072626 | 0.92345675 | 0.593072371 | 1.72309216 | 2.413553 |
| 41 | hsa-miR-15a | hsa-miR-15a | 1.475571462 | 0.66594758 | 0.45131503 | 1.52312451 | 6.235012 |
| 121 | hsa-miR-451 | hsa-miR-451 | 1.464462114 | 0.88975906 | 0.607567144 | 1.84810235 | 6.235012 |
| 77 | hsa-miR-29a | hsa-miR-29a | 1.426987269 | 0.56297049 | 0.394516826 | 1.50961726 | 6.235012 |
| 97 | hsa-miR-422b | hsa-miR-422b | 1.399326098 | 0.58233408 | 0.416153234 | 1.5207666 | 6.235012 |
| 61 | hsa-miR-205 | hsa-miR-205 | 1.378585252 | 1.1696045 | 0.848409264 | 1.65259237 | 6.235012 |
| 138 | hsa-miR-361 | hsa-miR-361 | 1.2210991 | 0.49461286 | 0.405055463 | 1.39879716 | 11.510791 |
| 135 | hsa-miR-487b | hsa-miR-487b | 1.218967281 | 0.53997635 | 0.442978541 | 1.49422845 | 11.510791 |
| 20 | hsa-miR-107 | hsa-miR-107 | 1.155915545 | 0.40506566 | 0.350428421 | 1.34219172 | 13.467626 |
| 100 | hsa-miR-451 | hsa-miR-451 | 1.146530723 | 0.55210614 | 0.481545001 | 1.51714886 | 13.467626 |
| 43 | hsa-miR-16 | hsa-miR-16 | 0.960909677 | 0.56733408 | 0.590413535 | 1.31307339 | 22.31478 |
| 75 | hsa-miR-27a | hsa-miR-27a | 0.938165609 | 0.4120479 | 0.439205933 | 1.36792156 | 22.31478 |
| 40 | hsa-miR-155 | hsa-miR-155 | 0.869422986 | 0.45825653 | 0.527081222 | 1.23399018 | 25.80005 |

Negative genes (22)

| | | | | | | | |
|---|---|---|---|---|---|---|---|
| 23 | hsa-miR-122a | hsa-miR-122a | −3.683826842 | −2.27197491 | 0.616743134 | 0.18259516 | 0 |
| 30 | hsa-miR-133a-133b | hsa-miR-133a-133b | −2.496214028 | −1.59884601 | 0.640508384 | 0.3417988 | 0 |
| 50 | hsa-miR-197 | hsa-miR-197 | −2.431295211 | −0.59229149 | 0.243611509 | 0.66162907 | 0 |
| 87 | hsa-miR-326 | hsa-miR-326 | −2.372605166 | −1.02946833 | 0.433897869 | 0.47503232 | 0 |
| 31 | hsa-miR-133b | hsa-miR-133b | −2.326329928 | −1.6790701 | 0.721767827 | 0.22502103 | 0 |
| 10 | hsa-miR-524* | hsa-miR-524* | −2.281702632 | −0.89378321 | 0.391717659 | 0.54933809 | 0 |
| 65 | hsa-miR-215 | hsa-miR-215 | −2.25191431 | −1.04481209 | 0.463966184 | 0.49169685 | 0 |
| 48 | hsa-miR-194 | hsa-miR-194 | −1.823265304 | −0.9350104 | 0.51282191 | 0.45462983 | 8.0886642 |
| 15 | hsa-miR-1 | hsa-miR-1 | −1.670332657 | −1.32505022 | 0.793285224 | 0.28201272 | 8.0886642 |
| 126 | hsa-let-7e | hsa-let-7e | −1.596893279 | −0.63719403 | 0.399021049 | 0.67031898 | 13.467626 |
| 94 | hsa-miR-381 | hsa-miR-381 | −1.593699657 | −0.40970867 | 0.257080226 | 0.75895339 | 13.467626 |
| 132 | hsa-miR-483 | hsa-miR-483 | −1.543748501 | −0.60908253 | 0.394547773 | 0.6729507 | 13.467626 |
| 21 | hsa-miR-10a | hsa-miR-10a | −1.528933362 | −0.52719213 | 0.344810404 | 0.703028 | 13.467626 |
| 90 | hsa-miR-365 | hsa-miR-365 | −1.493215754 | −0.49915012 | 0.334278632 | 0.69638992 | 13.467626 |
| 103 | hsa-miR-492 | hsa-miR-492 | −1.46308005 | −0.38342335 | 0.262065874 | 0.76032138 | 13.467626 |
| 120 | hsa-miR-99b | hsa-miR-99b | −1.454017884 | −0.48856399 | 0.336009614 | 0.73008171 | 13.467626 |
| 26 | hsa-miR-125b | hsa-miR-125b | −1.442119416 | −0.52412424 | 0.363440246 | 0.7200251 | 13.467626 |
| 16 | hsa-miR-100 | hsa-miR-100 | −1.375492355 | −0.54318835 | 0.394904663 | 0.71003751 | 14.388489 |
| 112 | hsa-miR-515-5p | hsa-miR-515-5p | −1.363339572 | −0.73965175 | 0.542529366 | 0.55193365 | 14.388489 |
| 89 | hsa-miR-335 | hsa-miR-335 | −1.29589199 | −0.64808235 | 0.500105224 | 0.68315652 | 14.388489 |
| 114 | hsa-miR-518b | hsa-miR-518b | −1.228635772 | −0.58333506 | 0.474782739 | 0.71938929 | 15.24114 |
| 12 | hsa-let-7c | hsa-let-7c | −1.175642329 | −0.5588639 | 0.475368987 | 0.68801083 | 22.31478 |

TABLE 10

| Gene | |
|---|---|
| Symbol | Description |
| CEBPB | CCAAT/enhancer-binding protein beta (C/EBP beta) |
| | (Nuclear factor NF- IL6) (Transcription factor 5). |
| BCL11A | B-cell lymphoma/leukemia 11A (B-cell CLL/lymphoma 11A) |
| | (COUP-TF- interacting protein 1) (Ecotropic viral |
| | integration site 9 protein) |
| ETS1 | C-ets-1 protein (p54) |
| ITK | Tyrosine-protein kinase ITK/TSK (EG 2.7.10.2) |
| | (T-cell-specific kinase) (Tyrosine-protein |
| | kinase Lyk) (Kinase EMT). |
| SP3 | Transcription factor Sp3 (SPR-2) |
| CSF1R | Macrophage colony-stimulating factor 1 receptor precursor |
| | (EC 2.7.10.1) (CSF-1-R) (Fms proto-oncogene) (c-fms) |
| | (CD115 antigen) |
| PCDH9 | Protocadherin-9 precursor |
| SOCS6 | Suppressor of cytokine signaling 6 (SOCS-6) |
| | (Suppressor of cytokine signaling 4) (SOCS-4) |
| RAPGEF4 | Rap guanine nucleotide exchange factor 4 (cAMP-regulated |
| | guanine nucleotide exchange factor II) (cAMP-GEFII) |
| RAB6A | Ras-related protein Rab-6A (Rab-6). [Source: |
| | Uniprot/SWISSPROT; Acc: P20340] |
| SOCS1 | Suppressor of cytokine signaling 1 (SOCS-1) |
| | (JAK-binding protein) (JAB) (STAT-induced STAT |
| | inhibitor 1) (SSI-1) |
| GDF6 | Growth/differentiation factor 6 precursor (GDF-6) |
| | (Growth/differentiation factor 16) |
| FGF7 | Keratinocyte growth factor precursor (KGF) |
| | (Fibroblast growth factor 7) (FGF-7) (HBGF-7) |
| CTLA4 | Cytotoxic T-lymphocyte protein 4 precursor |
| | (Cytotoxic T-lymphocyte- associated antigen |
| | 4) (CTLA-4) (CD152 antigen) |
| NFAT5 | Nuclear factor of activated T-cells 5 (T-cell transcription |
| | factor NFAT5) (NF-AT5) |

1. A method of diagnosing an inflammatory skin disorder in an individual comprising;
determining the expression of one or more of the miRNAs selected from the group consisting of miR-203, miR-155, miR-21, miR-125b, miR-31, miR-146a, miR-30c, miR-142-3p, miR-17-5p, miR-205, miR-99b, miR-193a, miR-133a, miR-326, let-7a, let-7b, let-7c, let-7d, let-7e, let-7f, let-7g, let-71, miR-1, miR-200a, miR-9, miR-10a, miR-10b, miR-15a, miR-15b, miR-16, miR-20a, miR-22, miR-23a, miR-23b, miR-24, miR-26a, miR-26b, miR-27a, miR-27b, miR-29a, miR-29c, miR-30a-5p, miR-30a-3p, miR-30b, miR-30d, miR-30e-5p, miR-30e-3p, miR-92, miR-93, miR-95, miR-99a, miR-100, miR-101, miR-106a, miR-106b, miR-107, miR-122a, miR-125a, miR-126, miR-127, miR-130a, miR-130b, miR-132, miR-133b, miR-135b, miR-140, miR-141, miR-142-5p, miR-143, miR-145, miR-146b, miR-148a, miR-148b, miR-149, miR-152, miR-181d, miR-182, miR-186, miR-187, miR-191, miR-193b, miR-194, miR-195, miR-196a, miR-196b, miR-197, miR-199a, miR-199b, miR-200b, miR-200c, miR-204, miR-214, miR-218, miR-222, miR-223, miR-224, miR-301, miR-320, miR-324-5p, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524* and miR-526b, miR-532, miR-615 and miR-660 in a sample of skin cells obtained from the individual,
wherein a change in expression of the one or more miRNAs in the sample relative to controls is indicative that the individual has an inflammatory skin disorder.

[symbol]

2. A method according to claim 1 comprising
determining the expression of one or more of the miRNAs selected from the group consisting of miR-155, miR-21, miR-31, miR-20a, miR-146a, miR-20a, miR-17-5p, miR-193a, miR-146b, miR-146a, miR-222, miR-142-3p, let-71, miR-106a, miR-200a, miR-30e-5p, miR-203, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, and miR-27a in the sample,
wherein an increase in the expression of one or more of these miRNAs in the sample relative to controls is indicative that the individual has an inflammatory skin disorder

3. A method according to claim 2 comprising
determining the expression of miR-155 and/or miR-21.

4. A method according to any one of claims 1 to 3 comprising
determining the expression of one or more of the miRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c in the sample,
wherein a decrease in the expression of one or more of these miRNAs in the sample relative to controls is indicative that the individual has an inflammatory skin disorder.

5. A method according to claim 4 comprising
determining the expression of miR-125b.

6. A method according to any one of the preceding claims wherein the inflammatory skin disorder is psoriasis or atopic eczema.

7. A method according to claim 1 wherein inflammatory skin disorder is psoriasis.

8. A method according to claim 7 comprising
determining the expression of one or more miRNAs selected from the group consisting of miR-203, miR-20a, miR-146a, miR-146b, miR-31, miR-146a, miR-20a, miR-200a, miR-17-5p, miR-30e-5p, miR-141, miR-142-3p, miR-21, miR-106a, miR-487b, miR-15a, let-71, miR-222, miR-422b, miR-130a, miR-193a, miR-106b, miR-199b, miR-199a*, miR-27b, miR-135b, miR-205, miR-155, miR-223, miR-93, miR-132, miR-425, miR-362, miR-324-5p, miR-224, miR-432 and miR-301 in the sample,
wherein an increase in expression of the one or more miRNAs in the sample relative to controls is indicative that the individual has psoriasis.

9. A method according to claim 8 comprising
determining the expression of miR-203 in the sample.

10. A method according to any one of claims 7 to 9 comprising;
determining the expression of one or more miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194, miR-526b, miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in the sample,
wherein a decrease in the expression of the one or more miRNAs in the sample relative to controls is indicative that the individual has psoriasis.

11. A method according to claim 1 wherein inflammatory skin disorder is atopic eczema.

12. A method according to claim 11 comprising
determining the expression of one or more miRNAs selected from the group consisting of miR-203, miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-223, miR-199a*, miR-200c, miR-155, miR-16, miR-361, miR-205, miR-143, miR-422b, miR-200b, miR-15b, miR-135b, miR-187, miR-142-5p, miR-501, miR-487b, miR-362, miR-301, miR-130b, and miR-146b,
wherein an increase in expression of the one or more miRNAs in the sample relative to controls in indicative that the individual has atopic eczema.

13. A method according to claim 11 or claim 12 comprising
determining the expression of one or more miRNAs selected from the group consisting of miR-122a, miR-133a-133b, miR-326, miR-215, miR-483, miR-519d, miR-335, miR-133b, miR-515-5p, miR-194, miR-524*, miR-197 miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451,
wherein a decrease in expression relative to controls in indicative that the individual has atopic eczema.

14. A method of treatment of an inflammatory skin disorder in an individual comprising;
increasing or reducing the expression or activity of one or more miRNAs selected from the group consisting of miR-203, miR-155, miR-21, miR-125b, miR-31, miR-146a, miR-30c, miR-142-3p, miR-17-5p, miR-205, miR-99b, miR-193a, miR-133a, miR-326, let-7a, let-7b, let-7c, let-7d, let-7e, let-7f, let-7g, let-71, miR-1, miR-9, miR-10a, miR-10b, miR-15a, miR-15b, miR-16, miR-20a, miR-22, miR-23a, miR-23b, miR-24, miR-26a, miR-26b, miR-27a, miR-27b, miR-29a, miR-29c, miR-30a-5p, miR-30a-3p, miR-30b, miR-30d, miR-30e-5p, miR-30e-3p, miR-92, miR-93, miR-95, miR-99a, miR-100, miR-101, miR-106a, miR-106b, miR-107, miR-122a, miR-125a, miR-126, miR-127, miR-130a, miR-130b, miR-132, miR-133b, miR-135b, miR-140, miR-141, miR-142-5p, miR-143, miR-145, miR-146b, miR-148a, miR-148b, miR-149, miR-152, miR-181d, miR-182, miR-186, miR-187, miR-191, miR-193b, miR-194, miR-195, miR-196a, miR-196b, miR-197, miR-199a, miR-199b, miR-200a, miR-200b, miR-200c, miR-204, miR-214, miR-218, miR-222, miR-223, miR-224, miR-301, miR-320, miR-324-5p, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524*, miR-526b, miR-532, miR-615 and miR-660 in cells of the individual.

15. A method according to claim 14 comprising;
reducing the expression or activity of one or more target miRNAs selected from the group consisting of miR-203, miR-20a, miR-146a, miR-20a, miR-31, miR-21, miR-17-5p, miR-193a, miR-146b, miR-146a, miR-222, miR-142-3p, let-71, miR-106a, miR-200a, miR-30e-5p, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155 in the cells.

[symbol]

16. A method according to claim 15 comprising reducing the expression or activity of miR-21.

17. A method according to claim 15 or claim 16 wherein the inflammatory skin disorder is psoriasis or atopic eczema.

18. A method according to claim 14 wherein inflammatory skin disorder is psoriasis.

19. A method according to claim 18 comprising;
reducing the expression or activity of one or more target miRNAs selected from the group consisting of miR-203, miR-20a, miR-146a, miR-146b, miR-31, miR-146a, miR-20a, miR-200a, miR-17-5p, miR-30e-5p, miR-141, miR-142-3p, miR-21, miR-106a, miR-487b, miR-15a, let-71, miR-222, miR-422b, miR-130a, miR-193a, miR-106b, miR-199b, miR-199a*, miR-27b, miR-135b, miR-205, miR-155, miR-223, miR-93, miR-132, miR-425, miR-362, miR-324-5p, miR-224, miR-432 and miR-301 in the cells.

20. A method according to claim 19 comprising
reducing the expression or activity of miR-203 and/or miR-146a.

21. A method according to claim 14 wherein inflammatory skin disorder is atopic eczema.

22. A method according to claim 21 comprising;
reducing the expression or activity of one or more target miRNAs selected from the group consisting of miR-203, miR-146a, let-7i, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-223, miR-199a*, miR-200c, miR-155, miR-16, miR-361, miR-205, miR-143, miR-422b, miR-200b, miR-15b miR-135b, miR-187, miR-142-5p, miR-501, miR-487b, miR-362, miR-301, miR-130b, and miR-146b.

23. A method according to any one of claims 14 to 22 wherein the expression or activity of one or more target miRNAs is reduced by administering a therapeutically effective amount of an miRNA inhibitor to the individual.

24. A method according to claim 23 wherein the miRNA inhibitor is a sense or anti-sense oligonucleotide which inhibits the activity or production of the target miRNA or increase its rate of depletion

25. A method according to claim 14 comprising;
increasing the expression or activity of one or more target miRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c in the cells.

26. A method according to claim 25 comprising
increasing the expression or activity of miR-125b.

27. A method according to any one of claims 25 to 26 wherein the inflammatory skin disorder is psoriasis or atopic eczema.

28. A method according to claim 14 wherein the inflammatory skin disorder is psoriasis.

29. A method according to claim 28 comprising increasing the amount or activity of one or target miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194, miR-526b, miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in the cells.

30. A method according to claim 14 wherein the inflammatory skin disorder is atopic eczema.

31. A method according to claim 30 comprising increasing the amount or activity of one or more target miRNAs selected from the group consisting of miR-122a, miR-133a-133b, miR-326, miR-215, miR-483, miR-519d, miR-335, miR-133b, miR-515-5p, miR-194, miR-524*, miR-197, miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451 in the cells.

32. A method according to any one of claims 25 to 31 wherein the expression or activity of the target miRNA is increased by administering to an individual in need thereof a therapeutically effective amount of;
(i) the target miRNA or a precursor thereof,
(ii) a nucleic acid encoding the target miRNA or a precursor thereof,
(iii) an analogue, derivative or modified form of the target miRNA which retains activity.

33. Use of an inhibitor of an miRNA selected from the group consisting of miR-203, miR-20a, miR-146a, miR-20a, miR-31, miR-21, miR-17-5p, miR-193a, miR-146b, miR-146a, miR-222, miR-142-3p, let-7i, miR-106a, miR-200a, miR-30e-5p, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155 in the manufacture of a medicament for use a method of treatment of an inflammatory skin disorder.

34. Use according to claim 33 wherein the method is a method according to any one of claims 14 to 24.

35. Use according to claim 33 or claim 34 wherein the miRNA inhibitor is a sense or anti-sense oligonucleotide which inhibits the activity or production of the target miRNA or increase its rate of depletion.

36. Use of an miRNA selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c or a precursor thereof, a nucleic acid encoding the miRNA or precursor thereof, or an analogue, derivative or modified form of the miRNA in the manufacture of a medicament for use a method of treatment of an inflammatory skin disorder.

37. Use according to claim 36 wherein the method is a method according to claim 14 or any one of claims 25 to 32.

38. A method of screening for a compound useful in the treatment of an inflammatory skin disorder comprising;
contacting a cell with a test compound and;
determining the expression of one or more miRNAs selected from the group consisting of miR-203, miR-155, miR-21, miR-125b, miR-31, miR-146a, miR-30c, miR-142-3p, miR-17-5p, miR-205, miR-99b, miR-193a, miR-133a, miR-326, let-7a, let-7b, let-7c, let-7d, let-7e, let-7f, let-7g, let-71, miR-1, miR-9, miR-10a, miR-10b, miR-15a, miR-15b, miR-16, miR-20a, miR-22, miR-23a, miR-23b, miR-24, miR-26a, miR-26b, miR-27a, miR-27b, miR-29a, miR-29c, miR-30a-5p, miR-30a-3p, miR-30b, miR-30d, miR-30e-5p, miR-30e-3p, miR-92, miR-93, miR-95, miR-99a, miR-100, miR-101, miR-106a, miR-106b, miR-107, miR-122a, miR-125a, miR-126, miR-127, miR-130a, miR-130b, miR-132, miR-133b, miR-135b, miR-140, miR-141, miR-142-5p, miR-143, miR-145, miR-146b, miR-148a, miR-148b, miR-149, miR-152, miR-181d, miR-182, miR-186, miR-187, miR-191, miR-193b, miR-194, miR-195, miR-196a, miR-196b, miR-197, miR-199a, miR-199b, miR-200a, miR-200b, miR-200c, miR-204, miR-214, miR-218, miR-222, miR-223, miR-224, miR-301, miR-320, miR-324-5p, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524*, miR-526b, miR-532, miR-615, and miR-660 relative to controls,
wherein an increase or decrease in expression in the presence of the test compound is indicative that the compound is useful in the treatment of an inflammatory skin disorder.

39. A method according to claim 38 comprising
determining the expression of one or more miRNAs selected from the group consisting of miR-203, miR-20a, miR-146a, miR-20a, miR-31, miR-21, miR-17-5p, miR-193a, miR-146b, miR-146a, miR-222, miR-142-3p, let-71, miR-106a, miR-200a, miR-30e-5p, miR-27b, miR-141, miR-130a, miR-24, miR-199b, miR-199a, miR-106b, miR-199a*, miR-15a, miR-451, miR-29a, miR-422b, miR-205, miR-361, miR-487b, miR-107, miR-451, miR-16, miR-27a and miR-155 in the cell,
wherein a decrease in expression in the presence of the test compound relative to its absence is indicative that the compound is useful in the treatment of an inflammatory skin disorder.

40. A method according to claim 39 comprising determining the expression of miR-21 and/or miR-155,
wherein a decrease in expression in the presence of the test compound relative to its absence is indicative that the compound is useful in the treatment of an inflammatory skin disorder.

41. A method according to claim 38 comprising
determining the expression of one or more miRNAs selected from the group consisting of miR-122a, miR-133a-133b, miR-197, miR-326, miR-133b, miR-524*, miR-215, miR-194, miR-1, let-7e, miR-381, miR-483, miR-10a, miR-365, miR-492, miR-99b, miR-125b, miR-100, miR-515-5p, miR-335, miR-518b and let-7c in the cell,
wherein an increase in expression of the one or more miRNAs in the presence of the test compound relative to its absence is indicative that the compound is useful in the treatment of an inflammatory skin disorder.

[symbol]

42. A method according to claim 41 comprising
determining the expression of miR-125b,
wherein an increase in expression in the presence of the test compound relative to its absence is indicative that the compound is useful in the treatment of an inflammatory skin disorder.

[symbol]

43. A method according to any one of claims 38 to 42 wherein the inflammatory skin disorder is psoriasis or atopic eczema.

44. A method according to claim 38 wherein inflammatory skin disorder is psoriasis.

45. A method according to claim 44 comprising
determining the expression of one or more miRNAs selected from the group consisting of miR-203, miR-20a, miR-146a, miR-146b, miR-31, miR-146a, miR-20a, miR-200a, miR-17-5p, miR-30e-5p, miR-141, miR-142-3p, miR-21, miR-106a, miR-487b, miR-15a, let-71, miR-222, miR-422b, miR-130a, miR-193a, miR-106b, miR-199b, miR-199a*, miR-27b, miR-135b, miR-205, miR-155, miR-223, miR-93, miR-132, miR-425, miR-362, miR-324-5p, miR-224, miR-432 and miR-301 in the cell,
wherein a decrease in expression in the presence of the test compound relative to its absence is indicative that the compound is useful in the treatment of psoriasis.

[symbol]

[symbol]

46. A method according to claim 44 or claim 45 comprising
determining the expression of one or more miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-184, miR-526b, miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in the cell,
wherein an increase in expression in the presence of the test compound relative to its absence is indicative that the compound is useful in the treatment of treatment of psoriasis.

47. A method according to claim 38 wherein inflammatory skin disorder is atopic eczema.

48. A method according to claim 47 comprising;
determining the expression of one or more microRNAs selected from the group consisting of miR-203, miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-451, miR-141, miR-31, miR-223, miR-199a*, miR-200c, miR-155, miR-16, miR-361, miR-205, miR-143, miR-422b, miR-200b, miR-15b, miR-135b, miR-187, miR-142-5p, miR-501, miR-487b, miR-362, miR-301, miR-130b, and miR-146b in the cell,
wherein a decrease in expression in the presence of the test compound relative to its absence is indicative that the compound is useful in the treatment of atopic eczema.

49. A method according to claim 47 comprising
determining the expression of one or more miRNAs selected from the group consisting of miR-122a, miR-133a-133b, miR-326, miR-215, miR-483, miR-519d, miR-335, miR-133b, miR-515-5p, miR-194, miR-524*, miR-197, miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451 in the cell,
wherein an increase in expression in the presence of the test compound relative to its absence is indicative that the compound is useful in the treatment of treatment of atopic eczema.

50. A method according to any one of claims 38 to 49 comprising identifying said test compound as a compound useful in the treatment of an inflammatory skin disorder.

51. A method according to claim 50 further comprising determining the ability of said test compound to ameliorate the symptoms of an inflammatory skin disorder in a non-human animal model.

52. A method according to claim 50 or claim 51 comprising isolating and/or purifying said test compound.

53. A method according to any one of claims 50 to 53 comprising producing and/or synthesising said test compound.

54. A method according to any one of claims 50 to 53 comprising formulating said test compound in a pharmaceutical composition with a pharmaceutically acceptable excipient, vehicle or carrier.

55. A method for monitoring the treatment of individual with a DNA damaging therapy comprising:
(a) subjecting the individual to a regimen of therapy for an inflammatory skin disorder; and
(b) monitoring in one or more cells of the individual the expression of one or more of the miRNAs selected from the group consisting of miR-203, miR-155, miR-21, miR-125b, miR-31, miR-146a, miR-30c, miR-142-3p, miR-17-5p, miR-205, miR-99b, miR-193a, miR-133a, miR-326, let-7a, let-7b, let-7c, let-7d, let-7e, let-7f, let-7g, let-71, miR-1, miR-200a, miR-9, miR-10a, miR-10b, miR-15a, miR-15b, miR-16, miR-20a, miR-22, miR-23a, miR-23b, miR-24, miR-26a, miR-26b, miR-27a, miR-27b, miR-29a, miR-29c, miR-30a-5p, miR-30a-3p, miR-30b, miR-30d, miR-30e-5p, miR-30e-3p, miR-92, miR-93, miR-95, miR-99a, miR-100, miR-101, miR-106a, miR-106b, miR-107, miR-122a, miR-125a, miR-126, miR-127, miR-130a, miR-130b, miR-132, miR-133b, miR-135b, miR-140, miR-141, miR-142-5p, miR-143, miR-145, miR-146b, miR-148a, miR-148b, miR-149, miR-152, miR-181d, miR-182, miR-186, miR-187, miR-191, miR-193b, miR-194, miR-195, miR-196a, miR-196b, miR-197, miR-199a, miR-199b, miR-200b, miR-200c, miR-204, miR-214, miR-218, miR-222, miR-223, miR-224, miR-301, miR-320, miR-324-5p, miR-328, miR-331, miR-335, miR-361, miR-362, miR-365, miR-375, miR-381, miR-383, miR-411, miR-422b, miR-425, miR-432, miR-451, miR-452, miR-483, miR-486, miR-487b, miR-492, miR-497, miR-501, miR-515-5p, miR-516-5p, miR-518b, miR-519d, miR-524* and miR-526b, miR-532, miR-615 and miR-660 during said treatment
wherein a change in expression is indicative that the regimen is efficacious in the individual.

56. A method according to claim 54 wherein inflammatory skin disorder is psoriasis.

57. A method according to claim 55 comprising
monitoring in one or more cells of the individual the expression of one or more of the miRNAs selected from the group consisting of miR-203, miR-20a, miR-146a, miR-146b, miR-31, miR-146a, miR-20a, miR-200a, miR-17-5p, miR-30e-5p, miR-141, miR-142-3p, miR-21, miR-106a, miR-487b, miR-15a, let-71, miR-222, miR-422b, miR-130a, miR-193a, miR-106b, miR-199b, miR-199a*, miR-27b, miR-135b, miR-205, miR-155, miR-223, miR-93, miR-132, miR-425, miR-362, miR-324-5p, miR-224, miR-432 and miR-301 in the sample,
wherein an decrease in expression of the one or more miRNAs in the sample after initiation of the regimen is indicative that the regimen is efficacious in the individual.

58. A method according to claim 55 comprising;
monitoring in one or more cells of the individual the expression of one or more of the miRNAs selected from the group consisting of miR-125b, miR-99b, miR-122a, miR-197, miR-100, miR-381, miR-518b, miR-524*, let-7e, miR-30c, miR-365, miR-133b, miR-10a, miR-133a-133b, miR-22, miR-326, miR-215, miR-516-5p, let-7c, let-7d, miR-335, miR-492, miR-1, miR-519d, miR-10b, miR-483, miR-194, miR-526b, miR-30a-3p, miR-195, miR-30a-5p, miR-30e-5p, miR-99a, miR-193b, miR-149, miR-26a, miR-26b, miR-218, miR-30e-3p, miR-23b, let-7g, miR-411, miR-199b, miR-29c, miR-101, miR-375, miR-214, miR-375, miR-181d, miR-125a, miR-140, miR-30b, miR-152, miR-328, miR-497, miR-130a, miR-127, miR-148b, miR-186, miR-143, miR-145, miR-30d, miR-126, miR-199a, miR-196b, miR-486, miR-365, miR-29a, miR-320, miR-361, miR-95, miR-532, miR-331, miR-199a, miR-196a, miR-451, miR-193a, miR-126, miR-23a, miR-660, let-7b, miR-16, miR-182, miR-27a, miR-383, let-7a and miR-191 in the sample,
wherein an increase in the expression of the one or more miRNAs in the sample after initiation of the regimen is indicative that the regimen is efficacious in the individual.

59. A method according to claim 55 wherein inflammatory skin disorder is atopic eczema.

60. A method according to claim 58 comprising
monitoring in one or more cells of the individual the expression of one or more of the miRNAs selected from the group consisting of miR-203, miR-146a, let-71, miR-29a, miR-222, miR-24, miR-193a, miR-199a, miR-27a, miR-21, miR-20a, miR-17-5p, miR-106b, miR-142-3p, miR-30e-5p, miR-107, miR-200a, miR-146b, miR-27b, miR-199b, miR-106a, miR-101, miR-451, miR-130a, miR-141, miR-31, miR-223, miR-199a*, miR-200c, miR-155, miR-16, miR-361, miR-205, miR-143, miR-422b, miR-200b, miR-15b, miR-135b, miR-187, miR-142-5p, miR-501, miR-487b, miR-362, miR-301, miR-130b, and miR-146b,
wherein an decrease in expression of the one or more miRNAs in the sample after initiation of the regimen is indicative that the regimen is efficacious in the individual.

61. A method according to claim 58 or claim 59 comprising
monitoring in one or more cells of the individual the expression of one or more of the miRNAs selected from the group consisting of miR-122a, miR-133a-133b, miR-326, miR-215, miR-483, miR-519d, miR-335, miR-133b, miR-515-5p, miR-194, miR-524*, miR-197 miR-99a, miR-383, miR-125b, miR-193b, miR-10a, miR-30a, miR-100, miR-328, miR-375, let-7c, miR-149, let-7a, miR-130a, miR-196b, miR-197, miR-26b, miR-30e-3p, miR-214, let-7b, miR-26a, miR-101, miR-30c, miR-195, let-7f, miR-148a, miR-125a, miR-30e-5p, miR-29c, miR-199b, miR-365, let-7d, miR-204, miR-23a, miR-196a, miR-452, miR-143, miR-199a, miR-145, miR-23b, miR-486, miR-92, miR-615, miR-497, miR-99b, miR-9, miR-10b, miR-1 and miR-451,
wherein an increase in expression of the one or more miRNAs in the sample after initiation of the regimen is indicative that the regimen is efficacious in the individual.
